SEQUENCE LISTING (56 sequences)

SEQ ID NO: 1 - protein, 120 aa, Artificial sequence
  synthetic sequence
QEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSS

SEQ ID NO: 2 - protein, 121 aa, Artificial sequence
  synthetic sequence
EVQLLESGGGLVQPGGSLRLSCAASGFSFSSSYYMCWVRQAPGKGLEWIACIYTSSGSAYYANWAKGRFTISRDNSKNTLYLQMNSLRAEDTAVYYCVRNAVGSSYYLYLWGQGTLVTVSS

SEQ ID NO: 3 - protein, 120 aa, Artificial sequence
  synthetic sequence
EVQLLESGGGLVQPGGSLRLSCAASGFSFSSSYYMCWVRQAPGKGLEWIACIYTSSGSAYYANWAKGRFTISRTSSTTVTLQMNSLRAEDTAVYYCVRNAVGSSYYLYLWGQGTLVTVSS

SEQ ID NO: 4 - protein, 121 aa, Artificial sequence
  synthetic sequence
EVQLLESGGGLVQPGGSLRLSCAASGFSFSSSYYMCWVRQAPGKGLEWIACIYTSSGSAYYADSVKGRFTISRDNSKNTLYLQMNSLRAEDTAVYYCVRNAVGSSYYLYLWGQGTLVTVSS

SEQ ID NO: 5 - protein, 120 aa, Artificial sequence
  synthetic sequence
EVQLLESGGGLVQPGGSLRLSCAASGFSFSSSYYMCWVRQAPGKGLEWIACIYTSSGSAYYADSVKGRFTISRTSSTTVTLQMNSLRAEDTAVYYCVRNAVGSSYYLYLWGQGTLVTVSS

SEQ ID NO: 6 - protein, 121 aa, Artificial sequence
  synthetic sequence
QVTLKESGPVLVKPTETLTLTCTASGFSFSSSYYMCWVRQPPGKALEWIACIYTSSGSAYYANWAKGRFTISRDTSKSQVVLTMTNMDPVDTATYFCVRNAVGSSYYLYLWGQGTLVTVSS

SEQ ID NO: 7 - protein, 110 aa, Artificial sequence
  synthetic sequence
AQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVK

SEQ ID NO: 8 - protein, 110 aa, Artificial sequence
  synthetic sequence
DIQMTQSPSTLSASVGDRVTITCQASQSVAGNNYLSWYQQKPGKAPKLLIYSVSTLASGVPSRFSGSGSGTEFTLTISSLQPDDFATYYCQGYYNDGIWAFGQGTKVEIK

SEQ ID NO: 9 - protein, 110 aa, Artificial sequence
  synthetic sequence
AQVLTQSPSTLSASVGDRVTITCQASQSVAGNNYLSWYQQKPGKAPKLLIYSVSTLASGVPSRFSGSGSGTEFTLTISSLQPDDFATYYCQGYYNDGIWAFGQGTKVEIK

SEQ ID NO: 10 - protein, 110 aa, Artificial sequence
  synthetic sequence
DIQMTQSPSSLSASVGDRVTITCQASQSVAGNNYLSWYQQKPGKVPKLLIYSVSTLASGVPSRFSGSGSGTDFTLTISSLQPEDVATYYCQGYYNDGIWAFGQGTKVEIK

SEQ ID NO: 11 - protein, 110 aa, Artificial sequence
  synthetic sequence
AQVLTQSPSSLSASVGDRVTITCQASQSVAGNNYLSWYQQKPGKVPKLLIYSVSTLASGVPSRFSGSGSGTDFTLTISSLQPEDVATYYCQGYYNDGIWAFGQGTKVEIK

SEQ ID NO: 12 - protein, 110 aa, Artificial sequence
  synthetic sequence
EQVLTQSPATLSLSPGERATLSCQASQSVAGNNYLSWYQQKPGQAPRLLIYSVSTLASGIPARFSGSGSGTDFTLTISSLEPEDFAVYYCQGYYNDGIWAFGQGTKLEIK

SEQ ID NO: 13 - protein, 6 aa, Artificial sequence
  synthetic sequence
SSYYMC

SEQ ID NO: 14 - protein, 17 aa, Artificial sequence
  synthetic sequence
  VARIANT (13)..(13): The amino acid at position 13 is Asn or Asp
  VARIANT (14)..(14): The amino acid at position 14 is Trp or Ser
  VARIANT (15)..(15): The amino acid at position 15 is Ala or Val
CIYTSSGSAYYAXXXKG

SEQ ID NO: 15 - protein, 17 aa, Artificial sequence
  synthetic sequence
CIYTSSGSAYYANWAKG

SEQ ID NO: 16 - protein, 17 aa, Artificial sequence
  synthetic sequence
CIYTSSGSAYYADSVKG

SEQ ID NO: 17 - protein, 11 aa, Artificial sequence
  synthetic sequence
NAVGSSYYLYL

SEQ ID NO: 18 - protein, 13 aa, Artificial sequence
  synthetic sequence
QASQSVAGNNYLS

SEQ ID NO: 19 - protein, 7 aa, Artificial sequence
  synthetic sequence
SVSTLAS

SEQ ID NO: 20 - protein, 10 aa, Artificial sequence
  synthetic sequence
QGYYNDGIWA

SEQ ID NO: 21 - protein, 317 aa, Vaccinia virus
MKTISVVTLLCVLPAVVYSTCTVPTMNNAKLTSTETSFNNNQKVTFTCDQGYHSSDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 22 - protein, 317 aa, Vaccinia virus
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 23 - protein, 317 aa, Vaccinia virus
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 24 - protein, 249 aa, Artificial sequence
  synthetic sequence
EVQLLESGGGLVQPGGSLRLSCAASGFSFSSSYYMCWVRQAPGKGLEWIACIYTSSGSAYYADSVKGRFTISRDNSKNTLYLQMNSLRAEDTAVYYCVRNAVGSSYYLYLWGQGTLVTVSSGSTSGSGKPGSGEGSTKGAQVLTQSPSTLSASVGDRVTITCQASQSVAGNNYLSWYQQKPGKAPKLLIYSVSTLASGVPSRFSGSGSGTEFTLTISSLQPDDFATYYCQGYYNDGIWAFGQGTKVEIK

SEQ ID NO: 25 - protein, 249 aa, Artificial sequence
  synthetic sequence
EVQLLESGGGLVQPGGSLRLSCAASGFSFSSSYYMCWVRQAPGKGLEWIACIYTSSGSAYYANWAKGRFTISRDNSKNTLYLQMNSLRAEDTAVYYCVRNAVGSSYYLYLWGQGTLVTVSSGSTSGSGKPGSGEGSTKGAQVLTQSPSSLSASVGDRVTITCQASQSVAGNNYLSWYQQKPGKVPKLLIYSVSTLASGVPSRFSGSGSGTDFTLTISSLQPEDVATYYCQGYYNDGIWAFGQGTKVEIK

SEQ ID NO: 26 - protein, 15 aa, Artificial sequence
  synthetic sequence
GGGGSGGGGSGGGGS

SEQ ID NO: 27 - protein, 22 aa, Artificial sequence
  synthetic sequence
MLLLVTSLLLCELPHPAFLLIP

SEQ ID NO: 28 - protein, 18 aa, Artificial sequence
  synthetic sequence
GSTSGSGKPGSGEGSTKG

SEQ ID NO: 29 - protein, 45 aa, Artificial sequence
  synthetic sequence
TTTPAPRPPTPAPTIASQPLSLRPEACRPAAGGAVHTRGLDFACD

SEQ ID NO: 30 - protein, 48 aa, Artificial sequence
  synthetic sequence
AKPTTTPAPRPPTPAPTIASQPLSLRPEACRPAAGGAVHTRGLDFACD

SEQ ID NO: 31 - protein, 62 aa, Artificial sequence
  synthetic sequence
GGGGSGGGGSGGGGSGGTTTPAPRPPTPAPTIASQPLSLRPEACRPAAGGAVHTRGLDFACD

SEQ ID NO: 32 - protein, 39 aa, Artificial sequence
  synthetic sequence
IEVMYPPPYLDNEKSNGTIIHVKGKHLCPSPLFPGPSKP

SEQ ID NO: 33 - protein, 10 aa, Artificial sequence
  synthetic sequence
GGGSSGGGSG

SEQ ID NO: 34 - protein, 12 aa, Artificial sequence
  synthetic sequence
ESKYGPPCPSCP

SEQ ID NO: 35 - protein, 106 aa, Artificial sequence
  synthetic sequence
GQPREPQVYTLPPSQEEMTKNQVSLTCLVKGFYPSDIAVEWESNGQPENNYKTTPPVLDSDGSFFLYSRLTVDKSRWQEGNVFSCVMHEALHNHYTQKSLSLSLGK

SEQ ID NO: 36 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 24 aa, Homo sapiens
IYIWAPLAGTCGVLLLSLVITLYC

SEQ ID NO: 38 - protein, 23 aa, Homo sapiens
IYIWAPLAGTCGVLLLSLVITLY

SEQ ID NO: 39 - protein, 22 aa, Homo sapiens
IYIWAPLAGTCGVLLLSLVITL

SEQ ID NO: 40 - protein, 27 aa, Homo sapiens
FWVLVVVGGVLACYSLLVTVAFIIFWV

SEQ ID NO: 41 - protein, 28 aa, Homo sapiens
MFWVLVVVGGVLACYSLLVTVAFIIFWV

SEQ ID NO: 42 - protein, 21 aa, Homo sapiens
LCYLLDGILFIYGVILTALFL

SEQ ID NO: 43 - protein, 22 aa, Homo sapiens
MALIVLGGVAGLLLFIGLGIFF

SEQ ID NO: 44 - protein, 27 aa, Homo sapiens
IISFFLALTSTALLFLLFFLTLRFSVV

SEQ ID NO: 45 - protein, 42 aa, Homo sapiens
KRGRKKLLYIFKQPFMRPVQTTQEEDGCSCRFPEEEEGGCEL

SEQ ID NO: 46 - protein, 41 aa, Homo sapiens
RSKRSRLLHSDYMNMTPRRPGPTRKHYQPYAPPRDFAAYRS

SEQ ID NO: 47 - protein, 41 aa, Homo sapiens
RSKRSRGGHSDYMNMTPRRPGPTRKHYQPYAPPRDFAAYRS

SEQ ID NO: 48 - protein, 42 aa, Homo sapiens
ALYLLRRDQRLPPDAHKPPGGGSFRTPIQEEQADAHSTLAKI

SEQ ID NO: 49 - protein, 112 aa, Artificial sequence
  synthetic sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 23 aa, Artificial sequence
  synthetic sequence
GSGQCTNYALLKLAGDVESNPGP

SEQ ID NO: 51 - protein, 21 aa, Artificial sequence
  synthetic sequence
GSGEGRGSLLTCGDVEENPGP

SEQ ID NO: 52 - protein, 239 aa, Artificial sequence
  synthetic sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 53 - protein, 236 aa, Artificial sequence
  synthetic sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 54 - protein, 357 aa, Homo sapiens
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 55 - protein, 494 aa, Artificial sequence
  synthetic sequence
MLLLVTSLLLCELPHPAFLLIPEVQLLESGGGLVQPGGSLRLSCAASGFSFSSSYYMCWVRQAPGKGLEWIACIYTSSGSAYYADSVKGRFTISRDNSKNTLYLQMNSLRAEDTAVYYCVRNAVGSSYYLYLWGQGTLVTVSSGSTSGSGKPGSGEGSTKGAQVLTQSPSTLSASVGDRVTITCQASQSVAGNNYLSWYQQKPGKAPKLLIYSVSTLASGVPSRFSGSGSGTEFTLTISSLQPDDFATYYCQGYYNDGIWAFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 494 aa, Artificial sequence
  synthetic sequence
MLLLVTSLLLCELPHPAFLLIPEVQLLESGGGLVQPGGSLRLSCAASGFSFSSSYYMCWVRQAPGKGLEWIACIYTSSGSAYYANWAKGRFTISRDNSKNTLYLQMNSLRAEDTAVYYCVRNAVGSSYYLYLWGQGTLVTVSSGSTSGSGKPGSGEGSTKGAQVLTQSPSSLSASVGDRVTITCQASQSVAGNNYLSWYQQKPGKVPKLLIYSVSTLASGVPSRFSGSGSGTDFTLTISSLQPEDVATYYCQGYYNDGIWAFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0309070 A1
(43) Pub. Date: Sep. 19, 2024

(54) ANTI-VACCINIA VIRUS ANTIGEN ANTIBODIES AND RELATED COMPOSITIONS AND METHODS

(71) Applicants: ADMARE THERAPEUTICS SOCIETY, Vancouver (CA); PROVINCIAL HEALTH SERVICES AUTHORITY, Vancouver (CA); UVIC INDUSTRY PARTNERSHIPS INC., Victoria (CA)

(72) Inventors: Emma J. Cummins, Vancouver (CA); Jan Peter Bergqvist, Vancouver (CA); Brad Nelson, Victoria (CA); Kwame Twumasi-Boateng, Nepean (CA); Yin Yu Eunice Kwok, Vancouver (CA); Francois Benard, Vancouver (CA); Julie Marie Rousseau, Vancouver (CA); Kuo-Shyan Lin, Richmond (CA); Julian Smazynski, Victoria (CA); Sander Martinus Johannes van Duijnhoven, Uden (NL)

(21) Appl. No.: 18/282,507

(22) PCT Filed: Mar. 16, 2022
(86) PCT No.: PCT/CA2022/050400

§ 371 Date: Sep. 15, 2023

Related U.S. Application Data
(60) Provisional application No. 63/195,536, filed on Jun. 1, 2021.
(60) Provisional application No. 63/162,199, filed on Mar. 17, 2021.

(51) Int. Cl. C07K 16/08; A61K 39/00; A61P 35/00; C07K 14/705; C07K 14/725; C12N 5/0783

(52) U.S. Cl. CPC C07K 16/081; A61K 39/4611; A61K 39/4631; A61K 39/464838; A61P 35/00; C07K 14/7051; C07K 14/70517; C07K 14/70578; C12N 5/0636; A61K 2239/15; A61K 2239/21; A61K 2239/22; C07K 2317/24; C07K 2317/565; C07K 2317/622; C07K 2319/03; C07K 2319/033; C12N 2510/00

(57) ABSTRACT

Provided are antibodies that specifically bind to Vaccinia Virus B5 antigen (VV B5). In certain embodiments, the anti-VV B5 antibodies are humanized antibodies. Fusion proteins and conjugates comprising such antibodies are also provided. Pharmaceutical compositions comprising the antibodies, fusion proteins and conjugates of the present disclosure are also provided, as are methods of using such compositions, e.g., for therapy, in vivo imaging and/or the like. In certain aspects, provided are methods that comprise administering an antibody, fusion protein or conjugate of the present disclosure to an individual, wherein the individual comprises cells infected with VV, and wherein the antibody, fusion protein or conjugate is targeted to the infected cells by VV B5 antigens expressed on the surface of the infected cells.

### CROSS-REFERENCE TO RELATED APPLICATIONS

This application claims the benefit of U.S. Provisional Patent Application No. 63/195,536, filed Jun. 1, 2021, and U.S. Provisional Patent Application No. 63/162,199, filed Mar. 17, 2021, which applications are incorporated herein by reference in their entireties.

### INTRODUCTION

Immunotherapy has emerged as an effective therapeutic option against multiple malignancies. Oncolytic viruses (OVs), which can be engineered to replicate selectively in and lyse tumor tissues while sparing the normal non-neoplastic host cells and simultaneously restoring antitumor immunity, constitute a next-generation immunotherapeutic approach for the treatment of tumors. The unique ability of OVs to target malignancies without dependence on specific antigen expression patterns makes them an attractive alternative to other immunotherapy approaches. In addition, OVs can promote the recruitment of tumor-infiltrating lymphocytes (TILs), reprogram the immunosuppressive tumor microenvironment (TME), and boost systemic antitumor immunity.

Genetic engineering has enabled the design of live replicating viruses to not only be highly tumor selective through cell entry and transcription targeting but also armed with reporter genes for noninvasive monitoring of the pharmacokinetics of virotherapy, and for enhancing cytotoxic activity or immunogenic cell death, or immune modulators. OVs in clinical development include measles virus, newcastle disease virus (NDV), rhabdoviruses, adenovirus, vaccinia virus (VV), herpes viruses, coxsackievirus, reovirus, and retrovirus.

Vaccinia virus (VV) is a large, enveloped, double-stranded DNA virus with a linear genome approximately 190 kb in length. Attenuation or tumor-specific targeting of these viruses has been accomplished using a variety of deletions and insertional mutations, with loss of thymidine kinase function being a common denominator among the clinical oncolytic vaccinia viruses. JX-594 is deleted for viral thymidine kinase, TG6002 is doubly deleted for thymidine kinase and viral ribonucleotide reductase, and GL-ONC1 has insertional mutations in its thymidine kinase (J2R), hemagglutinin HA (A56R), and F14.5L genes. The TK loss of function limits the virus' ability to replicate in non-dividing cells, and the deletion of viral ribonucleotide reductase further limits this ability. Two clinical vaccinia vectors designed to enhance oncolytic efficacy include transgenes designed to improve tumor cell killing: JX-594, like T-VEC, includes GM-CSF, and TG6002 incorporates a nucleoside analog converting enzyme FCU1, which converts 5-fluorocytosine (5-FC) to 5-FU in infected cells.

Despite the recent advances in OV-based therapies, there remains a need for new and improved OV-based methods for the treatment, alleviation, and/or prevention of cancer and for methods of improving survival in subjects with cancer.

### SUMMARY

Provided are antibodies that specifically bind to Vaccinia Virus B5 antigen (VV B5). In certain embodiments, the anti-VV B5 antibodies are humanized antibodies. Fusion proteins and conjugates comprising such antibodies are also provided. Pharmaceutical compositions comprising the antibodies, fusion proteins and conjugates of the present disclosure are also provided, as are methods of using such compositions, e.g., for therapy, in vivo imaging and/or the like. In certain aspects, provided are methods that comprise administering an antibody, fusion protein or conjugate of the present disclosure to an individual, wherein the individual comprises cells infected with VV, and wherein the antibody, fusion protein or conjugate is targeted to the infected cells by VV B5 antigens expressed on the surface of the infected cells.

### BRIEF DESCRIPTION OF THE FIGURES

FIG. 1 shows humanized antibody binding to VV B5 protein. Antibodies were assessed for binding to VV B5 monomer protein by ELISA. Humanized anti-B5 hIgG1 antibodies were titrated and showed similar pM EC50 compared to A048 parental positive control. No binding was observed with hIgG1 isotype control.

FIG. 2 shows humanized antibody binding to VV-infected cells. Antibodies were assessed for binding to VV infected cells by flow cytometry. HT29 cells were infected with VVddeGFP Western reserve virus (‘WR’) or VVCopenhagen (YFP) virus (‘Cop’), and SKOV3 cells were infected with VVddeGFP Western reserve virus (‘WR’). Humanized anti-B5 hIgG1 antibodies were titrated and showed similar nM EC50 compared to A048 parental anti-B5 hIgG1 (positive control). Negative control was human IgG1 isotype control.

FIG. 3 shows binding levels of humanized antibodies to different cell lines infected with Vaccinia virus over time. HT29 (A), SKOV3 (B), and OVCAR3 (C) cells were infected with Vaccinia virus strains VVddeGFP Western Reserve (‘WR’) or VVCopenhagen (YFP) virus (‘Cop’) at an MOI of 1 or 0.1, or mock-infected. Cells were harvested at the indicated timepoints to determine A048-H1L4 and A048-H3L2 binding levels in infected cells. Primary antibodies were conjugated to AlexaFluor-647. Data was acquired by a BD LSRFortessa X-20 and analyzed by FlowJo 10.8.1 software. Data represents biological triplicates. MOI, multiplicity of infection.

FIG. 4 shows a schematic of a design of humanized B5-CAR-050 or B5-CAR-051 chimeric antigen receptor (CAR) construct (FIG. 4A). Flow cytometry data showing humanized scFv CAR detection following lentiviral transduction and expansion of primary human T cells (FIG. 4B). Specific activation of human T cells expressing B5-CAR-050 or B5-CAR-051 when co-cultured with target expressing HT-29-B5, SKOV3-B5, and HEK293T-B5 lines (FIG. 4C). Primary human T cells expressing B5-CAR-050 or B5-CAR-051 showed upregulation of CD137 following co-culture with target B5 expressing tumor lines. Minimal cross-reactivity observed with WT target cell lines.

FIG. 5 shows data demonstrating that human T cells expressing B5-CAR-050 or B5-CAR-051 showed high percent specific cytotoxicity (FIG. 5A). Primary human T cells expressing B5-CAR-050 or B5-CAR-051 exhibited specific cell lysis at 24 h as determined by percent decrease of relative luminescence unit (RLU). Data demonstrating that primary human T cells expressing B5-CAR-050 or B5-CAR-051 showed morphological signs of direct tumor killing at 24 h following co-culture with target B5 expressing tumor lines (FIG. 5B).

FIG. 6 shows data demonstrating that human T cells expressing B5-CAR-050 or B5-CAR-051 showed high percent specific cytotoxicity against vaccinia virus infected target cell lines. Primary human T cells expressing B5-CAR-050 or B5-CAR-051 exhibited specific cell lysis at 24 h as determined by percent decrease of relative luminescence unit (RLU) against vaccinia virus infected target cells (FIG. 6A). In FIG. 6A, B5-CAR-050, B5-CAR-051 and parental B5-CAR-043 are identified as VV50_B5, VV51_B5 and VV43_B5_tEGFR, respectively. Data demonstrating that primary human T cells expressing B5-CAR-050 or B5-CAR-051 showed morphological signs of direct tumor killing at 24 h following co-culture with vaccinia virus infected tumor lines (FIG. 6B-6D).

FIG. 7 shows expected data demonstrating that human T cells expressing humanized B5-CAR result in diminished tumor growth when administered in combination with vaccinia virus used to treat a xenograft tumor model (FIG. 7A). Expected data demonstrating improved overall survival of the combined humanized B5-CAR and vaccinia virus therapy (FIG. 7B).

### DETAILED DESCRIPTION

Before the antibodies, compositions and methods of the present disclosure are described in greater detail, it is to be understood that the antibodies, compositions and methods are not limited to particular embodiments described, as such may, of course, vary. It is also to be understood that the terminology used herein is for the purpose of describing particular embodiments only, and is not intended to be limiting, since the scope of the antibodies, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the antibodies, compositions and methods. The upper and lower limits of these smaller ranges may independently be included in the smaller ranges and are also encompassed within the antibodies, compositions and methods, subject to any specifically excluded limit in the stated range. Where the stated range includes one or both of the limits, ranges excluding either or both of those included limits are also included in the antibodies, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the antibodies, compositions and methods belong. Although any antibodies, compositions and methods similar or equivalent to those described herein can also be used in the practice or testing of the antibodies, compositions and methods, representative illustrative antibodies, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present antibodies, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the antibodies, compositions and methods, which are, for clarity, described in the context of separate embodiments, may also be provided in combination in a single embodiment. Conversely, various features of the antibodies, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present antibodies, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Anti-VV B5 Antibodies

Aspects of the present disclosure include antibodies that specifically bind vaccinia virus (VV) B5 antigen (VV B5). In certain embodiments, the antibodies are humanized antibodies that specifically bind VV B5. Vaccinia viruses are members of the poxvirus family characterized by an approximately 192 kb double-stranded DNA genome that encodes numerous viral enzymes and factors that enable the virus to replicate independently from the host cell machinery. VV can stably accommodate up to 25 kb of cloned exogenous DNA. Structurally, it consists of a core region composed of viral DNA and various viral enzymes including RNA polymerase and polyA polymerase encased in a lipoprotein core membrane. The outer layer of the virus consists of double lipid membrane envelope. VV has inherent characteristics that make VV amenable for use in oncolytic viral therapy such as natural tropism for tumors, strong lytic ability, short life cycle with rapid cell-to-cell spread, efficient gene expression and a large cloning capacity. VV has a short life cycle of about 8 hours that takes place in the cytoplasm eliminating the risk of genome integration. Replication typically starts about 2 hours after infection, at which time host cell nucleic acid synthesis shuts down and cellular resources are directed toward viral replication. Cell lysis takes place between 12 and 48 hours releasing packaged viral particles. VV does not depend on host mechanisms for mRNA transcription making it less susceptible to biological changes of the host cell. Unlike other oncolytic viruses (OVs), VV does not require a specific surface receptor for cell entry, allowing it to infect a wide range of cells.

VV B5 protein is a 42 kDa type I transmembrane glycoprotein with an extracellular domain composed of four short consensus repeats (SCRs) characteristic of complement control proteins. After the SCRs, B5 has a stalk region before the transmembrane domain and a short cytoplasmic tail (CT). Both the SCRs and CT are dispensable for targeting B5 to the extracellular enveloped virus (EEV) membrane, although the latter affects its transport to the cell surface and recycling via endosomes. B5 is needed for intracellular mature virus (IMV) wrapping to form intracellular enveloped virus (IEV).

The term “antibody” (also used interchangeably with “immunoglobulin”) encompasses polyclonal (e.g., rabbit polyclonal) and monoclonal antibody preparations where the antibody may be an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the compound, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein. In some embodiments, the antibody is selected from an IgG, Fv, single chain antibody, scFv, a Fab, a F(ab′)₂, and a F(ab′). The antibodies may be further conjugated to other moieties, such as members of specific binding pairs, e.g., biotin (member of biotin-avidin specific binding pair), and the like.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, M D (1987); and Lefranc et al. IMGT, the international ImMunoGeneTics information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The CDRs of the antibodies provided by the present disclosure are defined according to Kabat, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, an antibody of the present disclosure comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies encompass intact immunoglobulins as well as a number of well characterized fragments which may be genetically encoded or produced by digestion with various peptidases. Thus, for example, pepsin digests an antibody below the disulfide linkages in the hinge region to produce F(ab)′₂, a dimer of Fab which itself is a light chain joined to VH-CHI by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, also includes antibody fragments either produced by the modification of whole antibodies or synthesized de novo using recombinant DNA methodologies, including but are not limited to, Fab′₂, IgG, IgM, IgA, scFv, dAb, nanobodies, unibodies, and diabodies. In certain embodiments, an antibody of the present disclosure is selected from an IgG, Fv, single chain antibody (e.g., scFv), Fab, F(ab′)₂, and Fab′.

According to some embodiments, an antibody of the present disclosure is a monoclonal antibody. “Monoclonal antibody” refers to a composition comprising one or more antibodies obtained from a population of substantially homogeneous antibodies, i.e., a population the individual antibodies of which are identical except for any naturally occurring mutations that may be present in minor amounts. Monoclonal antibodies are highly specific, being directed against a single antigenic site and generally to a single epitope on an antigen. The modifier “monoclonal” indicates the character of the antibody as being obtained from a substantially homogeneous population of antibodies, and does not require that the antibody be produced by any particular method or be the only antibody in the composition.

As summarized above, according to some embodiments, the antibodies of the present disclosure are humanized antibodies. In certain embodiments, provided are antibodies which are humanized versions of the parental rabbit antibody designated A048 described in International Patent Application No. PCT/CA202/051230 (WO 2021/046653), the disclosure of which is incorporated herein by reference in its entirety for all purposes. For example, provided herein are antibodies that comprise a V_{H} which is humanized relative to the V_{H} of the A048 antibody, a V_{L}which is humanized relative to the V_{L} of the A048 antibody, or both.

As used herein, a “humanized” antibody is a recombinant polypeptide that is derived from a non-human (e.g., rabbit, rodent, or the like) antibody and has been modified to contain at least a portion of the framework and/or constant regions of a human antibody. Humanized antibodies also encompass chimeric antibodies and CDR-grafted antibodies in which various regions may be derived from different species. Chimeric antibodies may be antibodies that include a variable region from any source linked to a human constant region (e.g., a human Fc domain). Thus, in chimeric antibodies, the variable region can be non-human, and the constant region is human. CDR-grafted antibodies are antibodies that include the CDRs from a non-human “donor” antibody linked to the framework region from a human “recipient” antibody. For example, an antibody of the present disclosure in a form of an scFV may be linked to a human constant region (e.g., Fc domain) to be made into a human immunoglobulin.

In general, humanized antibodies produce a reduced immune response in a human host (exhibit reduced immunogenicity), as compared to a non-humanized version of the same antibody. Antibodies can be humanized using a variety of techniques including, for example, CDR-grafting, veneering or resurfacing, chain shuffling, and the like. In certain embodiments, framework substitutions are identified by modeling of the interactions of the CDR and framework residues to identify framework residues important for antigen binding and sequence comparison to identify unusual framework residues at particular positions.

The substitution of rabbit or mouse CDRs into a human variable domain framework can result in retention of their correct spatial orientation where, e.g., the human variable domain framework adopts the same or similar conformation to the rabbit or mouse variable framework from which the CDRs originated. This can be achieved by obtaining the human variable domains from human antibodies whose framework sequences exhibit a high degree of sequence identity with the rabbit or mouse variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies.

Having identified the complementarity determining regions of the rabbit or mouse donor immunoglobulin and appropriate human acceptor immunoglobulins, a next step is to determine which, if any, residues from these components should be substituted to optimize the properties of the resulting humanized antibody. In general, substitution of human amino acid residues with rabbit or mouse should be minimized, because introduction of rabbit or mouse residues increases the risk of the antibody eliciting a human-anti-rabbit-antibody (HARA) or human-anti-mouse-antibody (HAMA) response in humans. Art-recognized methods of determining immune response can be performed to monitor a HARA or HAMA response in a particular patient or during clinical trials. Patients administered humanized antibodies can be given an immunogenicity assessment at the beginning and throughout the administration of said therapy. The HARA or HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HARA response in a human subject.

Certain amino acids from the human variable region framework residues are selected for substitution based on their possible influence on CDR conformation and/or binding to antigen. The unnatural juxtaposition of rabbit or murine CDR regions with human variable framework region can result in unnatural conformational restraints, which, unless corrected by substitution of certain amino acid residues, lead to loss of binding affinity. The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

When framework residues, as defined by, e.g., Kabat, constitute structural loop residues as defined by, e.g., Chothia, the amino acids present in the rabbit or mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk J M B 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody. Approaches that may be employed to humanize any of the antibodies described herein include, but are not limited to, those described in Williams, D., Matthews, D. & Jones, T. Humanising Antibodies by CDR Grafting. Antibody Engineering 319-339 (2010) doi:10.1007/978-3-642-01144-3_21; Kuramochi, T., Igawa, T., Tsunoda, H. & Hattori, K. Humanization and simultaneous optimization of monoclonal antibody. Methods Mol. Biol. 1060, 123-37 (2014); Hwang, W. Y., Almagro, J. C., Buss, T. N., Tan, P. & Foote, J. Use of human germline genes in a CDR homology-based approach to antibody humanization. Methods 36, 35-42 (2005); Lo, B. K. Antibody humanization by CDR grafting. Methods Mol. Biol. 248, 135-59 (2004); and Lefranc, M.-P. P., Ehrenmann, F., Ginestoux, C., Giudicelli, V. & Duroux, P. Use of IMGT(®) databases and tools for antibody engineering and humanization. Methods Mol. Biol. 907, 3-37 (2012); the disclosures of which are incorporated herein by reference in their entireties for all purposes.

The antibodies of the present disclosure specifically bind to the VV B5 antigen. An antibody “specifically binds” or “preferentially binds” to a target if it binds with greater affinity, avidity, more readily, and/or with greater duration than it binds to other substances, e.g., in a sample. In certain embodiments, an antibody “specifically binds” an antigen if it binds to or associates with the antigen with an affinity or Ka (that is, an association rate constant of a particular binding interaction with units of 1/M) of, for example, greater than or equal to about 10⁴ M⁻¹. Alternatively, affinity may be defined as an equilibrium dissociation constant (KD) of a particular binding interaction with units of M (e.g., 10⁻⁵ M to 10⁻¹³ M, or less). In certain aspects, specific binding means the antibody binds to the antigen with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for the antigen can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

According to some embodiments, provided are antibodies (non-humanized or humanized) that compete for binding to VV B5 antigen with any of the antibodies (non-humanized or humanized) described elsewhere herein. Whether an antibody of the present disclosure “competes with” a second antibody for binding to the antigen may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labeled. If the labeled antibody and the unlabeled antibody bind to separate and discrete sites on the antigen, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the antigen by about 50% or more, about 60% or more, about 70% or more, about 80% or more, about 85% or more, about 90% or more, about 95% or more, or about 99% or more. Details of procedures for carrying out such competition assays are known and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

In certain embodiments, the antibodies of the present disclosure are humanized versions of the parental rabbit antibody designated A048 described in International Patent Application No. PCT/CA202/051230. The amino acid sequences of the V_{H} and V_{L} polypeptides of the parental A048 antibody, as well as non-limiting examples of humanized versions of such VH and VL polypeptides (designated A048-H1-H5 and A048-L1-L5) are provided in Table 1 below (with CDRs underlined).

TABLE 1
Amino Acid Sequences of Example Humanized Anti-VV B5 Antibodies

| | |
|---|---|
| Parental A048 V_{H} | QEQLEESGGGLVKPEGSLTLTCTASGFSFSSSYYMCWVRQAPGRG |
| (SEQ ID NO: 1) | LEWIACIYTSSGSAYYANWAKGRFTISRTSSTTVTLQMTRLTAADTAT |
| | YFCVRNAVGSSYYLYLWGPGTLVTVSS |
| A048-H1 V_{H} | EVQLLESGGGLVQPGGSLRLSCAASGFSFSSSYYMCWVRQAPGKG |
| (SEQ ID NO: 2) | LEWIACIYTSSGSAYYANWAKGRFTISRDNSKNTLYLQMNSLRAEDT |
| | AVYYCVRNAVGSSYYLYLWGQGTLVTVSS |
| A048-H2 V_{H} | EVQLLESGGGLVQPGGSLRLSCAASGFSFSSSYYMCWVRQAPGKG |
| (SEQ ID NO: 3) | LEWIACIYTSSGSAYYANWAKGRFTISRTSSTTVTLQMNSLRAEDTA |
| | VYYCVRNAVGSSYYLYLWGQGTLVTVSS |
| A048-H3 V_{H} | EVQLLESGGGLVQPGGSLRLSCAASGFSFSSSYYMCWVRQAPGKG |
| (SEQ ID NO: 4) | LEWIACIYTSSGSAYYADSVKGRFTISRDNSKNTLYLQMNSLRAEDT |
| | AVYYCVRNAVGSSYYLYLWGQGTLVTVSS |
| A048-H4 V_{H} | EVQLLESGGGLVQPGGSLRLSCAASGFSFSSSYYMCWVRQAPGKG |
| (SEQ ID NO: 5) | LEWIACIYTSSGSAYYADSVKGRFTISRTSSTTVTLQMNSLRAEDTAV |
| | YYCVRNAVGSSYYLYLWGQGTLVTVSS |
| A048-H5 V_{H} | QVTLKESGPVLVKPTETLTLTCTASGFSFSSSYYMCWVRQPPGKAL |
| (SEQ ID NO: 6) | EWIACIYTSSGSAYYANWAKGRFTISRDTSKSQVVLTMTNMDPVDT |
| | ATYFCVRNAVGSSYYLYLWGQGTLVTVSS |
| Parental A048 V_{L} | AQVLTQTPSPVSAAVGGTVTISCQASQSVAGNNYLSWYQQKPGQP |
| (SEQ ID NO: 7) | PNLLIYSVSTLASGVPSRFKGSGSGTQFTLTISDLECDDAATYYCQG |
| | YYNDGIWAFGGGTEVVVK |
| A048-L1 V_{L} | DIQMTQSPSTLSASVGDRVTITCQASQSVAGNNYLSWYQQKPGKAP |
| (SEQ ID NO: 8) | KLLIYSVSTLASGVPSRFSGSGSGTEFTLTISSLQPDDFATYYCQGYY |
| | NDGIWAFGQGTKVEIK |
| A048-L2 V_{L} | AQVLTQSPSTLSASVGDRVTITCQASQSVAGNNYLSWYQQKPGKAP |
| (SEQ ID NO: 9) | KLLIYSVSTLASGVPSRFSGSGSGTEFTLTISSLQPDDFATYYCQGYY |
| | NDGIWAFGQGTKVEIK |
| A048-L3 V_{L} | DIQMTQSPSSLSASVGDRVTITCQASQSVAGNNYLSWYQQKPGKVP |
| (SEQ ID NO: 10) | KLLIYSVSTLASGVPSRFSGSGSGTDFTLTISSLQPEDVATYYCQGYY |
| | NDGIWAFGQGTKVEIK |
| A048-L4 V_{L} | AQVLTQSPSSLSASVGDRVTITCQASQSVAGNNYLSWYQQKPGKV |
| (SEQ ID NO: 11) | PKLLIYSVSTLASGVPSRFSGSGSGTDFTLTISSLQPEDVATYYCQGY |
| | YNDGIWAFGQGTKVEIK |
| A048-L5 V_{L} | EQVLTQSPATLSLSPGERATLSCQASQSVAGNNYLSWYQQKPGQA |
| (SEQ ID NO: 12) | PRLLIYSVSTLASGIPARFSGSGSGTDFTLTISSLEPEDFAVYYCQGY |
| | YNDGIWAFGQGTKLEIK |
| A048 V_{H} CDR1 | SSYYMC |
| (SEQ ID NO: 13) | |
| V_{H} CDR2 | CIYTSSGSAYYA(N/D)(W/S)(A/V)KG |
| (SEQ ID NO: 14) | |
| A048 V_{H} CDR2 | CIYTSSGSAYYANWAKG |
| (SEQ ID NO: 15) | |
| V_{H} CDR2 | CIYTSSGSAYYADSVKG |
| (SEQ ID NO: 16) | |
| A048 V_{H} CDR3 | NAVGSSYYLYL |
| (SEQ ID NO: 17) | |
| A048 V_{L} CDR1 | QASQSVAGNNYLS |
| (SEQ ID NO: 18) | |
| A048 V_{L} CDR2 | SVSTLAS |
| (SEQ ID NO: 19) | |
| A048 V_{L} CDR3 | QGYYNDGIWA |
| (SEQ ID NO: 20) | |

According to some embodiments, an antibody of the present disclosure comprises any desired combination of variable heavy chain (V_{H}) polypeptide set forth in Table 1 and variable light chain (V_{L}) polypeptide set forth in Table 1. By way of example, an antibody of the present disclosure may comprise the A048-H1 V_{H} paired with any of A048-L1 V_{L}, A048-L2 V_{L}, A048-L3 V_{L}, A048-L4 V_{L}, or A048-L5 V_{L}. Such an antibody may be designated A048-H1L1, A048-H1L2, A048-H1L3, A048-H1 L4, or A048-H1 L5, respectively. Also by way of example, an antibody of the present disclosure may comprise the A048-H2 V_{H} paired with any of A048-L1 V_{L}, A048-L2 V_{L}, A048-L3 V_{L}, A048-L4 V_{L}, or A048-L5 V_{L}. Such an antibody may be designated A048-H2L1, A048-H2L2, A048-H2L3, A048-H2L4, or A048-H2L5, respectively. As will be appreciated, an antibody of the present disclosure may comprise any combination of A048-H1 V_{H} to A048-H5 V_{H} and A048-L1 V_{L} to A048-L5 V_{L}.

In certain embodiments, an antibody of the present disclosure specifically binds to Vaccinia Virus B5 antigen (VV B5), wherein the antibody comprises:
a variable heavy chain (V_{H}) polypeptide comprising 70% or greater sequence identity to the amino acid sequence set forth in SEQ ID NO: 1, wherein the V_{H} polypeptide comprises:
   one or more mutations selected from the group consisting of: Q1E, E2V, Q3T, E5L/K, G9P, G10V, K13Q, E15G/T, G16E, S17T, T19R, T21S, T23A, A41P, R44K, G45A, T74D, S75N/T, S76_T77insK, T77N/S, T78Q, V79L, T80Y/V, Q82T, T84N, R85S/N, L86M, T87R/D, A88P, A89E/V, T93V, F95Y, P112Q, and any combination thereof, wherein numbering is according to SEQ ID NO:1;
   a V_{H} CDR1 comprising the amino acid sequence SSYYMC (SEQ ID NO:13),
   a V_{H} CDR2 comprising the amino acid sequence CIYTSSGSAYYA(N/D)(W/S)(A/V)KG (SEQ ID NO:14), and
   a V_{H} CDR3 comprising the amino acid sequence NAVGSSYYLYL (SEQ ID NO:17).

As used herein, a “mutation” encompasses an amino acid substitution, insertion of one or more amino acids, deletion of one or more amino acids, etc. relative to the parental V_{H} and V_{L} polypeptides of the A048 antibody set forth in SEQ ID NO:1 and SEQ ID NO:7, respectively. According to some embodiments, the V_{H} polypeptide of such an antibody comprises one, any combination of, or each of the mutations E2V, E5L/K, E15G/T, R44K, R85S/N, T87R/D, A89E/V, and P112Q. For example, the V_{H} polypeptide of such an antibody may comprise each of the mutations E2V, E5L/K, E15G/T, R44K, R85S/N, T87R/D, A89E/V, P112Q. In certain embodiments, the E5L/K mutation is E5L, the E15G/T mutation is E15G, the R85S/N mutation is R85S, the T87R/D mutation is T87R, and/or the A89E/V mutation is A89E. According to some embodiments, the V_{H} polypeptide of such an antibody comprises the mutations E5L, E15G, R85S, T87R, and A89E. In certain embodiments, the V_{H} polypeptide comprises one, any combination of, or each of the mutations Q1E, K13Q, T19R, T21S, T23A, T84N, T93V, and F95Y. According to some embodiments, the V_{H} polypeptide comprises one, any combination of, or each of the mutations T74D, S75N/T, S76_T77insK, T77N/S, V79L, and T80Y/V. As used herein, “S76_T77insK” means that the V_{H} polypeptide comprises an insertion of a K (lysine residue) between S76 and T77 relative to the parental A048 V_{H}. In certain embodiments, the T77N/S mutation is T77N. According to some embodiments, the T80Y/V mutation is T80Y. According to some embodiments, the V_{H} polypeptide comprises a V_{H} CDR2 comprising the amino acid sequence CIYTSSGSAYYADSVKG (SEQ ID NO:16).

According to some embodiments, the V_{H} polypeptide of such an antibody comprises one, any combination of, or each of the mutations Q3T, E5L/K, G9P, G10V, E15G/T, G16E, S17T, A41P, G45A, T74D, S75N/T, S76_T77insK, T77N/S, T78Q, T80Y/V, Q82T, R85S/N, L86M, T87R/D, A88P, and A89E/V. For example, the V_{H} polypeptide of such an antibody may comprise each of the mutations Q3T, E5L/K, G9P, G10V, E15G/T, G16E, S17T, A41P, G45A, T74D, S75N/T, S76_T77insK, T77N/S, T78Q, T80Y/V, Q82T, R85S/N, L86M, T87R/D, A88P, and A89E/V. In certain embodiments, the E5L/K mutation is E5K, the E15G/T mutation is E15T, the S75N/T mutation is S75T, the T77N/S mutation is T77S, the T80Y/V mutation is T80V, the R85S/N mutation is R85N, the T87R/D mutation is T87D, and/or the A89E/V mutation is A89V. According to some embodiments, the V_{H} polypeptide of such an antibody comprises the mutations E5K, E15T, R85N, T87D, and A89V.

In certain embodiments, the V_{H} polypeptide of a humanized antibody of the present disclosure comprises one or a desired combination of the mutations set forth above and comprises 80% or greater, 85% or greater, 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in one of SEQ ID Nos: 2-6.

In certain embodiments, an antibody of the present disclosure specifically binds to VV B5, wherein the antibody comprises:
a variable light chain (V_{L}) polypeptide comprising 70% or greater sequence identity to the amino acid sequence set forth in SEQ ID NO: 7, wherein the V_{L} polypeptide comprises:
   one or more mutations selected from the group consisting of: A1D/E, Q2I, V3Q, L4M, T7S, S9A, P10T/S, V11L, A13L, A14S, V15P, G17D/E, T18R, V19A, I21L, S22T, Q44K, P45A/V, N47K/R, V60I, S62A, K65S, Q72E/D, D79S, E81Q, C82P, D83E, A85F/V, T87V, G103Q, E106K, V107L, V108E, V109I, and any combination thereof, wherein numbering is according to SEQ ID NO: 7;
   a V_{L} CDR1 comprising the amino acid sequence QASQSVAGNNYLS (SEQ ID NO:18),
   a V_{L} CDR2 comprising the amino acid sequence SVSTLAS (SEQ ID NO:19), and
   a V_{L} CDR3 comprising the amino acid sequence QGYYNDGIWA (SEQ ID NO:20).

According to some embodiments, the V_{L} polypeptide of such an antibody comprises one, any combination of, or each of the mutations T7S, P10T/S, V11L, A14S, G17D/E, T18R, P45A/V, N47K/R, K65S, Q72E/D, D79S, C82P, A85F/V, G103Q, E106K, V108E, and V109I. For example, the V_{L} polypeptide of such an antibody may comprise each of the mutations T7S, P10T/S, V11L, A14S, G17D/E, T18R, P45A/V, N47K/R, K65S, Q72E/D, D79S, C82P, A85F/V, G103Q, E106K, V108E, and V109I. According to some embodiments, a V_{L} polypeptide of such an antibody comprises one, any combination of, or each of the mutations G17D, S22T, Q44K, N47K, and E81Q. In certain embodiments, the P10T/S mutation is P10T, the P45A/V mutation is P45A, the Q72E/D mutation is Q72E, and/or the A85F/V mutation is A85F. The V_{L} polypeptide of such an antibody may comprise one, any combination of, or each of the mutations A1D, Q2I, V3Q, and L4M.

In certain embodiments, the V_{L} polypeptide of an antibody of the present disclosure comprises the mutation D83E. According to some embodiments, the P10T/S mutation is P10S, the P45A/V mutation is P45V, the Q72E/D mutation is Q72D, and/or the A85F/V mutation is A85V. In certain embodiments, the V_{L} polypeptide comprises one, any combination of, or each of the mutations A1D, Q2I, V3Q, and L4M.

According to some embodiments, the V_{L} polypeptide of an antibody of the present disclosure comprises one, any combination of, or each of the mutations A1E, S9A, P10T, A13L, V15P, G17E, V19A, I21L, P45A, N47R, V60I, S62A, Q72D, D83E, A85F, T87V, and V107L. For example, the V_{L} polypeptide may comprise each of the mutations A1E, S9A, P10T, A13L, V15P, G17E, V19A, I21L, P45A, N47R, V60I, S62A, Q72D, D83E, A85F, T87V, and V107L.

In certain embodiments, the V_{L} polypeptide of a humanized antibody of the present disclosure comprises one or a desired combination of the V_{L} mutations set forth above and comprises 80% or greater, 85% or greater, 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in one of SEQ ID Nos: 8-12.

Also provided by the present disclosure are antibodies (non-humanized or humanized antibodies) that specifically bind VV B5, wherein the antibodies comprise:

a variable heavy chain (V_{H}) polypeptide comprising:

a V_{H} CDR 1 comprising the amino acid sequence
(SEQ ID NO: 13)
SSYYMC,
a V_{H} CDR2 comprising the amino acid sequence
(SEQ ID NO: 16)
CIYTSSGSAYYADSVKG,
and
a V_{H} CDR3 comprising the amino acid sequence
(SEQ ID NO: 17)
NAVGSSYYLYL;
and
a variable light chain (VL) polypeptide comprising:
a V_{L} CDR1 comprising the amino acid sequence
(SEQ ID NO: 18)
QASQSVAGNNYLS,
a V_{L} CDR2 comprising the amino acid sequence
(SEQ ID NO: 19)
SVSTLAS,
and
a V_{L} CDR3 comprising the amino acid sequence
(SEQ ID NO: 20)
QGYYNDGIWA.

The antibodies of the present disclosure specifically bind VV B5 antigen. Any of the antibodies may bind a VV B5 antigen from one or more VV strains, non-limiting examples of which include the B5 antigen of the Wyeth, Western Reserve and/or Copenhagen strains. The amino acid sequences of the B5 antigens encoded by these strains are provided in Table 2 below.

TABLE 2
B5 antigen sequences for Wyeth, Western Reserve and Copenhagen VV

| | |
|---|---|
| Wyeth B5 | MKTISVVTLLCVLPAVVYSTCTVPTMNNAKLTSTETSFNNNQKVTFT |
| (SEQ ID NO: 21) | CDQGYHSSDPNAVCETDKWKYENPCKKMCTVSDYVSELYDKPLY |
| | EVNSTMTLSCNGETKYFRCEEKNGNTSWNDTVTCPNAECQPLQL |
| | EHGSCQPVKEKYSFGEYITINCDVGYEVIGASYISCTANSWNVIPSC |
| | QQKCDIPSLSNGLISGSTFSIGGVIHLSCKSGFILTGSPSSTCIDGKW |
| | NPILPTCVRSNEKFDPVDDGPDDETDLSKLSKDVVQYEQEIESLEA |
| | TYHIIIVALTIMGVIFLISVIVLVCSCDKNNDQYKFHKLLP |
| Western Reserve B5 | MKTISVVTLLCVLPAVVYSTCTVPTMNNAKLTSTETSFNDKQKVTFT |
| (Uniprot Q01227) | CDQGYHSSDPNAVCETDKWKYENPCKKMCTVSDYISELYNKPLYE |
| (SEQ ID NO: 22) | VNSTMTLSCNGETKYFRCEEKNGNTSWNDTVTCPNAECQPLQLE |
| | HGSCQPVKEKYSFGEYMTINCDVGYEVIGASYISCTANSWNVIPSC |
| | QQKCDMPSLSNGLISGSTFSIGGVIHLSCKSGFTLTGSPSSTCIDGK |
| | WNPVLPICVRTNEEFDPVDDGPDDETDLSKLSKDVVQYEQEIESLE |
| | ATYHIIIVALTIMGVIFLISVIVLVCSCDKNNDQYKFHKLLP |
| Copenhagen B5 | MKTISVVTLLCVLPAVVYSTCTVPTMNNAKLTSTETSFNNNQKVTFT |
| (Uniprot P21115) | CDQGYHSSDPNAVCETDKWKYENPCKKMCTVSDYISELYNKPLYE |
| (SEQ ID NO: 23) | VNSTMTLSCNGETKYFRCEEKNGNTSWNDTVTCPNAECQPLQLE |
| | HGSCQPVKEKYSFGEYMTINCDVGYEVIGASYISCTANSWNVIPSC |
| | QQKCDIPSLSNGLISGSTFSIGGVIHLSCKSGFILTGSPSSTCIDGKW |
| | NPVLPICVRTNEEFDPVDDGPDDETDLSKLSKDVVQYEQEIESLEA |
| | TYHIIIVALTIMGVIFLISVIVLVCSCDKNNDQYKFHKLLP |

### Bispecific Antibodies

Also provided are bispecific antibodies. In certain embodiments, a bispecific antibody of the present disclosure comprises a first antigen-binding domain comprising a V_{H} polypeptide-V_{L} polypeptide pair of any of the anti-VV B5 antibodies of the present disclosure, including any of such antibodies described hereinabove. The bispecific antibody may include a second antigen-binding domain that specifically binds the VV B5 antigen bound by the first antigen-binding domain. In certain embodiments, the bispecific antibody includes a second antigen-binding domain that specifically binds a VV antigen other than the VV B5 antigen bound by the first antigen-binding domain.

According to some embodiments, a bispecific antibody of the present disclosure includes a second antigen-binding domain that specifically binds an antigen other than a VV antigen. In certain embodiments, the antigen other than a VV antigen is an immune cell surface antigen. Non-limiting examples of immune cell surface antigens are immune effector cell surface antigens, e.g., a T cell surface antigen, a natural killer (NK) cell surface antigen, a macrophage cell surface antigen, and the like. Examples of T cell surface antigens that may be bound by the second antigen-binding domain include, but are not limited to, a T cell stimulatory molecule, e.g., CD3, CD28, etc.

Bispecific antibodies of the present disclosure include antibodies having a full-length antibody structure, and bispecific antibody fragments. “Full-length” as used herein refers to an antibody having two full-length antibody heavy chains and two full length antibody light chains. A full-length antibody heavy chain (HC) consists of well-known heavy chain variable and constant domains VH, CH1, CH2, and CH3. A full-length antibody light chain (LC) consists of well-known light chain variable and constant domains VL and CL. The full-length antibody may be lacking the C-terminal lysine in either one or both heavy chains. The term “Fab arm” refers to one heavy chain:light chain pair that specifically binds an antigen.

Full-length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in a cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., WO 2006/028936) may be used to generate full length bispecific antibodies. Briefly, selected amino acids forming the interface of the CHS domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US2010/0015133; US2009/0182127; US2010/028637 or US2011/0123532. In other strategies. heterodimerization may be promoted by the following substitutions (expressed as modified position in the first CH3 domain of the first heavy chain/modified position in the second CH3 domain of the second heavy chain): L351 Y_F405A_Y407V T394W, T366I_K392M_T394W/F405A_Y407V, T366L_K392M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

Also provided are single chain bispecific antibodies. In some embodiments, a single chain bispecific antibody of the present disclosure is a bispecific scFv. Details regarding bispecific scFvs may be found, e.g., in Zhou et al. (2017) J Cancer 8(18):3689-3696.

Approaches that may be employed to produce multispecific (e.g., bispecific) antibodies from the antibodies described herein include, but are not limited to, Ellerman, D. (2019). “Bispecific T-cell engagers: Towards understanding variables influencing the in vitro potency and tumor selectivity and their modulation to enhance their efficacy and safety.” Methods 154: 102-117; Brinkmann, U. and R. E. Kontermann (2017). “The making of bispecific antibodies.” mAbs 9(2): 182-212; and Suurs, F. V., et al. (2019). “A review of bispecific antibodies and antibody constructs in oncology and clinical challenges.” Pharmacol Ther 201: 103-119; the disclosures of which are incorporated herein by reference in their entireties for all purposes.

### Fusion Proteins

Aspects of the present disclosure further include fusion proteins. In certain embodiments, a fusion protein of the present disclosure comprises a V_{H} polypeptide, a V_{L} polypeptide, or both, of any of the anti-VV B5 antibodies of the present disclosure, fused to a heterologous sequence of amino acids. The heterologous sequence of amino acids may be fused to the C-terminus of the chain of the antibody or the N-terminus of the chain of the antibody. In certain embodiments, a fusion protein of the present disclosure includes a heterologous sequence at the C-terminus of the chain of the antibody and a heterologous sequence at the N-terminus of the chain of the antibody, wherein the heterologous sequences may be the same sequence or different sequences. “Heterologous” as used in the context of a nucleic acid or polypeptide generally means that the nucleic acid or polypeptide is from a different origin (e.g., molecule of different sequence, different species origin, and the like) than that with which the nucleic acid or polypeptide is associated or joined, such that the nucleic acid or polypeptide is one that is not found in nature. For example, in a fusion protein, a light chain polypeptide and a reporter polypeptide (e.g., GFP, red fluorescent protein (e.g., mCherry), luciferase, etc.) are said to be “heterologous” to one another. Similarly, a CDR from a rabbit antibody and a constant region from a human antibody are “heterologous” to one another.

The V_{H} polypeptide and/or V_{L} polypeptide may be fused to any heterologous sequence of interest. Heterologous sequences of interest include, but are not limited to, an albumin, a transferrin, XTEN, a homo-amino acid polymer, a proline-alanine-serine polymer, an elastin-like peptide, or any combination thereof. In certain aspects, the heterologous polypeptide increases the stability and/or serum half-life of the anti-VV B5 antibody upon its administration to an individual in need thereof, as compared to the same antibody which is not fused to the heterologous sequence.

In certain embodiments, a fusion protein of the present disclosure comprises a single chain antibody, e.g., a single chain antibody (e.g., scFv) comprising a V_{H} polypeptide-V_{L} polypeptide pair of any of the anti-VV B5 antibodies of the present disclosure, including any of such antibodies described hereinabove. The amino acid sequences of non-limiting examples of humanized scFvs according to embodiments of the present disclosure are provided in Table 3 below.

TABLE 3
Example Anti-VV B5 scFv Amino Acid Sequences

| | |
|---|---|
| scFv of B5-CAR-050 | EVQLLESGGGLVQPGGSLRLSCAASGFSFSSSYYMCWVRQAPGKGL |
| •V_{H} (A048 H3) | EWIACIYTSSGSAYYADSVKGRFTISRDNSKNTLYLQMNSLRAEDTAVY |
| •Linker | YCVRNAVGSSYYLYLWGQGTLVTVSSGSTSGSGKPGSGEGSTKGAQ |
| •V_{L} (A048 L2) | VLTQSPSTLSASVGDRVTITCQASQSVAGNNYLSWYQQKPGKAPKLLI |
| (SEQ ID NO: 24) | YSVSTLASGVPSRFSGSGSGTEFTLTISSLQPDDFATYYCQGYYNDGI |
| | WAFGQGTKVEIK |
| scFv of B5-CAR-051 | EVQLLESGGGLVQPGGSLRLSCAASGFSFSSSYYMCWVRQAPGKGL |
| •V_{H} (A048 H1) | EWIACIYTSSGSAYYANWAKGRFTISRDNSKNTLYLQMNSLRAEDTAV |
| •Linker | YYCVRNAVGSSYYLYLWGQGTLVTVSSGSTSGSGKPGSGEGSTKGA |
| •V_{L} (A048 L4) | QVLTQSPSSLSASVGDRVTITCQASQSVAGNNYLSWYQQKPGKVPKL |
| (SEQ ID NO: 25) | LIYSVSTLASGVPSRFSGSGSGTDFTLTISSLQPEDVATYYCQGYYNDG |
| | IWAFGQGTKVEIK |

Also provided are the anti-VV B5 scFvs set forth in Table 3, but where the orientation of the V_{H} and V_{L} is reversed—that is, where the V_{L} is N-terminal to the V_{H}.

According to some embodiments, when the fusion protein comprises a single chain antibody (e.g., any of the single chain antibodies of the present disclosure, including any of the scFvs described herein), the fusion protein is a chimeric antigen receptor (CAR) comprising the single chain antibody, a transmembrane domain, and an intracellular signaling domain.

A CAR of the present disclosure may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a serine-glycine linker, such as a serine-glycine linker that includes the amino acid sequence GGGGSGGGGSGGGGS (G₄S)₃ (SEQ ID NO:26). In certain embodiments, a linker separates one or more heavy or light chain variable domains, hinge domains, transmembrane domains, co-stimulatory domains, and/or primary signaling domains. In particular embodiments, the CAR includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, the antigen binding domain of the CAR is followed by one or more spacer domains that moves the antigen binding domain away from the effector cell surface (e.g., the surface of a T cell expressing the CAR) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In one embodiment, the spacer domain includes the CH2 and/or CH3 of IgG1, IgG4, or IgD. Illustrative spacer domains suitable for use in the CARs described herein include the hinge region derived from the extracellular regions of type 1 membrane proteins such as CD8α and CD4, which may be wild-type hinge regions from these molecules or variants thereof. In certain aspects, the hinge domain includes a CD8α hinge region. In some embodiments, the hinge is a PD-1 hinge or CD152 hinge.

The “transmembrane domain” (Tm domain) is the portion of the CAR that fuses the extracellular binding portion and intracellular signaling domain and anchors the CAR to the plasma membrane of the cell (e.g., immune effector cell). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, or PD-1.

In one embodiment, a CAR includes a Tm domain derived from CD8α. In certain aspects, a CAR includes a Tm domain derived from CD8α and a short oligo- or polypeptide linker, e.g., between 1, 2, 3, 4, 5, 6, 7, 8, 9, or 10 amino acids in length, that links the Tm domain and the intracellular signaling domain of the CAR. A glycine-serine linker may be employed as such a linker, for example.

The “intracellular signaling” domain of a CAR refers to the part of a CAR that participates in transducing the signal from CAR binding to a target molecule/antigen into the interior of the immune effector cell to elicit effector cell function, e.g., activation, cytokine production, proliferation and/or cytotoxic activity, including the release of cytotoxic factors to the CAR-bound target cell, or other cellular responses elicited with target molecule/antigen binding to the extracellular CAR domain. Accordingly, the term “intracellular signaling domain” refers to the portion of a protein which transduces the effector function signal and that directs the cell to perform a specialized function. To the extent that a truncated portion of an intracellular signaling domain is used, such truncated portion may be used in place of a full-length intracellular signaling domain as long as it transduces the effector function signal. The term intracellular signaling domain is meant to include any truncated portion of an intracellular signaling domain sufficient for transducing effector function signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a CAR of the present disclosure may include an intracellular signaling domain that includes one or more “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a CAR of the present disclosure include those derived from FcRγ, FcRβ, CD3γ, CD3δ, CD3ε, CD3ζ, CD22, CD79α, CD79β, and CD66δ. In certain embodiments, a CAR includes a CD3ζ primary signaling domain and one or more costimulatory signaling domains. The intracellular primary signaling and costimulatory signaling domains are operably linked to the carboxyl terminus of the transmembrane domain.

In some embodiments, the CAR includes one or more costimulatory signaling domains to enhance the efficacy and expansion of immune effector cells (e.g., T cells) expressing the CAR.

As used herein, the term “costimulatory signaling domain” or “costimulatory domain” refers to an intracellular signaling domain of a costimulatory molecule or an active fragment thereof. Example costimulatory molecules suitable for use in CARs contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the CAR includes one or more costimulatory signaling domains selected from the group consisting of 4-1BB (CD137), CD28, and CD134, and a CD3ζ primary signaling domain.

A CAR of the present disclosure may include any variety of suitable domains including but not limited to a leader sequence; hinge, spacer and/or linker domain(s); transmembrane domain(s); costimulatory domain(s); signaling domain(s) (e.g., CD3ζ domain(s)); ribosomal skip element(s); restriction enzyme sequence(s); reporter protein domains; and/or the like. Non-limiting examples of such domains that may be included in a CAR of the present disclosure include those provided in Table 4 below. As will be appreciated by one of ordinary skill in the art, the amino acid sequence of one or more of the domains indicated in Table 4 (e.g., linker, hinge, transmembrane, co-stimulatory, signaling, ribosomal skip element; restriction enzyme sequence; reporter protein etc.) may be modified as desired, e.g., for improved functionality, etc. of the CAR.

TABLE 4
Example CAR Domain Amino Acid Sequences
Leader Signal Peptide(s)

| | |
|---|---|
| GM-CSFR alpha (P15509) | MLLLVTSLLLCELPHPAFLLIP |
| (SEQ ID NO: 27) | |

Hinge/Spacer/Linker domain(s)

| | |
|---|---|
| (SEQ ID NO: 28) | GSTSGSGKPGSGEGSTKG |
| (SEQ ID NO: 26) | GGGGSGGGGSGGGGS |
| CD8a hinge | TTTPAPRPPTPAPTIASQPLSLRPEACRPAAGGAVHTRGLDFACD |
| (SEQ ID NO: 29) | |
| CD8a hinge 2 | AKPTTTPAPRPPTPAPTIASQPLSLRPEACRPAAGGAVHTRGLDFAC |
| (SEQ ID NO: 30) | D |
| extended CD8 hinge | GGGGSGGGGSGGGGSGGTTTPAPRPPTPAPTIASQPLSLRPEACRP |
| (SEQ ID NO: 31) | AAGGAVHTRGLDFACD |
| CD28 hinge | IEVMYPPPYLDNEKSNGTIIHVKGKHLCPSPLFPGPSKP |
| (SEQ ID NO: 32) | |
| GSG linker | GGGSSGGGSG |
| (SEQ ID NO: 33) | |
| IgG4 hinge | ESKYGPPCPSCP |
| (SEQ ID NO: 34) | |
| IgG4(CH3) | GQPREPQVYTLPPSQEEMTKNQVSLTCLVKGFYPSDIAVEWESNGQ |
| (SEQ ID NO: 35) | PENNYKTTPPVLDSDGSFFLYSRLTVDKSRWQEGNVFSCVMHEALH |
| | NHYTQKSLSLSLGK |
| IgG4 (P01861) | ESKYGPPCPSCPAPEFLGGPSVFLFPPKPKDTLMISRTPEVTCVVVD |
| (SEQ ID NO: 36) | VSQEDPEVQFNWYVDGVEVHNAKTKPREEQFNSTYRVVSVLTVLHQ |
| | DWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFF |
| | LYSRLTVDKSRWQEGNVFSCSVMHEALHNHYTQKSLSLSLGK |

Transmembrane domain(s)

| | |
|---|---|
| CD8tm (NM_001768) | IYIWAPLAGTCGVLLLSLVITLYC |
| (SEQ ID NO: 37) | |
| CD8tm2 (NM_001768) | IYIWAPLAGTCGVLLLSLVITLY |
| (SEQ ID NO: 38) | |
| CD8tm3 (NM_001768) | IYIWAPLAGTCGVLLLSLVITL |
| (SEQ ID NO: 39) | |
| CD28tm (NM_006139) | FWVLVVVGGVLACYSLLVTVAFIIFWV |
| (SEQ ID NO: 40) | |
| CD28tm2 (NM_006139) | MFWVLVVVGGVLACYSLLVTVAFIIFWV |
| (SEQ ID NO: 41) | |
| CD3z (J04132.1) | LCYLLDGILFIYGVILTALFL |
| (SEQ ID NO: 42) | |
| CD4tm (M35160) | MALIVLGGVAGLLLFIGLGIFF |
| (SEQ ID NO: 43) | |
| 4-1BB (NM_001561) | IISFFLALTSTALLFLLFFLTLRFSVV |
| (SEQ ID NO: 44) | |

Costimulatory domain(s)

| | |
|---|---|
| 4-1BB (NM_001561) | KRGRKKLLYIFKQPFMRPVQTTQEEDGCSCRFPEEEEGGCEL |
| (SEQ ID NO: 45) | |
| CD28 (NM_006139) | RSKRSRLLHSDYMNMTPRRPGPTRKHYQPYAPPRDFAAYRS |
| (SEQ ID NO: 46) | |
| CD28gg (NM_006139) | RSKRSRGGHSDYMNMTPRRPGPTRKHYQPYAPPRDFAAYRS |
| (SEQ ID NO: 47) | |
| OX40 (P43489) | ALYLLRRDQRLPPDAHKPPGGGSFRTPIQEEQADAHSTLAKI |
| (SEQ ID NO: 48) | |

CD3ζ domain(s)

| | |
|---|---|
| CD3ζ | RVKFSRSADAPAYQQGQNQLYNELNLGRREEYDVLDKRRGRDPEM |
| (SEQ ID NO: 49) | GGKPRRKNPQEGLYNELQKDKMAEAYSEIGMKGERRRGKGHDGLY |
| | QGLSTATKDTYDALHMQALPPR |

Ribosomal Skip Element(s)

| | |
|---|---|
| E2A | GSGQCTNYALLKLAGDVESNPGP |
| (SEQ ID NO: 50) | |
| T2A | GSGEGRGSLLTCGDVEENPGP |
| (SEQ ID NO: 51) | |

Restriction Enzyme Sequence(s)

| | |
|---|---|
| PacI | LIN |

Reporter Protein(s)

| | |
|---|---|
| eGFP | MVSKGEELFTGVVPILVELDGDVNGHKFSVSGEGEGDATYGKLTLKFI |
| (SEQ ID NO: 52) | CTTGKLPVPWPTLVTTLTYGVQCFSRYPDHMKQHDFFKSAMPEGYV |
| | QERTIFFKDDGNYKTRAEVKFEGDTLVNRIELKGIDFKEDGNILGHKLE |
| | YNYNSHNVYIMADKQKNGIKVNFKIRHNIEDGSVQLADHYQQNTPIGD |
| | GPVLLPDNHYLSTQSALSKDPNEKRDHMVLLEFVTAAGITLGMDELY |
| | K |
| mCherry | MVSKGEEDNMAIIKEFMRFKVHMEGSVNGHEFEIEGEGEGRPYEGT |
| (SEQ ID NO: 53) | QTAKLKVTKGGPLPFAWDILSPQFMYGSKAYVKHPADIPDYLKLSFPE |
| | GFKWERVMNFEDGGVVTVTQDSSLQDGEFIYKVKLRGTNFPSDGPV |
| | MQKKTMGWEASSERMYPEDGALKGEIKQRLKLKDGGHYDAEVKTTY |
| | KAKKPVQLPGAYNVNIKLDITSHNEDYTIVEQYERAEGRHSTGGMDE |
| | LYK |
| Truncated EGFR (partial | MLLLVTSLLLCELPHPAFLLIPRKVCNGIGIGEFKDSLSINATNIKHFKN |
| sequence from UniProtKB- | CTSISGDLHILPVAFRGDSFTHTPPLDPQELDILKTVKEITGFLLIQAWP |
| P00533) | ENRTDLHAFENLEIIRGRTKQHGQFSLAVVSLNITSLGLRSLKEISDGD |
| (SEQ ID NO: 54) | VIISGNKNLCYANTINWKKLFGTSGQKTKIISNRGENSCKATGQVCHA |
| | LCSPEGCWGPEPRDCVSCRNVSRGRECVDKCNLLEGEPREFVENS |
| | ECIQCHPECLPQAMNITCTGRGPDNCIQCAHYIDGPHCVKTCPAGVM |
| | GENNTLVWKYADAGHVCHLCHPNCTYGCTGPGLEGCPTNGPKIPSI |
| | ATGMVGALLLLLVVALGIGLFM |

In certain aspects, a CAR of the present disclosure comprises a single chain antibody (e.g., any of the scFvs of the present disclosure) that specifically binds VV B5 antigen; a transmembrane domain from a polypeptide selected from the group consisting of: 004, CD8α, 00154, and PD-i; one or more intracellular costimulatory signaling domains from a polypeptide selected from the group consisting of: 4-l1E1 (00137), 0028, and 00134; and an intracellular signaling domain from a polypeptide selected from the group consisting of: FcRγ, FcRβ, CD3γ, CD3δ, CD3ε, CD3ζ, CD22, CD79α, CD79β, and CD66δ. Such a CAR may further include a spacer domain between the antigen-binding portion and the transmembrane domain, e.g., a CD8 alpha hinge.

According to some embodiments, provided are CARs that comprise—from N-terminus to C-terminus—a variable heavy chain (V_{H}) polypeptide of an antibody described herein, a linker, a variable light chain (V_{L}) polypeptide of an antibody described herein, a CD8 hinge region (which in some embodiments is an extended CD8 hinge region), a CD8 transmembrane domain, a 4-1BB costimulatory domain, and a CD3ζ signaling domain. According to certain embodiments, provided are CARs that comprise—from N-terminus to C-terminus—a variable light chain (V_{L}) polypeptide of an antibody described herein, a linker, a variable heavy chain (V_{H}) of an antibody described herein, a CD8 hinge region (which in some embodiments is an extended CD8 hinge region), a CD8 transmembrane domain, a 4-1EE costimulatory domain, and a CD3ζ signaling domain. In certain embodiments, provided are CARs that comprise—from N-terminus to C-terminus—a variable heavy chain (V_{H}) polypeptide of an antibody described herein, a linker, a variable light chain (V_{L}) of an antibody described herein, a CD28 hinge region, a CD28 transmembrane domain, a 4-1 BB costimulatory domain, and a CD3ζ signaling domain. According to some embodiments, provided are CARs that comprise—from N-terminus to C-terminus—a variable light chain (V_{L}) polypeptide of an antibody described herein, a linker, a variable heavy chain (V_{H}) of an antibody described herein, a CD28 hinge region, a CD28 transmembrane domain, a 4-1 BB costimulatory domain, and a CD3C signaling domain. Any of the CARs of the present disclosure may include a domain N-terminal to the V_{H} polypeptide. For example, a leader sequence (e.g., a GM-CSFRalpha leader sequence) may be present at the N-terminus of a CAR of the present disclosure.

The amino acid sequences of non-limiting examples of anti-VV B5 CARs according to embodiments of the present disclosure are provided in Table 5 below, where the amino acid sequences of the CARs in Table 5 include an amino acid sequence of an N-terminal leader sequence (here, an N-terminal GM-CSFRalpha leader sequence). Any desired leader sequence (e.g., a GM-CSFRalpha leader sequence) may be present at the N-terminus of such CARs. As will be appreciated by one of ordinary skill in the art, the amino acid sequence of one or more of the domains indicated in Table 5 (e.g., linker, hinge, transmembrane, co-stimulatory, signaling, etc.) may be modified as desired, e.g., to improve the functionality, etc. of the CAR. In Table 5, segments/domains of the CARs are indicated by alternating underlining, and the identities of the segments/domains are provided in the left column.

TABLE 5
Example Anti-VV B5 CAR Amino Acid Sequences

| | |
|---|---|
| B5-CAR-050 | MLLLVTSLLLCELPHPAFLLIPEVQLLESGGGLVQPGGSLRLSCAAS |
| •GM-CSFRα Leader | GFSFSSSYYMCWVRQAPGKGLEWIACIYTSSGSAYYADSVKGRFT |
| •V_{H} (A048-H3) | ISRDNSKNTLYLQMNSLRAEDTAVYYCVRNAVGSSYYLYLWGQGT |
| •Linker | LVTVSSGSTSGSGKPGSGEGSTKGAQVLTQSPSTLSASVGDRVTI |
| •V_{L} (A048-L2) | TCQASQSVAGNNYLSWYQQKPGKAPKLLIYSVSTLASGVPSRFSG |
| •CD8 hinge | SGSGTEFTLTISSLQPDDFATYYCQGYYNDGIWAFGQGTKVEIKTT |
| •CD8 transmembrane | TPAPRPPTPAPTIASQPLSLRPEACRPAAGGAVHTRGLDFACDIYI |
| •4-1BB | WAPLAGTCGVLLLSLVITLYCKRGRKKLLYIFKQPFMRPVQTTQEE |
| •CD3ζ | DGCSCRFPEEEEGGCELRVKFSRSADAPAYQQGQNQLYNELNLG |
| (SEQ ID NO: 55) | RREEYDVLDKRRGRDPEMGGKPRRKNPQEGLYNELQKDKMAEAY |
| | SEIGMKGERRRGKGHDGLYQGLSTATKDTYDALHMQALPPR |
| B5-CAR-051 | MLLLVTSLLLCELPHPAFLLIPEVQLLESGGGLVQPGGSLRLSCAAS |
| •GM-CSFRα Leader | GFSFSSSYYMCWVRQAPGKGLEWIACIYTSSGSAYYANWAKGRF |
| •V_{H} (A048-H1) | TISRDNSKNTLYLQMNSLRAEDTAVYYCVRNAVGSSYYLYLWGQG |
| •Linker | TLVTVSSGSTSGSGKPGSGEGSTKGAQVLTQSPSSLSASVGDRVT |
| •V_{L} (A048-L4) | ITCQASQSVAGNNYLSWYQQKPGKVPKLLIYSVSTLASGVPSRFSG |
| •CD8 hinge | SGSGTDFTLTISSLQPEDVATYYCQGYYNDGIWAFGQGTKVEIKTT |
| •CD8 transmembrane | TPAPRPPTPAPTIASQPLSLRPEACRPAAGGAVHTRGLDFACDIYI |
| •4-1BB | WAPLAGTCGVLLLSLVITLYCKRGRKKLLYIFKQPFMRPVQTTQEE |
| •CD3ζ | DGCSCRFPEEEEGGCELRVKFSRSADAPAYQQGQNQLYNELNLG |
| (SEQ ID NO: 56) | RREEYDVLDKRRGRDPEMGGKPRRKNPQEGLYNELQKDKMAEAY |
| | SEIGMKGERRRGKGHDGLYQGLSTATKDTYDALHMQALPPR |

A CAR of the present disclosure may include one or more additional domains as desired. Non-limiting examples of such additional domains include a ribosomal skip element, an enzymatic domain (e.g., a domain having nuclease activity, e.g., restriction endonuclease activity), a domain that enables detection of the CAR (e.g., a reporter protein domain (e.g., a fluorescent protein (e.g., eGFP, mCherry, or the like), a luminescent protein, and/or the like)), etc. For example, in certain embodiments, provided are CARs that comprise a ribosomal skip element, a restriction enzyme domain, and/or a reporter protein domain.

According to some embodiments, a CAR of the present disclosure is provided by a single polypeptide. In certain embodiments, a CAR of the present disclosure is provided by two or more polypeptides. When the CAR is provided by two or more polypeptides, the CAR may be provided in any useful multi-polypeptide format, including universal CAR formats such as biotin-binding immune receptor (BBIR) format (see, e.g., Urbanska K, Powell D J. Development of a novel universal immune receptor for antigen targeting to infinity and beyond. Oncoimmunology. 2012; 1(5):777-779. doi:10.4161/onci.19730, and Urbanska K, Lanitis E, Poussin M, et al. A universal strategy for adoptive immunotherapy of cancer through use of a novel T cell antigen receptor. 2013; 72(7):1844-1852. doi: 10.1158/0008-5472. CAN-11-3890.A); a switchable CAR format with peptide NeoEpitope (PNE) (see, e.g., Kim et al. (2015) J Am Chem Soc. 2015; 137(8):2832-2835; Ma et al. (2016) Proc Natl Acad Sci 113(4):E450-8; Rodgers et al. (2016) Proc Natl Acad Sci. 113(4):E459-E468; Viaud et al. (2018) Proc Natl Acad Sci 115(46):E10898-E10906); a SUPRA CAR format with leucine zippers (see, e.g., Cho et al. (2108) Cell 173(6):1426-1438.e11); a CAR-T Adapter Molecule (CAM)-based format with FITC-folic acid (see, e.g., Lee et al. (2019) Cancer Res. 79(2):387-396; and Lu et al. (2019) Front Oncol. 9:151); anti-FITC-folic acid adaptor format (see, e.g., Chu et al. (2018) Biosci Trends. 12(3):298-308); anti-FITC antibody adaptor CAR format (see, e.g., Tamada et al. (2012) Clin Cancer Res. 18(23):6436-6445); Fc-targeting (e.g., anti-CD16) CAR+anti-tumor antibody format (see, e.g., Kudo et al. (2014) Cancer Res. 74(1):93-103); and the like.

### Conjugates

The present disclosure also provides conjugates. According to some embodiments, a conjugate of the present disclosure comprises any of the antibodies or fusion proteins of the present disclosure, and an agent conjugated to the antibody or fusion protein. The term “conjugated” generally refers to a chemical linkage, either covalent or non-covalent, usually covalent, that proximally associates one molecule of interest with a second molecule of interest. In certain embodiments, the agent conjugated to the antibody or fusion protein is selected from a chemotherapeutic agent, a toxin, a radiation-sensitizing agent, a radioactive isotope (e.g., a therapeutic radioactive isotope), a detectable label, and a half-life extending moiety.

According to some embodiments, the agent is a therapeutic agent, e.g., a chemotherapeutic agent. Therapeutic agents of interest include agents capable of affecting the function of a cell/tissue to which the conjugate binds via specific binding of the antibody portion of the conjugate to the antigen. When the function of the cell/tissue is pathological, an agent that reduces the function of the cell/tissue may be employed. In certain aspects, a conjugate of the present disclosure includes an agent that reduces the function of a target cell/tissue by inhibiting cell proliferation and/or killing the cell/tissue. Such agents may vary and include cytostatic agents and cytotoxic agents, e.g., an agent capable of killing a target cell tissue with or without being internalized into a target cell.

In certain embodiments, the therapeutic agent is a cytotoxic agent selected from an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof.

According to some embodiments, the agent is a toxin, such as a protein toxin selected from hemiasterlin and hemiasterlin analogs such as HTI-286 (e.g., see U.S. Pat. No. 7,579,323; WO 2004/026293; and U.S. Pat. No. 8,129,407, the full disclosures of which are incorporated herein by reference), abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which may be employed include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

In certain embodiments, the agent is a radiation-sensitizing agent. As used herein, a “radiation-sensitizing agent” is an agent that enhances the ability of radiation to kill tumor cells. Non-limiting examples of radiation-sensitizing agents that may be conjugated to the antibody or fusion protein include cisplatin, 5-fluorouracil (5-FU), AZD7762, selumetinib, and the like.

In certain embodiments, the agent is a radioisotope, e.g., useful for therapy and/or detection (e.g., imaging). Non-limiting examples of radioisotopes that may be conjugated to the antibody or fusion protein include but are not limited to ²²⁵Ac, ¹¹¹Ag, ¹¹⁴Ag, ⁷¹As, ⁷²As, ⁷⁷As, ²¹¹At, ¹⁹⁸Au, ¹⁹⁹Au, ²¹²Bi, ²¹³Bi, ⁷⁵Br, ⁷⁶Br, ¹¹C, ¹³C, ⁵⁵Co, ⁶²Cu, ⁶⁴Cu, ⁶⁷Cu, ¹⁶⁵Dy, ¹⁶⁶Dy, ¹⁶⁹Er, ¹⁸F, ¹⁹F, ⁵²Fe, ⁵⁹Fe, ⁶⁶Ga, ⁶⁷Ga, ⁶⁸Ga, ⁷²Ga, ¹⁵⁴⁻¹⁵⁸Gd, ¹⁵⁷Gd, ¹⁵⁹Gd, ¹⁶⁶Ho, ¹²⁰I, ¹²¹I, ¹²³I, ¹²⁴I, ¹²⁵I, ¹³¹I, ¹¹⁰In, ¹¹¹In, ^{113m}In, ¹⁹⁴Ir, ^{81m}Kr, ¹⁷⁷Lu, ⁵¹Mn, ⁵²Mn, ⁹⁹Mo, ¹³N, ¹⁵N, ¹⁵O, ¹⁷O, ³²P, ³³P, ²¹¹Pb, ²¹²Pb, ¹⁰⁹Pd, ¹⁴⁹Pm, ¹⁵¹Pm, ¹⁴²Pr, ¹⁴³Pr, ¹⁹¹PT, ^{193m}PT, ^{195m}Pt, ²²³Ra, ¹⁴²Rb, ¹⁸⁶Re, ¹⁸⁸Re, ¹⁸⁹Re, ¹⁰⁵Rh, ⁴⁷Sc, ⁷⁵Se, ¹⁵³Sm, ^{117m}Sn, ¹²¹Sn, ⁸³Sr, ⁸⁹Sr, ¹⁶¹Tb, ⁹⁴Tc, ⁹⁹Tc, ^{99m}Tc, ²²⁷Th, ²⁰¹Tl, ¹⁷²Tm, ¹²⁷Te, ⁹⁰Y, ¹⁶⁹Yb, ¹⁷⁵Yb, ¹³³X, and ⁸⁹Zr.

In certain embodiments, a radioisotope is conjugated to the antibody or fusion protein via a chelator, for example, a bifunctional chelator. A bifunctional chelator may contain a metal chelating moiety that binds the radioisotope in a stable coordination complex and a reactive functional group that is covalently linked to a targeting moiety, such as any of the antibodies or fusion proteins of the present disclosure, so that the radioisotope may be properly directed to the desirable molecular target in vivo. Non-limiting examples of bifunctional chelators that may be employed to conjugate an antibody or fusion protein of the present disclosure to a radioisotope include p-SCN-Bn-DOTA and p-SCN-Bn-deferoxamine. Additional examples of bifunctional chelators that may be employed to conjugate an antibody or fusion protein of the present disclosure to a radioisotope include those described in Price & Orvig (2014) Chem. Soc. Rev. 43:260; and Brechbiel (2008) Q J Nucl Med Mol Imaging 52(2):166-173.

According to some embodiments, the radioisotope is a therapeutic radioisotope. In certain embodiments, the radioisotope is an alpha emitting radioisotope, e.g., ²²⁵Ac, ²¹¹At, ²¹²Bi/²¹²Pb, ²¹³Bi, ²²³Ra, or ²²⁷Th. In other embodiments, the radioisotope is a beta minus emitting radioisotope, e.g., ³²P, ³³P, ⁶⁷Cu, ⁹⁰Y, ¹³¹I or ¹⁷⁷Lu.

According to some embodiments, the agent is a labeling agent. By “labeling agent” (or “detectable label”) is meant the agent detectably labels the antibody or fusion protein, such that the antibody or fusion protein may be detected in an application of interest (e.g., in vitro and/or in vivo research and/or clinical applications). Detectable labels of interest include radioisotopes (e.g., gamma or positron emitters), enzymes that generate a detectable product (e.g., horseradish peroxidase, alkaline phosphatase, luciferase, etc.), fluorescent proteins, paramagnetic atoms, and the like. In certain aspects, the antibody or fusion protein is conjugated to a specific binding partner of detectable label, e.g., conjugated to biotin such that detection may occur via a detectable label that includes avidin/streptavidin.

In certain embodiments, the agent is a labeling agent that finds use in in vivo imaging, such as near-infrared (NIR) optical imaging, single-photon emission computed tomography (SPECT)±CT imaging, positron emission tomography (PET)±CT imaging, nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain embodiments, the labeling agent is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 8000W Fluors. According to some embodiments, the labeling agent is an in vivo imaging agent that finds use in SPECT imaging applications, non-limiting examples of which include ^{99m}Tc, ¹¹¹In, ¹²³I, ²⁰¹Tl, and ¹³³Xe. In certain embodiments, the labeling agent is an in vivo imaging agent that finds use in PET imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, or the like.

For half-life extension, the antibodies and fusion proteins of the present disclosure may be conjugated to an agent that provides for an improved pharmacokinetic profile (e.g., by PEGylation, hyperglycosylation, and the like). Modifications that can enhance serum half-life are of interest. A subject antibody or fusion protein may be “PEGylated”, as containing one or more poly(ethylene glycol) (PEG) moieties. Methods and reagents suitable for PEGylation of a protein are well known in the art and may be found, e.g., in U.S. Pat. No. 5,849,860. PEG suitable for conjugation to a protein is generally soluble in water at room temperature and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject antibody or fusion protein can be linear. The PEG conjugated to the subject antibody or fusion protein may also be branched. Branched PEG derivatives such as those described in U.S. Pat. No. 5,643,575, “star-PEGs” and multi-armed PEGs. Star PEGs are described in the art including, e.g., in U.S. Pat. No. 6,046,305.

Where the subject antibody or fusion protein is to be isolated from a source, the antibody or fusion protein may be conjugated to one or more moieties that facilitate purification, such as members of specific binding pairs, e.g., biotin (member of biotin-avidin specific binding pair), a lectin, and the like. The antibody can also be bound to (e.g., immobilized onto) a solid support, including, but not limited to, polystyrene plates or beads, magnetic beads, test strips, membranes, and the like.

Where the antibodies or fusion proteins are to be detected in an assay, the antibodies or fusion proteins may contain a detectable label, e.g., a radioisotope (e.g., ⁸⁹Zr; ¹¹¹In, and the like), an enzyme which generates a detectable product (e.g., luciferase, β-galactosidase, horse radish peroxidase, alkaline phosphatase, and the like), a fluorescent protein, a chromogenic protein, dye (e.g., fluorescein isothiocyanate, rhodamine, phycoerythrin, and the like); fluorescence emitting metals, e.g., ¹⁵²Eu, or others of the lanthanide series, attached to the protein through metal chelating groups such as EDTA; chemiluminescent compounds, e.g., luminol, isoluminol, acridinium salts, and the like; bioluminescent compounds, e.g., luciferin; fluorescent proteins; and the like. Indirect labels include antibodies specific for a subject protein, wherein the antibody may be detected via a secondary antibody; and members of specific binding pairs, e.g., biotin-avidin, and the like.

Any of the above agents may be conjugated to the antibody or fusion protein via a linker. If present, the linker molecule(s) may be of sufficient length to permit the antibody or fusion protein and the linked agent to allow some flexible movement between the antibody or fusion protein and the linked agent. Linker molecules may be, e.g., about 6-50 atoms long. Linker molecules may also be, e.g., aryl acetylene, ethylene glycol oligomers containing 2-10 monomer units, diamines, diacids, amino acids, or combinations thereof.

Where the linkers are peptides, the linkers can be of any suitable length, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids in length.

Flexible linkers include glycine polymers (G)ₙ, glycine-serine polymers, glycine-alanine polymers, alanine-serine polymers, and other flexible linkers known in the art. Glycine and glycine-serine polymers may be used where relatively unstructured amino acids are of interest, and may serve as a neutral tether between components. The ordinarily skilled artisan will recognize that design of an antibody or fusion protein conjugated to any agents described above can include linkers that are all or partially flexible, such that the linker can include a flexible linker as well as one or more portions that confer a less flexible structure.

According to some embodiments, the antibody or fusion protein is conjugated to the agent via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the antibody is conjugated to the agent via a cleavable linker. According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13; Nolting, B. (2013) Methods Mol Biol. 1045:71-100; Tsuchikama and An (2018) Protein & Cell 9(1):33-46; and elsewhere.

Numerous strategies are available for linking agents to an antibody or fusion protein directly, or indirectly via a linker. For example, the agent may be derivatized by covalently attaching a linker to the agent, where the linker has a functional group capable of reacting with a “chemical handle” on the antibody or fusion protein. The functional group on the linker may vary and may be selected based on compatibility with the chemical handle on the antibody or fusion protein. According to one embodiment, the chemical handle on the antibody or fusion protein is provided by incorporation of an unnatural amino acid having the chemical handle into the antibody or fusion protein. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into an antibody of a conjugate of the present disclosure, which unnatural amino acid may be selected to provide a functional group of interest, are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere. An unnatural amino acid may be incorporated into an antibody or fusion protein via chemical synthesis or recombinant approaches, e.g., using a suitable orthogonal amino acyl tRNA synthetase-tRNA pair for incorporation of the unnatural amino acid during translation of the antibody or fusion protein in a host cell.

The functional group of an unnatural amino acid present in the antibody or fusion protein may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the antibody or fusion protein and the linker portion of a linker-agent moiety may include an alkyne functional group, such that the antibody or fusion protein and linker-agent moiety are covalently conjugated via azide-alkyne cycloaddition. Conjugation may be carried out using, e.g., a copper-catalyzed azide-alkyne cycloaddition reaction.

In certain embodiments, the chemical handle on the antibody or fusion protein does not involve an unnatural amino acid. An antibody containing no unnatural amino acids may be conjugated to the agent by utilizing, e.g., nucleophilic functional groups of the antibody or fusion protein (such as the N-terminal amine or the primary amine of lysine, or any other nucleophilic amino acid residue) as a nucleophile in a substitution reaction with a moiety bearing a reactive leaving group or other electrophilic group. An example would be to prepare an agent-linker moiety bearing an N-hydroxysuccinimidyl (NHS) ester and allow it to react with the antibody or fusion protein under aqueous conditions at elevated pH (˜10) or in polar organic solvents such as DMSO with an added non-nucleophilic base, such as N,N-diisopropylethylamine.

It will be appreciated that the particular approach for attaching a linker, agent and/or antibody or fusion protein to each other may vary depending upon the particular linker, agent and/or antibody or fusion protein and functional groups selected and employed for conjugating the various components to each other.

### Methods of Producing Antibodies

Using the information provided herein, the anti-VV B5 antibodies and fusion proteins of the present disclosure may be prepared using standard techniques known to those of skill in the art. For example, a nucleic acid sequence(s) encoding the amino acid sequence of an antibody or fusion protein of the present disclosure can be used to express the antibodies or fusion proteins. The polypeptide sequences provided herein (see, e.g., Tables 1, 3, 4 and 5) can be used to determine appropriate nucleic acid sequences encoding the antibodies or fusion proteins and the nucleic acids sequences then used to express one or more antibodies or fusion proteins specific for VV B5. The nucleic acid sequence(s) can be optimized to reflect particular codon “preferences” for various expression systems according to standard methods well known to those of skill in the art. Using the sequence information provided, the nucleic acids may be synthesized according to a number of standard methods known to those of skill in the art.

Once a nucleic acid(s) encoding a subject antibody or fusion protein is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the antibodies and fusion proteins of the present disclosure can be achieved by operably linking a nucleic acid encoding the antibody or fusion protein to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing antibodies are available using, for example, E. coli, Bacillus sp. and Salmonella. E. coli systems may also be used.

The antibody gene(s) may also be subcloned into an expression vector that allows for the addition of a tag (e.g., FLAG, hexahistidine, and the like) at the C-terminal end or the N-terminal end of the antibody (e.g., IgG, Fab, scFv, etc.) to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject antibody or fusion protein is isolated and cloned, one can express the nucleic acid in a variety of recombinantly engineered cells known to those of skill in the art. Examples of such cells include bacteria, yeast, filamentous fungi, insect (e.g. those employing baculoviral vectors), and mammalian cells.

Isolation and purification of a subject antibody or fusion protein can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound antibody. The isolated antibody or fusion protein can be further purified by dialysis and other methods normally employed in protein purification methods. In one embodiment, the antibody may be isolated using metal chelate chromatography methods. Antibodies and fusion proteins of the present disclosure may contain modifications to facilitate isolation, as discussed above.

The antibodies and fusion proteins may be prepared in substantially pure or isolated form (e.g., free from other polypeptides). The protein can be present in a composition that is enriched for the polypeptide relative to other components that may be present (e.g., other polypeptides or other host cell components). Purified antibodies and fusion proteins may be provided such that the antibody or fusion protein is present in a composition that is substantially free of other expressed proteins, e.g., less than 90%, usually less than 60% and more usually less than 50% of the composition is made up of other expressed proteins.

The antibodies and fusion proteins produced by prokaryotic cells may require exposure to chaotropic agents for proper folding. During purification from E. coli, for example, the expressed protein can be optionally denatured and then renatured. This can be accomplished, e.g., by solubilizing the bacterially produced antibodies and fusion proteins in a chaotropic agent such as guanidine HCl. The antibody or fusion protein is then renatured, either by slow dialysis or by gel filtration. Alternatively, nucleic acid encoding the antibodies and fusion proteins may be operably linked to a secretion signal sequence such as pelB so that the antibodies and fusion proteins are secreted into the periplasm in correctly-folded form.

The present disclosure also provides cells that produce the antibodies and fusion proteins of the present disclosure, where suitable cells include eukaryotic cells, e.g., mammalian cells. For example, the present disclosure provides a recombinant host cell (also referred to herein as a “genetically modified host cell”) that is genetically modified with one or more nucleic acids comprising a nucleotide sequence encoding a heavy and/or light chain of an antibody of the present disclosure.

Techniques for creating recombinant DNA versions of the antigen-binding regions of antibody molecules which bypass the generation of hybridomas are also contemplated herein. DNA is cloned into a bacterial (e.g., bacteriophage), yeast (e.g. Saccharomyces or Pichia), insect or mammalian expression system, for example. One example of a suitable technique uses a bacteriophage lambda vector system having a leader sequence that causes the expressed antibody (e.g., Fab or scFv) to migrate to the periplasmic space (between the bacterial cell membrane and the cell wall) or to be secreted. One can rapidly generate great numbers of functional fragments (e.g., Fab or scFv) for those which bind the antigen of interest.

The antibodies and fusion proteins that specifically bind VV B5 can be prepared using a wide variety of techniques known in the art including the use of recombinant, phage display technologies, Selected Lymphocyte Antibody Method (SLAM), or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds VV B5 can be selected or identified with VV B5, e.g., using labeled VV B5 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene Ill or gene VIII protein. The production of high affinity human antibodies by chain shuffling is known, as are combinatorial infection and in vivo recombination as a strategy for constructing large phage libraries. In another embodiment, ribosomal display can be used to replace bacteriophage as the display platform. Cell surface libraries may be screened for antibodies. Such procedures provide alternatives to traditional hybridoma techniques for the isolation and subsequent cloning of monoclonal antibodies.

After phage selection, the antibody coding regions from the phage can be isolated and used to generate whole antibodies, or any desired antibody fragments, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

### Nucleic Acids, Expression Vectors and Cells

In view of the section above regarding methods of producing the antibodies and fusion proteins of the present disclosure, it will be appreciated that the present disclosure also provides nucleic acids, expression vectors and cells.

In certain embodiments, provided is a nucleic acid encoding a variable heavy chain (V_{H}) polypeptide, a variable light chain (V_{L}) polypeptide, or both, of an anti-VV B5 antibody or fusion protein of the present disclosure, e.g., any of such antibodies and fusion proteins (e.g., CARs) described hereinabove. According to some embodiments, the antibody is a single chain antibody (e.g., an scFv), and the nucleic acid encodes the single chain antibody. In certain embodiments, provided is a nucleic acid that encodes a CAR of the present disclosure, e.g., a CAR comprising: a single chain antibody comprising a V_{H} polypeptide and a V_{L} polypeptide of an anti-VV B5 antibody of the present disclosure; a transmembrane domain; and an intracellular signaling domain. Examples of such single chain antibodies, transmembrane domains, and intracellular signaling domains are described in detail above.

Also provided are expression vectors comprising any of the nucleic acids of the present disclosure. Expression of natural or synthetic nucleic acids encoding the anti-VV B5 antibodies and fusion proteins of the present disclosure can be achieved by operably linking a nucleic acid encoding the antibody or fusion protein to a promoter (which is either constitutive or inducible) and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

Cells that comprise any of the nucleic acids and/or expression vectors of the present disclosure are also provided. According to some embodiments, a cell of the present disclosure comprises a nucleic acid that encodes the V_{H} polypeptide of the antibody and the V_{L} polypeptide of the antibody. In certain such embodiments, the antibody is a single chain antibody (e.g., an scFv), and the nucleic acid encodes the single chain antibody. According to some embodiments, provided is a cell comprising a first nucleic acid encoding a variable heavy chain (V_{H}) polypeptide of an antibody of the present disclosure, and a second nucleic acid encoding a variable light chain (V_{L}) polypeptide of the antibody. In certain embodiments, such as cell comprises a first expression vector comprising the first nucleic acid, and a second expression vector comprising the second nucleic acid.

Also provided are methods of making an anti-VV B5 antibody or fusion protein of the present disclosure, the methods including culturing a cell of the present disclosure under conditions suitable for the cell to express the anti-VV B5 or fusion protein, wherein the antibody or fusion protein is produced. The conditions for culturing the cell such that the antibody or fusion protein is expressed may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO2, e.g., 3% to 10%, such as 5%).

### Compositions

As summarized above, the present disclosure also provides compositions. According to some embodiments, a composition of the present disclosure includes an anti-VV B5 antibody, fusion protein, or conjugate of the present disclosure. For example, the antibody, fusion protein, or conjugate may be any of the antibodies, fusion proteins (e.g., CARs), or conjugates described in the Anti-VV B5 Antibodies section hereinabove, which descriptions are incorporated but not reiterated herein for purposes of brevity.

In certain aspects, a composition of the present disclosure includes the antibody, fusion protein, or conjugate present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure includes an anti-VV B5 antibody of the present disclosure (or conjugate or fusion protein comprising same), and a pharmaceutically acceptable carrier.

The anti-VV B5 antibodies, fusion proteins (e.g., CARs), or conjugates can be incorporated into a variety of formulations for therapeutic administration. More particularly, the anti-VV B5 antibodies, fusion proteins, or conjugates can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the antibodies, fusion proteins, or conjugates for administration to an individual (e.g., suitable for human administration) are generally sterile and may further be free of detectable pyrogens or other contaminants contraindicated for administration to a patient according to a selected route of administration.

In pharmaceutical dosage forms, the antibodies, fusion proteins, or conjugates can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the antibodies, fusion proteins, or conjugates can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The antibodies, fusion proteins, or conjugates can be formulated for parenteral (e.g., intravenous, intra-arterial, intraosseous, intramuscular, intracerebral, intracerebroventricular, intrathecal, subcutaneous, etc.) administration. In certain aspects, the antibodies, fusion proteins, or conjugates are formulated for injection by dissolving, suspending or emulsifying the antibodies, fusion proteins, or conjugates in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the antibodies, fusion proteins, or conjugates may be prepared by mixing the antibodies, fusion proteins, or conjugates having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the antibodies, fusion proteins, or conjugates may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the antibody and/or T cell activator against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the antibody, fusion protein, or conjugate, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

Aspects of the present disclosure further include kits. In certain embodiments, the kits find use in practicing the methods of the present disclosure, e.g., methods comprising administering a pharmaceutical composition of the present disclosure to an individual to target the anti-VV B5 antibody, fusion protein (e.g., CAR) or conjugate to vaccinia virus-infected cells (including but not limited to, cancer cells) in the individual.

Accordingly, in certain embodiments, a kit of the present disclosure comprises any of the pharmaceutical compositions of the present disclosure, and instructions for administering the pharmaceutical composition to an individual in need thereof. The pharmaceutical composition included in the kit may include any of the anti-VV B5 antibodies, fusion proteins, and/or conjugates of the present disclosure, e.g., any of the antibodies, fusion proteins, and/or conjugates described hereinabove. As will be appreciated, the kits of the present disclosure may include any of the agents and features described above in the sections relating to the subject anti-VV B5 antibodies, fusion proteins, conjugates and compositions, which are not reiterated herein for purposes of brevity.

The kits of the present disclosure may include a quantity of the compositions, present in unit dosages, e.g., ampoules, or a multi-dosage format. As such, in certain embodiments, the kits may include one or more (e.g., two or more) unit dosages (e.g., ampoules) of a composition that includes an anti-VV B5 antibody, fusion protein, and/or conjugate of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular antibody, fusion protein, and/or conjugate employed, the effect to be achieved, and the pharmacodynamics associated with the antibody, fusion protein, and/or conjugate, in the individual. In yet other embodiments, the kits may include a single multi dosage amount of the composition.

In certain embodiments, a kit of the present disclosure includes instructions for targeting the anti-VV B5 antibody, fusion protein or conjugate present in the pharmaceutical composition to VV-infected cells in an individual. According to some embodiments, the infected cells are VV-infected cancer cells in an individual having cancer (e.g., to treat the cancer of the individual), e.g., by administering the pharmaceutical composition to the individual, wherein the individual comprises cancer cells infected with VV, and wherein the anti-VV B5 antibody, fusion protein (e.g., CARs) or conjugate is targeted to the infected cancer cells by VV antigens expressed on the surface of the infected cancer cells.

According to some embodiments, a kit of the present disclosure may further include pharmaceutical composition comprising VV (e.g., JX-594, GL-ONC1, a strain of VV selected from Western Reserve, Wyeth, Lister, Copenhagen, Temple of Heaven, Patwadangar, and Modified Vaccinia Virus Ankara, etc.). Such a kit may further include instructions for administering to an individual having cancer the pharmaceutical composition comprising VV in an amount effective to infect cells in the individual (e.g., infect cancer cells in an individual having cancer), e.g., prior to administration of a pharmaceutical composition comprising an anti-VV B5 antibody, fusion protein or conjugate of the present disclosure.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods

Aspects of the present disclosure further include methods of using the anti-VV B5 antibodies, fusion proteins (e.g., CARs), and conjugates of the present disclosure. The methods are useful in a variety of contexts, including in vitro and/or in vivo research and/or clinical applications.

In certain aspects, provided are methods that comprise administering an effective amount of a pharmaceutical composition comprising any of the anti-VV B5 antibodies of the present disclosure (including any of the fusion proteins or conjugates comprising such antibodies) to an individual, where the individual comprises cells infected with VV that encode the VV B5 antigen to which the antibodies bind, and where the anti-VV B5 antibody, fusion protein or conjugate is targeted to the infected cells by VV B5 antigens expressed on the surface of the infected cells. According to some embodiments, such methods further comprise, prior to administering the pharmaceutical composition to the individual, infecting the cells by administering an effective amount of the VV to the individual.

When bound on the infected cell surface (e.g., infected cancer cell surface), the anti-VV B5 antibody (or fusion protein or conjugate comprising same) may induce cytotoxicity, e.g., via antibody-dependent cellular cytotoxicity (ADCC), by recruiting complement in complement dependent cytotoxicity (CDC), via antibody-dependent cellular phagocytosis (ADCP), via epitope spreading, or by some other mechanism. The antibodies may be modified in the Fc region to provide desired or enhanced effector functions. This may be achieved by introducing one or more amino acid substitutions in an Fc region of the antibody. Alternatively, where it is desirable to eliminate or reduce effector function, so as to minimize side effects or therapeutic complications, certain other Fc regions may be used.

According to some embodiments, provided are methods that comprise administering an effective amount of a pharmaceutical composition comprising any of the anti-VV B5 antibodies of the present disclosure (including any of the fusion proteins or conjugates comprising such antibodies) to an individual, where the individual comprises cancer cells infected with VV that encode the VV B5 antigen to which the antibodies bind, and where the anti-VV B5 antibody, fusion protein or conjugate is targeted to the infected cancer cells by VV B5 antigens expressed on the surface of the infected cancer cells. According to some embodiments, such methods further comprise, prior to administering the pharmaceutical composition to the individual, infecting the cancer cells by administering an effective amount of the VV to the individual. Such methods find use, e.g., in treating the cancer of the individual.

In certain embodiments, the pharmaceutical composition comprises any of the anti-VV B5 antibody conjugates of the present disclosure. For example, the pharmaceutical composition may comprise a conjugate, where the anti-VV B5 antibody is conjugated to a detectable label or radioactive isotope which is an in vivo imaging agent. Such methods may further comprise imaging the infected cells (e.g., infected cancer cells) in the individual using the in vivo imaging agent. The methods in which a conjugate comprising a detectable label or radioactive isotope is administered to the individual find use in imaging the cells (e.g., cancer cells) in the individual, e.g., for diagnostic, prognostic, and/or therapy (e.g., anti-cancer therapy) monitoring purposes.

According to some embodiments, the pharmaceutical composition may comprise a conjugate of the present disclosure, where the anti-VV B5 antibody is conjugated to an agent selected from a chemotherapeutic agent, a toxin, a radiation sensitizing agent, a therapeutic radioactive isotope, and a radioisotope that permits in vivo imaging of the antibody. The agent may be any such agents described in the Conjugates section above.

In certain embodiments, provided are methods of targeting a CAR that specifically binds an VV B5 antigen to VV-infected cells (e.g., VV-infected cancer cells) in an individual. Such methods comprise administering to the individual an effective amount of a pharmaceutical composition comprising a CAR of the present disclosure, where the target cells in the individual are infected with VV and express the VV B5 antigen on their surface. The CAR may be expressed on the surface of a cell, e.g., an immune cell, such as an immune effector cell, e.g., a T cell, an NK cell, an NKT cell, a macrophage, or the like. For example, the CAR may be present on the surface of T cells, where the method is a method of targeting CAR T cells to the infected cells (e.g., cancer cells) in the individual. According to some embodiments, such methods further comprise, prior to administering the pharmaceutical composition to the individual, infecting the cells (e.g., cancer cells) by administering an effective amount of the OV to the individual. Such methods find use, e.g., in treating the cancer of the individual.

A pharmaceutical composition comprising cells that express a CAR on their surface may be prepared by a variety of methods. In some embodiments, a cell of the present disclosure is produced by transfecting the cell with a viral vector encoding the CAR. In some embodiments, the cell is a T cell, such that provided are methods of producing a CAR T cell. In some embodiments, such methods include activating a population of T cells (e.g., T cells obtained from an individual to whom a CAR T cell therapy will be administered), stimulating the population of T cells to proliferate, and transducing the T cell with a viral vector encoding the CAR. In some embodiments, the T cells are transduced with a retroviral vector, e.g., a gamma retroviral vector, encoding the CAR. In some embodiments, the T cells are transduced with a lentiviral vector encoding the CAR.

Cells of the present disclosure may be autologous/autogeneic (“self”) or non-autologous (“non-self,” e.g., allogeneic, syngeneic or xenogeneic). “Autologous” as used herein, refers to cells from the same individual. “Allogeneic” as used herein refers to cells of the same species that differ genetically from the cell in comparison. “Syngeneic,” as used herein, refers to cells of a different individual that are genetically identical to the cell in comparison. In some embodiments, the cells are T cells obtained from a mammal. In some embodiments, the mammal is a primate. In some embodiments, the primate is a human.

T cells may be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In certain embodiments, T cells can be obtained from a unit of blood collected from an individual using any number of known techniques such as sedimentation, e.g., FICOLL™ separation.

In some embodiments, an isolated or purified population of T cells is used. In some embodiments, T_{CTL} and T_{H} lymphocytes are purified from PBMCs. In some embodiments, the T_{CTL} and T_{H} lymphocytes are sorted into naïve (T_{N}), memory (T_{MEM}), and effector (T_{EFF}) T cell subpopulations either before or after activation, expansion, and/or genetic modification. Suitable approaches for such sorting are known and include, e.g., magnetic-activated cell sorting (MACS), where TN are CD45RA⁺CD62L⁺CD95⁻; TSCM are CD45RA⁺CD62L⁺CD95⁺; TCM are CD45RO⁺ CD62L⁺CD95⁺; and TEM are CD45RO⁺ CD62L⁻ CD95⁺. An example approach for such sorting is described in Wang et al. (2016) Blood 127(24):2980-90.

In some embodiments, a specific subpopulation of T cells expressing one or more of the following markers: CD3, CD4, CD8, CD28, CD45RA, CD45RO, CD62, CD127, and HLA-DR can be further isolated by positive or negative selection techniques. In some embodiments, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In some embodiments, the manufactured T cell compositions do not express one or more of the following markers: CD57, CD244, CD 160, PD-1, CTLA4, TIM3, and LAG3. In some embodiments, the manufactured T cell compositions do not substantially express one or more of the following markers: CD57, CD244, CD 160, PD-1, CTLA4, TIM3, and LAG3.

In order to achieve therapeutically effective doses of T cell compositions, the T cells may be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes. In some embodiments, T cells are activated and expanded for about 1 to 21 days, e.g., about 5 to 21 days.

In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of a nucleic acid (e.g., expression vector) encoding the CAR into the T cells.

In some embodiments, T cells are activated and expanded for about 6 hours, about 12 hours, about 18 hours or about 24 hours prior to introduction of a nucleic acid (e.g., expression vector) encoding the CAR into the T cells. In some embodiments, T cells are activated at the same time that a nucleic acid (e.g., an expression vector) encoding the CAR is introduced into the T cells.

In some embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

In some embodiments, the nucleic acid (e.g., an expression vector) encoding the CAR is introduced into the cell (e.g., a T cell) by microinjection, transfection, lipofection, heat-shock, electroporation, transduction, gene gun, microinjection, DEAE-dextran-mediated transfer, and the like. In some embodiments, the nucleic acid (e.g., expression vector) encoding the CAR is introduced into the cell (e.g., a T cell) by AAV transduction. The AAV vector may comprise ITRs from AAV2, and a serotype from any one of AAV1, AAV2, AAV3, AAV4, AAV5, AAV6, AAV7, AAV8, AAV9, or AAV 10. In some embodiments, the AAV vector comprises ITRs from AAV2 and a serotype from AAV6. In some embodiments, the nucleic acid (e.g., expression vector) encoding the CAR is introduced into the cell (e.g., a T cell) by lentiviral or retroviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BlV), or simian immunodeficiency virus (SIV). The lentiviral vector may be integration competent or an integrase deficient lentiviral vector (TDLV). In one embodiment, IDLV vectors including an HIV-based vector backbone (i.e., HIV cis-acting sequence elements) are employed.

In certain aspects, provided are methods of targeting a conjugate that specifically binds a VV B5 antigen to VV-infected cells (e.g., VV-infected cancer cells) in an individual. Such methods comprise administering to the individual an effective amount of a pharmaceutical composition comprising a conjugate comprising an anti-VV B5 antibody, where the target cells (e.g., cancer cells) in the individual are infected with VV and express the VV B5 antigen on their surface. According to some embodiments, such methods further comprise, prior to administering the pharmaceutical composition to the individual, infecting the cells (e.g., cancer cells) by administering an effective amount of the VV to the individual. Such methods find use, e.g., in treating the cancer of the individual.

In certain aspects, provided are methods comprising administering a pharmaceutical composition comprising cells (e.g., cancer cells) infected with VV. The pharmaceutical composition may further include an anti-VV B5 antibody, conjugate, or fusion protein of the present disclosure that specifically binds VV B5 antigen expressed by the infected cells, e.g., cancer cells. The cells (e.g., cancer cells) may have been removed from the individual during surgery. The cells (e.g., cancer cells) may have been altered (and killed) in the lab to make them more likely to be attacked by the immune system when administered back into the patient. The patient's immune system then attacks the cells and any similar cells still in the body. The antibody, conjugate, or fusion protein of the present disclosure may be employed according to this approach to promote uptake of tumor particles/antigens by Fc receptors on professional APC, leading to an enhanced immune response against the tumor.

The pharmaceutical compositions may be administered to any of a variety of individuals. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain aspects, the individual is an animal model (e.g., a mouse model, a primate model, or the like) of a cellular proliferative disorder, e.g., cancer.

The individual in need thereof may have a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the individual has cancer. The subject methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, breast cancer, squamous non-small cell lung cancer, rhabdomyosarcoma, metastatic renal cell carcinoma, basal cell carcinoma (basal cell epithelioma), and gliosarcoma. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

The anti-VV B5 antibodies, fusion proteins and conjugates of the present disclosure may be administered via a route of administration selected from oral (e.g., in tablet form, capsule form, liquid form, or the like), parenteral (e.g., by intravenous, intra-arterial, subcutaneous, intramuscular, or epidural injection), topical, intra-nasal, or intra-tumoral administration.

The anti-VV B5 antibodies, fusion proteins and conjugates of the present disclosure may be administered in a pharmaceutical composition in a therapeutically effective amount. By “therapeutically effective amount” is meant a dosage sufficient to produce a desired result, e.g., an amount sufficient to effect beneficial or desired therapeutic (including preventative) results, such as a reduction in a symptom, as compared to a control. With respect to cancer, in some embodiments, the therapeutically effective amount is sufficient to slow the growth of a tumor, reduce the size of a tumor, and/or the like. An effective amount can be administered in one or more administrations.

As described above, aspects of the present disclosure include methods for treating an individual, e.g., a cancer of an individual. By treatment is meant at least an amelioration of one or more symptoms associated with the medical condition (e.g., cancer) of the individual, where amelioration is used in a broad sense to refer to at least a reduction in the magnitude of a parameter, e.g. symptom, associated with the medical condition (e.g., cancer) being treated. As such, treatment also includes situations where the medical condition (e.g., cancer), or at least one or more symptoms associated therewith, are completely inhibited, e.g., prevented from happening, or stopped, e.g., terminated, such that the individual no longer suffers from the medical condition (e.g., cancer), or at least the symptoms that characterize the medical condition (e.g., cancer).

An anti-VV B5 antibody, fusion protein, or conjugate of the present disclosure may be administered to the individual alone or in combination with a second agent. Second agents of interest include, but are not limited to, agents approved by the United States Food and Drug Administration and/or the European Medicines Agency (EMA) for use in treating cancer. In some embodiments, the second agent is an immune checkpoint inhibitor. Immune checkpoint inhibitors of interest include, but are not limited to, a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When an antibody, fusion protein, or conjugate of the present disclosure is administered with a second agent, the antibody, fusion protein, or conjugate and the second agent may be administered to the individual according to any suitable administration regimen. According to certain embodiments, the antibody, fusion protein, or conjugate and the second agent are administered according to a dosing regimen approved for individual use. In some embodiments, the administration of the antibody, fusion protein, or conjugate permits the second agent to be administered according to a dosing regimen that involves one or more lower and/or less frequent doses, and/or a reduced number of cycles as compared with that utilized when the second agent is administered without administration of the antibody, fusion protein, or conjugate. In certain aspects, the administration of the second agent permits the antibody, fusion protein, or conjugate to be administered according to a dosing regimen that involves one or more lower and/or less frequent doses, and/or a reduced number of cycles as compared with that utilized when the antibody, fusion protein, or conjugate is administered without administration of the second agent.

In some embodiments, one or more doses of the antibody, fusion protein, or conjugate and the second agent are administered concurrently to the individual. By “concurrently” is meant the antibody, fusion protein, or conjugate and the second agent are either present in the same pharmaceutical composition, or the antibody, fusion protein, or conjugate and the second agent are administered as separate pharmaceutical compositions within 1 hour or less, 30 minutes or less, or 15 minutes or less.

In some embodiments, one or more doses of the antibody, fusion protein, or conjugate and the second agent are administered sequentially to the individual.

In some embodiments, the antibody, fusion protein, or conjugate and the second agent are administered to the individual in different compositions and/or at different times. For example, the antibody, fusion protein, or conjugate may be administered prior to administration of the second agent, e.g., in a particular cycle. Alternatively, the second agent may be administered prior to administration of the antibody, fusion protein, or conjugate, e.g., in a particular cycle. The second agent to be administered may be administered a period of time that starts at least 1 hour, 3 hours, 6 hours, 12 hours, 24 hours, 48 hours, 72 hours, or up to 5 days or more after the administration of the first agent to be administered.

In one example, the second agent is administered to the individual for a desirable period of time prior to administration of the antibody, fusion protein, or conjugate. In certain aspects, such a regimen “primes” the cancer cells to potentiate the anti-cancer effect of the antibody, fusion protein, or conjugate. Such a period of time separating a step of administering the second agent from a step of administering the antibody, fusion protein, or conjugate is of sufficient length to permit priming of the cancer cells, desirably so that the anti-cancer effect of the antibody, fusion protein, or conjugate is increased.

In some embodiments, administration of one agent is specifically timed relative to administration of the other agent. For example, in some embodiments, the antibody, fusion protein, or conjugate is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular individual of interest.

In some embodiments, the antibody, fusion protein, or conjugate and the second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of a second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between at least one pair of doses of the first agent. In some embodiments, different doses of the same agent are separated by a common interval of time; in some embodiments, the interval of time between different doses of the same agent varies. In certain aspects, different doses of the antibody, fusion protein, or conjugate and the second agent are separated from one another by a common interval of time; in some embodiments, different doses of the different agents are separated from one another by different intervals of time.

One exemplary protocol for interdigitating two intermittent, cycled dosing regimens may include: (a) a first dosing period during which a therapeutically effective amount the antibody, fusion protein, or conjugate is administered to the individual; (b) a first resting period; (c) a second dosing period during which a therapeutically effective amount of the second agent is administered to the individual; and (d) a second resting period. A second exemplary protocol for interdigitating two intermittent, cycled dosing regimens may include: (a) a first dosing period during which a therapeutically effective amount the second agent is administered to the individual; (b) a first resting period; (c) a second dosing period during which a therapeutically effective amount of the antibody, fusion protein, or conjugate is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include type and/or stage of a cancer against which the therapy is administered; properties (e.g., pharmacokinetic properties) of the antibody, fusion protein, or conjugate, and/or one or more features of the patient's response to therapy with the antibody, fusion protein, or conjugate. In some embodiments, length of one or both resting periods may be adjusted in light of pharmacokinetic properties (e.g., as assessed via plasma concentration levels) of one or the other of the administered agents. For example, a relevant resting period might be deemed to be completed when plasma concentration of the relevant agent is below a pre-determined level, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The antibody, fusion protein, or conjugate and the second agent may be administered together or independently via any suitable route of administration. The antibody, fusion protein, or conjugate and the second agent may be administered via a route of administration independently selected from oral, parenteral (e.g., by intravenous, intra-arterial, subcutaneous, intramuscular, or epidural injection), topical, or intra-nasal administration. According to certain embodiments, antibody, fusion protein, or conjugate and the second agent are both administered orally (e.g., in tablet form, capsule form, liquid form, or the like) either concurrently (in the same pharmaceutical composition or separate pharmaceutical compositions) or sequentially.

When the methods further comprise infecting the target cells (e.g., cancer cells) by administering the VV to the individual, any suitable administration regimen may be employed to infect the cancer cells. In certain embodiments, such methods comprise administering the VV and the pharmaceutical composition concurrently to the individual. Concurrent administration may take a variety of forms and encompasses administering, as separate compositions, a first composition comprising the VV and the pharmaceutical composition comprising the antibody, fusion protein, or conjugate. According to some embodiments, concurrent administration comprises administering, present in the same composition, the VV and the antibody, fusion protein, or conjugate. In one non-limiting example, a cell expressing a fusion protein (e.g., a CAR T cell of the present disclosure) is administered concurrently with the VV, where concurrent administration comprises administering the cells infected with the VV to the individual. By way of example, CAR T cells of the present disclosure infected with the VV may be administered to the individual to effect concurrent administration of the VV and CAR T cells to the individual.

In certain embodiments, when the methods further comprise infecting the target cells (e.g., cancer cells) by administering the VV to the individual, the target cells are infected by administering the VV to the individual prior to administering the pharmaceutical composition to the individual.

Any suitable approach may be employed to infect the target cells (e.g., cancer cells) in the individual. Poxvirus replication takes place in the cytoplasm, as the virus is sufficiently complex to have acquired all the functions necessary for genome replication. Once in the cell cytoplasm, gene expression is carried out by viral enzymes associated with the core. Expression is divided into 2 phases: early genes: which represent about of 50% genome, and are expressed before genome replication, and late genes, which are expressed after genome replication. The temporal control of expression is provided by the late promoters, which are dependent on DNA replication for activity. Genome replication is believed to involve self-priming, leading to the formation of high molecular weight concatemers, which are subsequently cleaved and repaired to make virus genomes. Viral assembly occurs in the cytoskeleton and probably involves interactions with the cytoskeletal proteins (e.g., actin-binding proteins). Inclusions form in the cytoplasm that mature into virus particles. Vaccinia virus is unique among DNA viruses as it replicates only in the cytoplasm of the host cell. Therefore, the large genome is required to code for various enzymes and proteins needed for viral DNA replication. During replication, vaccinia produces several infectious forms, which differ in their outer membranes: the intracellular mature virion (IMV), the intracellular enveloped virion (IEV), the cell-associated enveloped virion (CEV), and the extracellular enveloped virion (EEV).

To infect the target cells (e.g., cancer cells) with the VV, the VV is administered using a suitable route of administration. The route of administration may vary with the location and nature of the cancer, and may include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration and formulation. Intratumoral injection, or injection directly into the tumor vasculature is specifically contemplated for discrete, solid, accessible tumors. Local, regional or systemic administration also may be appropriate. The viral particles may advantageously be contacted by administering multiple injections to the tumor, spaced, for example, at approximately 1 cm intervals. Continuous administration also may be applied where appropriate, for example, by implanting a catheter into a tumor or into tumor vasculature. Such continuous perfusion may take place, for example, for a period of from about 1-2 hours, to about 2-6 hours, to about 6-12 hours, or about 12-24 hours following the initiation of administration. Administration regimens may vary, and often depend on tumor type, tumor location, disease progression, and health and age of the patient. Certain types of tumor will require more aggressive treatment, while at the same time, certain patients cannot tolerate more taxing protocols. The clinician will be best suited to make such decisions.

Injection of nucleic acid constructs may be delivered by syringe or any other method used for injection of a solution, so long as the expression construct can pass through the particular gauge of needle required for injection. An exemplary needleless injection system that may be used for the administration of VV is exemplified in U.S. Pat. No. 5,846,233. This system features a nozzle defining an ampule chamber for holding the solution and an energy device for pushing the solution out of the nozzle to the site of delivery. Another exemplary syringe system is one that permits multiple injections of predetermined quantities of a solution precisely at any depth (U.S. Pat. No. 5,846,225). Mixtures of VV particles or nucleic acids encoding same may be prepared in water suitably mixed with one or more excipients, carriers, or diluents. Dispersions may also be prepared in glycerol, liquid polyethylene glycols, and mixtures thereof and in oils.

A physician may start prescribing doses of VV vector at levels lower than that required in order to achieve the desired therapeutic effect and gradually increase the dosage until the desired effect is achieved. Alternatively, a physician may begin a treatment regimen by administering a dose of VV vector and subsequently administer progressively lower doses until a therapeutic effect is achieved, e.g., a reduction in the volume of one or more tumors.

Notwithstanding the appended claims, the present disclosure is also defined by the following embodiments.
1. An antibody that specifically binds to Vaccinia Virus B5 antigen (VV B5), wherein the antibody comprises:
a variable heavy chain (V_{H}) polypeptide comprising 70% or greater sequence identity to the amino acid sequence set forth in SEQ ID NO: 1, wherein the V_{H} polypeptide comprises:
   one or more mutations selected from the group consisting of: Q1E, E2V, Q3T, E5L/K, G9P, G10V, K13Q, E15G/T, G16E, S17T, T19R, T21S, T23A, A41P, R44K, G45A, T74D, S75N/T, S76_T77insK, T77N/S, T78Q, V79L, T80Y/V, Q82T, T84N, R85S/N, L86M, T87R/D, A88P, A89E/V, T93V, F95Y, P112Q, and any combination thereof, wherein numbering is according to SEQ ID NO:1;
   a V_{H} CDR1 comprising the amino acid sequence SSYYMC (SEQ ID NO:13),
   a V_{H} CDR2 comprising the amino acid sequence CIYTSSGSAYYA(N/D)(W/S)(A/V)KG (SEQ ID NO:14), and
   a V_{H} CDR3 comprising the amino acid sequence NAVGSSYYLYL (SEQ ID NO:17); and
a variable light chain (V_{L}) polypeptide comprising 70% or greater sequence identity to the amino acid sequence set forth in SEQ ID NO: 7, wherein the V_{L} polypeptide comprises:
   one or more mutations selected from the group consisting of: A1D/E, Q2I, V3Q, L4M, T7S, S9A, P10T/S, V11L, A13L, A14S, V15P, G17D/E, T18R, V19A, I21L, S22T, Q44K, P45A/V, N47K/R, V60I, S62A, K65S, Q72E/D, D79S, E81Q, C82P, D83E, A85F/V, T87V, G103Q, E106K, V107L, V108E, V109I, and any combination thereof, wherein numbering is according to SEQ ID NO: 7;
   a V_{L} CDR1 comprising the amino acid sequence QASQSVAGNNYLS (SEQ ID NO:18),
   a V_{L} CDR2 comprising the amino acid sequence SVSTLAS (SEQ ID NO:19), and
   a V_{L} CDR3 comprising the amino acid sequence QGYYNDGIWA (SEQ ID NO:20).
      2. The antibody of embodiment 1, wherein the V_{H} polypeptide comprises one, any combination of, or each of the mutations E2V, E5L/K, E15G/T, R44K, R85S/N, T87R/D, A89E/V, and P112Q.
      3. The antibody of embodiment 2, wherein the V_{H} polypeptide comprises each of the mutations E2V, E5L/K, E15G/T, R44K, R85S/N, T87R/D, A89E/V, P112Q.
      4. The antibody of embodiment 2 or embodiment 3, wherein the E5L/K mutation is E5L.
      5. The antibody of any one of embodiments 2 to 4, wherein the E15G/T mutation is E15G.
      6. The antibody of any one of embodiments 2 to 5, wherein the R85S/N mutation is R85S.
      7. The antibody of any one of embodiments 2 to 6, wherein the T87R/D mutation is T87R.
      8. The antibody of any one of embodiments 2 to 7, wherein the A89E/V mutation is A89E.
      9. The antibody of any one of embodiments 2 to 8, wherein the V_{H} polypeptide comprises one, any combination of, or each of the mutations Q1E, K13Q, T19R, T21S, T23A, T84N, T93V, and F95Y.
      10. The antibody of any one of embodiments 2 to 9, wherein the V_{H} polypeptide comprises a V_{H} CDR2 comprising the amino acid sequence CIYTSSGSAYYADSVKG (SEQ ID NO:16).
      11. The antibody of any one of embodiments 2 to 9, wherein the V_{H} polypeptide comprises one, any combination of, or each of the mutations T74D, S75N/T, S76_T77insK, T77N/S, V79L, and T80Y/V.
      12. The antibody of embodiment 11, wherein the T77N/S mutation is T77N.
      13. The antibody of embodiment 11 or embodiment 12, wherein the T80Y/V mutation is T80Y.
      14. The antibody of any one of embodiments 11 to 13, wherein the V_{H} polypeptide comprises a V_{H} CDR2 comprising the amino acid sequence CIYTSSGSAYYADSVKG (SEQ ID NO:16).
      15. The antibody of embodiment 2 or embodiment 3, wherein the V_{H} polypeptide comprises one, any combination of, or each of the mutations Q3T, E5L/K, G9P, G10V, E15G/T, G16E, S17T, A41P, G45A, T74D, S75N/T, S76_T77insK, T77N/S, T78Q, T80Y/V, Q82T, R85S/N, L86M, T87R/D, A88P, and A89E/V.
      16. The antibody of embodiment 15, wherein the E5L/K mutation is E5K.
      17. The antibody of embodiment 15 or embodiment 16, wherein the E15G/T mutation is E15T.
      18. The antibody of any one of embodiments 15 to 17, wherein the S75N/T mutation is S75T.
      19. The antibody of any one of embodiments 15 to 18, wherein the T77N/S mutation is T77S.
      20. The antibody of any one of embodiments 15 to 19, wherein the T80Y/V mutation is T80V.
      21. The antibody of any one of embodiments 15 to 20, wherein the R85S/N mutation is R85N.
      22. The antibody of any one of embodiments 15 to 21, wherein the T87R/D mutation is T87D.
      23. The antibody of any one of embodiments 15 to 22, wherein the A89E/V mutation is A89V.
      24. The antibody of any one of embodiments 1 to 3, wherein the V_{H} polypeptide comprises 80% or greater, 85% or greater, 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in one of SEQ ID Nos: 2-6.
      25. The antibody of any one of embodiments 2 to 9, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 3.
      26. The antibody of embodiment 10, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 5.
      27. The antibody of any one of embodiments 11 to 13, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 2.
      28. The antibody of embodiment 14, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 4.
      29. The antibody of any one of embodiments 15 to 23, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 6.
      30. The antibody of any one of embodiments 1 to 29, wherein the V_{L} polypeptide comprises one, any combination of, or each of the mutations T7S, P10T/S, V11L, A14S, G17D/E, T18R, P45A/V, N47K/R, K65S, Q72E/D, D79S, C82P, A85F/V, G103Q, E106K, V108E, and V109I.
      31. The antibody of embodiment 30, wherein the V_{L} polypeptide comprises each of the mutations T7S, P10T/S, V11L, A14S, G17D/E, T18R, P45A/V, N47K/R, K65S, Q72E/D, D79S, C82P, A85F/V, G103Q, E106K, V108E, and V109I.
      32. The antibody of embodiment 30 or embodiment 31, wherein the V_{L} polypeptide comprises one, any combination of, or each of the mutations G17D, S22T, Q44K, N47K, and E81Q.
      33. The antibody of any one of embodiments 30 to 32, wherein the P10T/S mutation is P10T.
      34. The antibody of any one of embodiments 30 to 33, wherein the P45A/V mutation is P45A.
      35. The antibody of any one of embodiments 30 to 34, wherein the Q72E/D mutation is Q72E.
      36. The antibody of any one of embodiments 30 to 35, wherein the A85F/V mutation is A85F.
      37. The antibody of any one of embodiments 33 to 36, wherein the V_{L} polypeptide comprises one, any combination of, or each of the mutations A1 D, Q2I, V3Q, and L4M.
      38. The antibody of any one of embodiments 30 to 32, wherein the V_{L} polypeptide comprises the mutation D83E.
      39. The antibody of any one of embodiments 30 to 32 or 38, wherein the P10T/S mutation is P10S.
      40. The antibody of any one of embodiments 30 to 32, 38, or 39, wherein the P45A/V mutation is P45V.
      41. The antibody of any one of embodiments 30 to 32 or 38 to 40, wherein the Q72E/D mutation is Q72D.
      42. The antibody of any one of embodiments 30 to 32 or 38 to 41, wherein the A85F/V mutation is A85V.
      43. The antibody of any one of embodiments 38 to 42, wherein the V_{L} polypeptide comprises one, any combination of, or each of the mutations A1 D, Q2I, V3Q, and L4M.
      44. The antibody of embodiment 30 or embodiment 31, wherein the V_{L} polypeptide comprises one, any combination of, or each of the mutations A1E, S9A, P10T, A13L, V15P, G17E, V19A, I21L, P45A, N47R, V60I, S62A, Q72D, D83E, A85F, T87V, and V107L.
      45. The antibody of embodiment 44, wherein the V_{L} polypeptide comprises each of the mutations A1E, S9A, P10T, A13L, V15P, G17E, V19A, I21L, P45A, N47R, V60I, S62A, Q72D, D83E, A85F, T87V, and V107L.
      46. The antibody of embodiment 1, 30 or 31, wherein the V_{L} polypeptide comprises 80% or greater, 85% or greater, 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in one of SEQ ID Nos: 8-12.
      47. The antibody of any one of embodiments 33 to 36, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 9.
      48. The antibody of embodiment 37, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 8.
      49. The antibody of any one of embodiments 38 to 42, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 11.
      50. The antibody of embodiment 43, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 10.
      51. The antibody of embodiment 44 or embodiment 45, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 12.
      52. An antibody that specifically binds to Vaccinia Virus B5 antigen (VV B5), wherein the antibody comprises:
a variable heavy chain (V_{H}) polypeptide comprising:

a V_{H} CDR 1 comprising the amino acid sequence
(SEQ ID NO: 13)
SSYYMC,
a V_{H} CDR2 comprising the amino acid sequence
(SEQ ID NO: 16)
CIYTSSGSAYYADSVKG,
and
a V_{H} CDR3 comprising the amino acid sequence
(SEQ ID NO: 17)
NAVGSSYYLYL;
and
a variable light chain (VL) polypeptide comprising:
a V_{L} CDR1 comprising the amino acid sequence
(SEQ ID NO: 18)
QASQSVAGNNYLS,
a V_{L} CDR2 comprising the amino acid sequence
(SEQ ID NO: 19)
SVSTLAS,
and
a V_{L} CDR3 comprising the amino acid sequence
(SEQ ID NO: 20)
QGYYNDGIWA.

53. The antibody of embodiment 52, wherein the antibody is a humanized antibody.
54. The antibody of any one of embodiments 1 to 53, wherein the antibody is an IgG.
55. The antibody of embodiment 54, wherein the antibody comprises a human Fc domain.
56. The antibody of any one of embodiments 1 to 53, wherein the antibody is selected from the group consisting of: a Fab, a F(ab′)₂, and a F(ab′).
57. The antibody of any one of embodiments 1 to 53, wherein the antibody is a single chain antibody.
58. The antibody of embodiment 57, wherein the single chain antibody is an scFv.
59. The antibody of any one of embodiments 1 to 58, wherein the antibody is a bispecific antibody comprising a first antigen-binding domain comprising a V_{H} polypeptide-V_{L} polypeptide pair as defined in any one of embodiments 1 to 53.
60. The antibody of embodiment 59, wherein the bispecific antibody comprises a second antigen-binding domain that specifically binds an antigen other than a Vaccinia Virus B5 antigen.
61. The antibody of embodiment 60, wherein the antigen other than a Vaccinia Virus B5 antigen is an immune cell surface antigen.
62. The antibody of embodiment 61, wherein the immune cell surface antigen is an immune effector cell surface antigen.
63. The antibody of embodiment 62, wherein the immune cell surface antigen is a T cell surface antigen.
64. The antibody of embodiment 63, wherein the antigen is a T cell stimulatory molecule.
65. The antibody of embodiment 64, wherein the T cell stimulatory molecule is CD3 or CD28.
66. The antibody of embodiment 62, wherein the immune cell surface antigen is a natural killer (NK) cell surface antigen.
67. The antibody of embodiment 62, wherein the immune cell surface antigen is a macrophage cell surface antigen.
68. A fusion protein, comprising: a chain of an antibody of any one of embodiments 1 to 53 fused to a heterologous sequence of amino acids.
69. The fusion protein of embodiment 68, wherein the heterologous sequence of amino acids is fused to the C-terminus of the chain of the antibody.
70. The fusion protein of embodiment 68 or embodiment 69, wherein the antibody is the single chain antibody of embodiment 57 or 58.
71. The fusion protein of embodiment 70, wherein the fusion protein is a chimeric antigen receptor (CAR) comprising:
the single chain antibody;
a transmembrane domain; and
an intracellular signaling domain.
   72. A conjugate, comprising:
an antibody of any one of embodiments 1 to 67 or a fusion protein of any one of embodiments 68 to 71; and
an agent conjugated to the antibody or fusion protein.
   73. The conjugate of embodiment 72, wherein the agent is selected from the group consisting of: a chemotherapeutic agent, a toxin, a radiation sensitizing agent, a radioactive isotope, a detectable label, and a half-life extending moiety.
   74. The conjugate of embodiment 73, wherein the radioactive isotope is a therapeutic radioactive isotope.
   75. The conjugate of embodiment 73, wherein the detectable label is a radiolabel.
   76. The conjugate of embodiment 75, wherein the radiolabel is Zirconium-89 (⁸⁹Zr).
   77. The conjugate of any one of embodiments 72 to 76, wherein the agent is conjugated to the antibody or fusion protein via a non-cleavable linker.
   78. The conjugate of any one of embodiments 72 to 76, wherein the agent is conjugated to the antibody or fusion protein via a cleavable linker.
   79. The conjugate of embodiment 78, wherein the cleavable linker is an enzyme-cleavable linker.
   80. The conjugate of embodiment 79, wherein the linker is cleavable by a lysosomal protease.
   81. The conjugate of embodiment 80, wherein the linker is cleavable by cathepsin or plasmin.
   82. A nucleic acid encoding a variable heavy chain (V_{H}) polypeptide, a variable light chain (V_{L}) polypeptide, or both, of an antibody of any one of embodiments 1 to 67.
   83. A nucleic acid encoding the fusion protein of any one of embodiments 68 to 71.
   84. An expression vector comprising the nucleic acid of embodiment 82 or embodiment 83.
   85. A cell comprising:
the nucleic acid of embodiment 82 or embodiment 83; or
the expression vector of embodiment 84.
   86. The cell of embodiment 85, wherein the cell comprises the nucleic acid of embodiment 83 or an expression vector comprising the nucleic acid of embodiment 83.
   87. The cell of embodiment 86, wherein the nucleic acid encodes the CAR of embodiment 71 and the cell expresses the CAR on its surface.
   88. The cell of embodiment 87, wherein the cell is an immune cell.
   89. The cell of embodiment 88, wherein the cell is an immune effector cell.
   90. The cell of embodiment 89, wherein the cell is a T cell.
   91. The cell of embodiment 89, wherein the cell is an NK cell.
   92. The cell of embodiment 89, wherein the cell is a macrophage.
   93. A cell comprising:
a first nucleic acid encoding the variable heavy chain (V_{H}) polypeptide of an antibody of any one of embodiments 1 to 53; and
a second nucleic acid encoding the variable light chain (V_{L}) polypeptide of the antibody.
   94. The cell of embodiment 93, comprising:
a first expression vector comprising the first nucleic acid; and
a second expression vector comprising the second nucleic acid.
   95. A pharmaceutical composition comprising the cell of any one of embodiments 85 to 94.
   96. The pharmaceutical composition of embodiment 95, comprising the cell of any one of embodiments 87 to 92.
   97. The pharmaceutical composition of embodiment 95 or embodiment 96, further comprising a pharmaceutically acceptable carrier.
   98. A method of producing the antibody of any one of embodiments 1 to 53, comprising culturing the cell of any one of embodiments 85 to 94 under conditions suitable for the cell to express the antibody, wherein the antibody is produced.
   99. A pharmaceutical composition, comprising:
the antibody of any one of embodiments 1 to 67; and
a pharmaceutically acceptable carrier.
   100. A pharmaceutical composition, comprising:
the fusion protein of any one of embodiments 68 to 71; and
a pharmaceutically acceptable carrier.
   101. A pharmaceutical composition, comprising:
the conjugate of any one of embodiments 72 to 81; and
a pharmaceutically acceptable carrier.
   102. A kit, comprising:
the pharmaceutical composition of any one of embodiments 95 to 97 or 99 to 101; and
instructions for administering the pharmaceutical composition to an individual in need thereof.
   103. The kit of embodiment 102, wherein the pharmaceutical composition is present in one or more unit dosages.
   104. The kit of embodiment 102, wherein the pharmaceutical composition is present in two or more unit dosages.
   105. A method, comprising:
administering the pharmaceutical composition of any one of embodiments 95 to 97 or 99 to 101 to an individual having cancer, wherein the individual comprises cancer cells infected with Vaccinia Virus (VV), and wherein the antibody, fusion protein or conjugate is targeted to the infected cancer cells by VV B5 antigens expressed on the surface of the infected cancer cells.
   106. The method according to embodiment 105, further comprising, prior to administering the pharmaceutical composition to the individual, infecting the cancer cells by administering VV to the individual.
   107. The method according to embodiment 105 or 106, wherein the method is a method of treating the cancer of the individual.
   108. The method according to any one of embodiments 105 to 107, wherein the pharmaceutical composition of embodiment 101 is administered to the individual, wherein the conjugate comprises the antibody conjugated to a detectable label or radioactive isotope which is an in vivo imaging agent, and wherein the method comprises imaging the infected cancer cells in the individual using the in vivo imaging agent.

The following examples are offered by way of illustration and not by way of limitation.

### EXPERIMENTAL

### Example 1—Design and Expression of Humanized V-Region Sequences

This example describes the design and expression of humanized variable regions of the rabbit anti-VV B5 antibody A048. The parental A048 antibody is described in International Patent Application No. PCT/CA202/051230. The A048 variable regions (V-regions) were humanized using sequence based-CDR grafting. An in silico homology modeling tool (Schrodinger Bioluminate) was used to analyze best fitting candidate templates from closest aligning human germline sequences for each variable (V) domain, with rabbit CDRs grafted in and compared to the non-humanized A048 parental rabbit antibody and germline antibody. The complementarity determining region (CDR) and framework (FW) sequences were reviewed, taking into consideration a combination of Kabat, IMGT, Chothia, and in-house CDR annotations to define involvement in antigen interaction. The start of FW1 was selected based on IMGT database rabbit antibody sequences. Structural analysis of the homology model assessed conformational or sequence deviations which may impact CDR stability and conformation, and suitable back mutations were identified.

IGHV3_23 was found to be the best fit for sequence homology for the A048 variable heavy chain (VH) domain. The common germline IGHJ4_1 was found to be the best fit for A048 VH FW4 due to high sequence homology. IGKV1_5 was found to be the best fit for VL domain sequence homology. Additionally, IGKV1_27, which differs by five amino acids, was selected, as some amino acid positions (IGKV1_5 to IGKV1_27; T10S, A45V, E72D, D83E, F85V) may impact CDR stability based on in silico modelling. IGKJ4_1 was chosen for VL FW4 due to high sequence homology. In 2 VH candidates (A048_H3 and A048_H4), 3 amino acids were mutated to the human sequence N63D, W64S, A65V rabbit to human) in the extended end of CDR2, thereby making the CDR2 more human. This region is relatively distant from VHCDR3, so likely not involved in antigen binding but solvent exposed with a potential impact on immunogenicity. In 2 VH candidates (A048_H2 and A048_H4), back mutation from human to rabbit in FW3 (also known as the HV4 loop) were introduced (human to rabbit: D74T, N75S, S76_T77delK, N77T, L79V, Y80T) to assess potential impact on binding. In 2 VL candidates (A048_L2 and A048_L4) n-termini exchange in FW1 from human to rabbit were introduced (D1A, 12Q, Q3V, M4L) to assess potential impact on binding.

Humanized VH domains (A048_H1, A048_H2, A048_H3, A048_H4) and humanized VL domains (A048_L1, A048_L2, A048_L3, A048_L4) were combined to generate 16 humanized IgGs for assessment.

One additional humanized antibody was generated using structural homology modelling first to identify the best template based on composite score and resolution using Schrödinger Bioluminate software. Structural analysis of the CDR grafted prototype V-domains checked for conformational and sequence deviations. The human Fv #3L7F template was selected for structural grafting of rabbit CDRs of A048. The best matching human germlines for 3L7F were IGHV2 for VH, while no dominant IGKV family was identified. No conformational or sequence deviations which could potentially impact CDR stability and conformation were observed in silico. Therefore, no back mutations were introduced once the A048 rabbit CDRs were grafted into #3L7F VH and VL as described by A048_H5L5.

The sequences of the parental and humanized VH and VL domains are shown in Table 1.

The humanized VH and VL sequences were synthesized and cloned into the ptt5 expression vector (NRC) containing either the constant region of human IgG1 heavy chain or human kappa light chain. To produce recombinant antibody, VH and VL chain plasmids were transfected into HEK293 cells using FectoPro (Polyplus, Cat #116-001). After 120 hours of secretion, the antibody-containing supernatant was cleared of cells by centrifugation and sterile filtration (0.22 μm). Antibodies were purified using Protein A (Purolite Praesto Jetted A050) resin. Concentrations and yields of the purified antibodies were determined by UV absorbance measurements at 280 nm (A280) using a biospectrophotometer. Concentration values were calculated using A280 extinction coefficients determined for the individual mAb sequences. Yields (mg/L conditioned media, ‘CM’) determined by the A280 measurements are shown in Table 9. Yields of the humanized antibodies, which were 4- to 8-fold higher than the yield of the parental chimeric antibody, suggest good manufacturability of the humanized antibodies. The purity of the antibodies was tested by SDS-PAGE. The percent intact antibody was determined by HPLC-SEC. Antibody (12 μl) was added to a glass vial insert in a standard HPLC vial. Sample (10 μl) was injected into HPLC (Agilent) with a WCL006 SEC column (Wyatt Technology, Cat #: WTC-010S5) and eluted with PBS, pH 7.4 for 40 min at a flow rate of 0.5 mL/min. The percentage of intact antibody was determined by the peak area of the mAb peak among the peak area of other peaks if present. HPLC-SEC results are shown in Table 9.

### Example 2—Humanized Antibody Binding to VV B5

Humanized antibodies were assessed for binding to VV B5 protein by ELISA. B5 protein was generated using the B5 sequence from the Wyeth Vaccinia strain (Dr. John Bell OHRI). The B5 gene was synthesised with a C-terminal AVI tag for biotinylation and a HIS₆ tag for purification, in ptt5 expression vector (NRC) and transfected into HEK293-6E cells (NRC) using 293fectin (Thermo, Cat #12347019). After 96 hours of secretion, the protein-containing supernatant was collected and purified by IMAC and SEC using AKTA Pure FPLC system. The protein was further purified by SEC using a Superdex 200 Increase 10/300 GL column (Cytiva, Cat #: 28990944) connected with a Superdex 75 Increase 10/300 GL column (Cytiva, Cat #: 29148721) with PBS, pH 7.4. The fractions containing monomeric protein were combined and concentrated through an Amicon Ultra centrifugal filter device, 10 k MWCO.

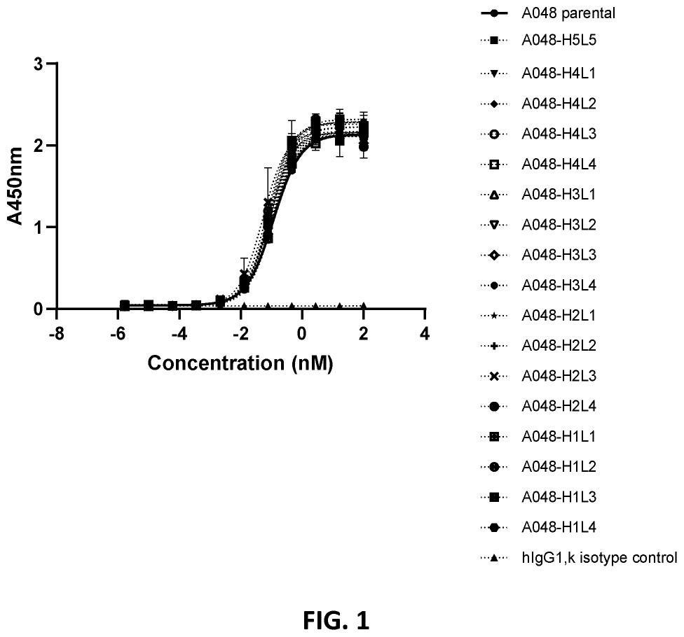

For binding ELISA, B5 monomer was coated at 1 μg/mL on a 96-well ELISA plate (Greiner Bio-One High Bind) in PBS, and stored at 4° C. overnight. Following washing in PBS/Tween wash buffer, the plate was incubated in blocking buffer (1% BSA/PBS) for 1 h at RT. Blocking buffer was removed and each anti-B5 hIgG1 antibody was added in a serial titration from 100 nm in blocking buffer. After 1 h incubation at RT, the plate was washed three times and secondary antibody (rabbit anti-hIgG HRP) was added for 1 h. The plate was washed and TMB added, followed by addition of 2M H₂SO₄ stopping solution. OD450 was read on a spectrophotometer. All humanized antibodies bound to B5 protein at a similar EC50 as the A048 rabbit parental antibody as shown in Table 6 below and FIG. 1.

TABLE 6
Binding of Humanized Antibodies to VV B5

| Antibody | EC50 pM |
|---|---|
| A048 parental | 115.6 |
| A048-H5L5 | 104 |
| A048-H4L1 | 95.27 |
| A048-H4L2 | 106.8 |
| A048-H4L3 | 114.3 |
| A048-H4L4 | 114 |
| A048-H3L1 | 85.43 |
| A048-H3L2 | 83.47 |
| A048-H3L3 | 97.91 |
| A048-H3L4 | 106.2 |
| A048-H2L1 | 96.95 |
| A048-H2L2 | 91.38 |
| A048-H2L3 | 51.49 |
| A048-H2L4 | 84.44 |
| A048-H1L1 | 86.17 |
| A048-H1L2 | 71.71 |
| A048-H1L3 | 80.18 |
| A048-H1L4 | 101.6 |

### Example 3—Humanized Antibody Binding to Vaccinia Virus-Infected Cells

Binding to Vaccinia Virus-infected cancer cells was determined by flow cytometry. HT29 (ATCC HTB-38) and SKOV3 (ATCC #HTB-77) cells were seeded at 27.3×10⁶ cells/well in T175 tissue culture flasks (Corning, Cat #353112) in McCoy's 5A (Gibco, Cat #16600-082)+10% fetal bovine serum (Corning, Cat #35-015-CV) and left to adhere overnight at 37° C., 5% CO2. The next day, culture medium was aspirated and cells were infected with either VVdd(eGFP) or VVcopenhagen(YFP) (Vaccinia virus strains Western Reserve and Copenhagen respectively, both gifts from Dr. John Bell, OHRI) at a multiplicity of infection (MOI) of 0.1, or mock-infected, in 27.3 mL serum-free McCoy's 5A (1×10⁴ PFU/mL) at 37° C., 5% CO₂. Infection medium was replaced at 2 h post-infection with 27.3 mL McCoy's 5A+10% fetal bovine serum and incubated at 37° C., 5% CO₂. At 48 h post-infection, culture medium from each flask was aspirated into a separate 50 mL falcon tube. Cells were rinsed with 10 mL phosphate buffered saline (Gibco, Cat #10010-023) and the wash added to the corresponding tube. Cells were dissociated by incubation in 10 mL of cell dissociation buffer (Gibco, Cat #13151014)/flask at 37° C. for 60 min. Meanwhile, cells in the culture supernatant were pelleted by centrifuging tubes at 400×g for 5 min and discarding the supernatant. After dissociation, cells were resuspended in 10 mL PBS and transferred to the corresponding tube before pelleting by centrifugation at 400×g for 5 minutes. Supernatants were discarded and cells stained for viability using LIVE/DEAD Fixable Violet Dead Cell Stain diluted 1:1000 in 27.3 mL PBS (Invitrogen, Cat #L34955) for 30 min at 4° C. After staining, cells were pelleted by centrifugation at 400×g for 5 minutes and resuspended in 27.3 mL staining buffer (phosphate buffered saline+2 mM EDTA+0.5% bovine serum albumin). Cells were plated at 50 μl/well (5×10⁴ cells/well) in 96-well V-bottom plates (Corning, Cat #3894) and centrifuged at 400×g for 5 min. Cells were resuspended in 25 μl/well human anti-E5 antibody (A048-H1 L1, A048-H1 L2, A048-H1 L3, A048-H1 L4, A048-H2L1, A048-H2L2, A048-H2L3, A048-H2L4, A048-H3L1, A048-H3L2, A048-H3L3, A048-H3L4, A048-H4L1, A048-H4L2, A048-H4L3, A048-H4L4, A048-H5L5, A048, or human IgG1k isotype control) at a concentration of 0.0034, 0.017, 0.085, 0.43, 2.1, 10.7, 53.6, or 268 nM and incubated at 4° C. for 1 h before addition of 100 μl staining buffer and centrifugation at 400×g for 5 min.

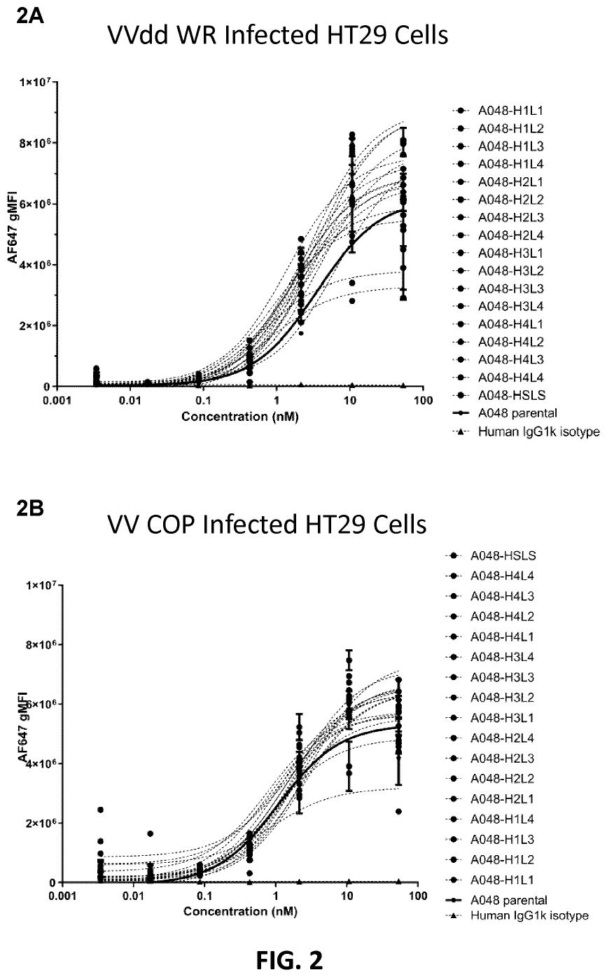

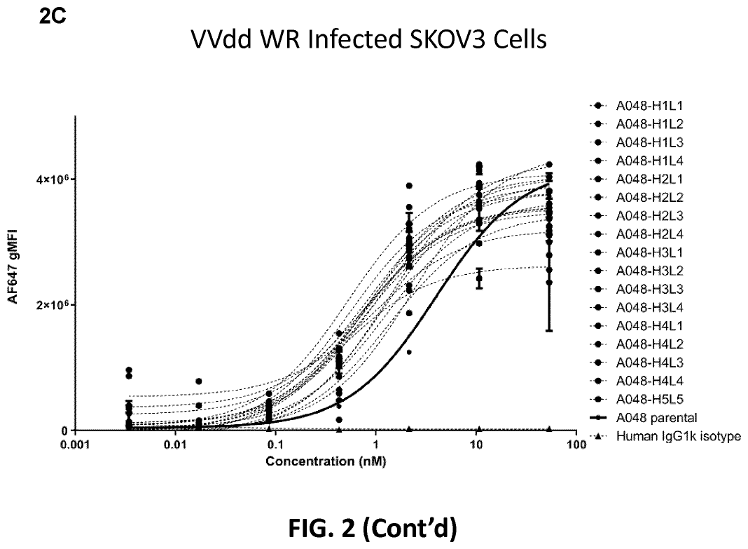

Primary antibody binding was detected by incubating samples with 25 μl of 2 μg/mL AlexaFluor-647-conjugated goat anti-human IgG (Jackson ImmunoResearch, Cat #109-605-098) in staining buffer at 4° C. for 30 min. Cells were then fixed in 50 μl/well 4% paraformaldehyde in PBS (Thermo Scientific, Cat #19943-K2) for 15 min at room temperature before washing with 100 μl staining buffer. Samples were resuspended in 50 μl staining buffer for data acquisition on an Intellicyte iQue Screener PLUS flow cytometer and analysis using Intellicyt ForeCyt software. All 17 humanized antibodies and A048 parental anti-E5 antibody showed similar binding to Vaccinia virus-infected cancer cells as shown in Table 7 below and FIG. 2A-2C. Shown in FIGS. 2A, 2B and 2C is humanized antibody binding to HT29 cells infected with Western Reserve VV strain, HT29 cells infected with Copenhagen VV strain, and SKOV3 cells infected with Western Reserve VV strain, respectively.

TABLE 7
Binding of Humanized Antibodies to VV-Infected Cells

| | EC50 nM | | |
|---|---|---|---|
| | HT29 | SKOV3 | HT29 |
| Antibody | WR | WR | Cop |
| A048-H1L1 | 0.97 | 1.03 | 2.37 |
| A048-H1L2 | 2.20 | 0.76 | 0.77 |
| A048-H1L3 | 3.48 | 1.20 | 1.88 |
| A048-H1L4 | 0.68 | 0.44 | 0.98 |
| A048-H2L1 | 1.37 | 0.53 | 1.48 |
| A048-H2L2 | 1.71 | 0.80 | 1.28 |
| A048-H2L3 | 1.73 | 0.55 | 0.77 |
| A048-H2L4 | 1.68 | 0.69 | 2.85 |
| A048-H3L1 | 0.86 | 0.94 | 0.89 |
| A048-H3L2 | 1.32 | 0.67 | 1.87 |
| A048-H3L3 | 1.81 | 0.83 | 1.03 |
| A048-H3L4 | 3.78 | 1.28 | 1.96 |
| A048-H4L1 | 2.77 | 0.88 | 1.34 |
| A048-H4L2 | 6.59 | 0.71 | 1.78 |
| A048-H4L3 | 3.50 | 1.37 | 1.12 |
| A048-H4L4 | 3.05 | 2.36 | 1.32 |
| A048-H5L5 | 2.28 | 1.36 | 2.39 |
| A048 parental | 3.51 | 3.98 | 1.08 |
| Human IgG1k isotype | 0.02 | 0.01 | 0.01 |

### Example 4—Humanized Antibody Affinity to VV B5

Affinity of the humanized antibodies was assessed using label-free biolayer interferometry on the OctetRed96E® biolayer interferometer according to standard protocols. Humanized anti-B5 antibodies were captured using anti-human capture (AHC) biosensors (Sartorius) with loading for 600 s. Loaded sensors were then dipped into B5 monomer analyte (40 nM, titrated 1:2 down), with 600 s for association and 1800 s dissociation. Sensors were regenerated for 30 s. A control biosensor without antibody capture was used for double reference subtraction. Data was analyzed using Octet Data Analysis software v11.1. The parental A048 antibody has a KD of 9.5 nM, and all 17 humanised antibodies were similar, ranging from 3-12 nM KD (Table 8).

TABLE 8
Binding of Humanized Antibodies to VV-Infected Cells

| Antibody | KD nM | Ka (1/Ms) | Kd (1/s) |
|---|---|---|---|
| A048-H1L1 | 9.96 | 7.66 × 10⁴ | 7.63 × 10⁻⁴ |
| A048-H1L2 | 9.71 | 7.86 × 10⁴ | 7.637 × 10⁻⁴ |
| A048-H1L3 | 8.76 | 9.64 × 10⁴ | 8.457 × 10⁻⁴ |
| A048-H1L4 | 12.0 | 7.94 × 10⁴ | 9.527 × 10⁻⁴ |
| A048-H2L1 | 7.21 | 9.30 × 10⁴ | 6.717 × 10⁻⁴ |
| A048-H2L2 | 7.91 | 8.81 × 10⁴ | 6.977 × 10⁻⁴ |
| A048-H2L3 | 9.23 | 8.21 × 10⁴ | 7.587 × 10⁻⁴ |
| A048-H2L4 | 6.23 | 8.16 × 10⁴ | 5.117 × 10⁻⁴ |
| A048-H3L1 | 4.37 | 1.07 × 10⁵ | 4.677 × 10⁻⁴ |
| A048-H3L2 | 3.83 | 1.03 × 10⁵ | 3.967 × 10⁻⁴ |
| A048-H3L3 | 7.71 | 9.49 × 10⁴ | 7.327 × 10⁻⁴ |
| A048-H3L4 | 8.57 | 8.41 × 10⁴ | 7.217 × 10⁻⁴ |
| A048-H4L1 | 6.72 | 7.10 × 10⁴ | 4.777 × 10⁻⁴ |
| A048-H4L2 | 7.69 | 6.24 × 10⁴ | 4.807 × 10⁻⁴ |
| A048-H4L3 | 6.31 | 7.52 × 10⁴ | 4.757 × 10⁻⁴ |
| A048-H4L4 | 9.60 | 5.70 × 10⁴ | 5.487 × 10⁻⁴ |
| A048-H5L5 | 8.55 | 6.08 × 10⁴ | 5.207 × 10⁻⁴ |
| A048 | 9.51 | 6.24 × 10⁴ | 6.007 × 10⁻⁴ |
| (parental) | | | |

### Example 5—Humanized Antibody Self-Association Determination

Propensities of self-association of antibodies were determined from affinity-capture self-interaction nanoparticle spectroscopy (AC-SINS) using gold nanoparticles (Au—NP) (Ted Pella, Cat #: 15705) (1,2). Briefly, goat IgG (Jackson, Cat #: 005-000-003) and goat anti-human Fc IgG (Jackson, Cat #: 109-005-098, 1:4 mole ratio) were used to coat the Au—NP. The mixture was incubated at room temperature for 1 hour with rotation. Final concentration of poly(ethylene glycol) methyl ether thiol was added to quench the reaction. The conjugated Au—NP was then concentrated by centrifugation at 13,000×g for 5 min. 1/20 part of original volume of PBS was used to resuspend the Au—NP. 100 μL of 50 μg/mL of each antibody in quadruplicate was mixed with 10 μL of concentrated Au—NP on 96-well plate (Thermo Scientific™ Nunc™ 96-Well Polypropylene MicroWell™ Plates (Cat #: 12565369)). The mixture was incubated at room temperature for 2 hrs. The wavelength scan was measured with Synergy Neo2 plate reader. The difference of maximum absorbance (Δλₘₐₓ) was calculated by subtracting of λₘₐₓ of each reaction with the one of PBS buffer. The data was analyzed with Linest function in Excel using second-order polynomial fitting. Data is shown in Table 9. Cetuximab (DIN:02271249, Lily), Trastuzumab (DIN:02240692, Roche) and Infliximab (DIN:02419475, Hospira) were used as controls. Infliximab has previously been reported as having a high AC-SINs (1), and based on the literature <11 is used as a cutoff score.

### Example 6—Humanized Antibody Polyreactivity Determination

Polyreactivity of the humanized antibodies against negatively charged biomolecules was determined by ELISA (3). Briefly, an ELISA plate (Nunc MaxiSorp™ flat-bottom, Thermo, Cat #: 44-2404-21) was coated with 5 μg/mL of human insulin (SigmaAlrich, Cat #: 19278) and 10 μg/mL of double stranded DNA (SigmaAlrich, Cat #: D1626-250MG) overnight. The plate was blocked with ELISA buffer (PBS, 1 mM EDTA, 0.05% Tween-20, pH 7.4). 10 μg/mL of test antibodies were loaded onto the plates in quadruplicates and incubated for 2 hrs. 0.01 μg/mL of goat anti-human Fc conjugated with HRP was then added and the plate was incubated for 1 hr. The signal was developed with TMB (Sigma, Cat #: T0440-1 L) and A₄₅₀ absorbance was measured with Synergy Neo2 plate reader. The signal was normalized with the signal with non-coated well for each antibody tested. Data is shown in Table 9. Cetuximab (DIN:02271249, Lily), Trastuzumab (DIN:02240692, Roche) and Infliximab (DIN:02419475, Hospira) were used as controls. These control antibodies have previously been reported as having low polyreactivity and the humanized anti-VV B5 antibodies are comparable.

### Example 7—Humanized Antibody Denaturing Temperatures

Denaturing temperatures (Tₘ) of the humanized antibodies were determined by differential scanning fluorimetry (DSF) using Protein Thermo Shift Dye Kit™ (ThermoFisher, Cat #: 4461146). Briefly, 31 μg/mL of antibody was used in each reaction. Melting curves of the antibodies were generated using an Applied Biosystems QuantStudio 7 Flex Real-Time PCR System with the recommended settings stated in the kit manual. The Tₘ of each antibody was then determined using the ThermoFisher Protein Thermal Shift software (v.1.3). Cetuximab (DIN:02271249, Lily), Trastuzumab (DIN:02240692, Roche) and Infliximab (DIN:02419475, Hospira) were used as controls. Based on the literature, a Tₘ1 of ≥65° C. is used as an acceptable cutoff. DSF, as well as HPLC-SEC, AC-SINS and polyreactivity data for the humanized antibodies is provided in Table 9.

TABLE 9
Humanized Antibody Yield and Biophysical Characteristics

| | A280 | | | | | |
|---|---|---|---|---|---|---|
| | Yield | DSF | | | Polyreactivity | |
| | (mg/ | Tm1 | HPLC-SEC | AC-SINS | Insulin | dsDNA |
| Antibody | LCM) | (° C.) | % intact | Δλ_{Max} | Score | Score |
| A048-H1L1 | 158.5 | 70.5 | 99.0 | 4.3 | 2.1 | 1.6 |
| A048-H1L2 | 141.5 | 70.7 | 94.4 | 3.7 | 2.5 | 1.7 |
| A048-H1L3 | 227.5 | 70.6 | 100.0 | 3.5 | 2.1 | 1.3 |
| A048-H1L4 | 143.5 | 70.4 | 100.0 | 3.3 | 2.1 | 1.4 |
| A048-H2L1 | 142 | 70.5 | 100.0 | 5.7 | 2.2 | 1.4 |
| A048-H2L2 | 144.5 | 71.2 | 100.0 | 4.5 | 2.5 | 1.7 |
| A048-H2L3 | 144.5 | 70.5 | 100.0 | 4.4 | 2.3 | 1.5 |
| A048-H2L4 | 125 | 70.6 | 100.0 | 4.4 | 2.4 | 1.5 |
| A048-H3L1 | 138 | 70.7 | 100.0 | 3.3 | 1.5 | 1.3 |
| A048-H3L2 | 127 | 70.7 | 100.0 | 2.6 | 1.6 | 1.3 |
| A048-H3L3 | 152.5 | 70.6 | 100.0 | 2.7 | 1.6 | 1.2 |
| A048-H3L4 | 141.5 | 70.6 | 100.0 | 2.5 | 1.5 | 1.2 |
| A048-H4L1 | 145.5 | 70.6 | 100.0 | 4.5 | 1.6 | 1.3 |
| A048-H4L2 | 134.5 | 70.6 | 97.1 | 3.7 | 1.8 | 1.3 |
| A048-H4L3 | 165.5 | 70.7 | 100.0 | 3.8 | 1.7 | 1.3 |
| A048-H4L4 | 144.5 | 70.6 | 100.0 | 3.1 | 1.7 | 1.3 |
| A048-H5L5 | 104.5 | 70.6 | 100.0 | 2.0 | 1.8 | 1.6 |
| A048 parental | 26 | 70.5 | 93.4 | 1.6 | 1.1 | 1.1 |
| Cetuximab | nd* | 71.1 | nd* | 4.4 | 2.5 | 1.4 |
| Trastuzumab | nd* | 70.6 | 100.0 | 4.6 | 1.8 | 1.9 |
| Infliximab | nd* | 71.9 | nd* | 24.3 | 2.9 | 2.0 |

### Example 8—Binding of Humanized Antibodies to Vaccinia Virus-Infected Cells Over Time

Described in this example is the determination of binding of example humanized anti-B5 antibodies of the present disclosure to vaccinia virus-infected cells over time.

HT29, SKOV3, and OVCAR3 cells were seeded at 20,000 cells/well in clear flat-bottomed 96-well tissue culture plates (Corning, Cat #353072) in complete culture medium. For HT29 and SKOV3 cells, complete culture medium was McCoy's 5A (Gibco, Cat #16600-082)+10% fetal bovine serum (Corning, Cat #35-015-CV). For OVCAR3 cells, complete culture medium was RPMI-1640 (Gibco, Cat #A10491-01)+0.01 mg/mL bovine insulin (Sigma Cat #10516-5ML)+20% fetal bovine serum and left to adhere overnight at 37° C., 5% CO₂. The next day, culture medium was aspirated and cells were infected with either VVddeGFP Western Reserve (‘WR’) or VVCopenhagen (YFP) virus (‘Cop’) at a multiplicity of infection (MOI) of 0.1 or 1, or mock-infected, in 100 μL serum-free McCoy's 5A at 37° C., 5% CO₂.

Infection medium was replaced at 2 h post-infection with 100 μL complete culture medium as indicated above and incubated at 37° C., 5% CO₂. At 2, 24, 48, 72, and 168 h post-infection, culture supernatants were transferred to V-bottom 96-well plates (Corning Cat #3894). Cells were then washed with 50 μL phosphate buffered saline (Gibco Cat #L34955) and the wash pooled with the supernatant. Cells were then dissociated by incubating them in 25 μL cell dissociation buffer (Gibco, Cat #13151014) per well at 37° C. for 30 min. Meanwhile, cells in the culture supernatant were pelleted by centrifuging the V-bottom plates at 400×g for 5 min and discarding the supernatant. After dissociation, cells were resuspended in 100 μL/well PBS and pooled with the harvested supernatant in V-bottom plates. Cells were centrifugated at 400×g for 5 minutes and resuspended in 50 μL/well LIVE/DEAD Fixable Violet Dead Cell Stain diluted 1:1000 in PBS (Invitrogen, Cat #L34955) for 30 min at 4° C. After staining, cells were washed in 100 μL/well staining buffer (phosphate buffered saline+2 mM EDTA+0.5% bovine serum albumin) and pelleted by centrifugation at 400×g for 5 minutes. Cells were resuspended in 50 μL/well AlexaFluor-647-conjugated humanized anti-vaccinia virus B5 antibody (A048-H1L4 or A048-H3L2) at a concentration of 10 nM and incubated at 4° C. for 1 hour before addition of 100 μL staining buffer and centrifugation at 400×g for 5 min. Cells were then fixed in 50 μL/well 4% paraformaldehyde in PBS (Thermo Scientific, Cat #19943-K2) for 15 min at room temperature before washing with 100 μL staining buffer. Samples were resuspended in 50 μL staining buffer for data acquisition on a Beckton Dickson LSRFortessa X-20 and analysis using FlowJo 10.8.1 software.

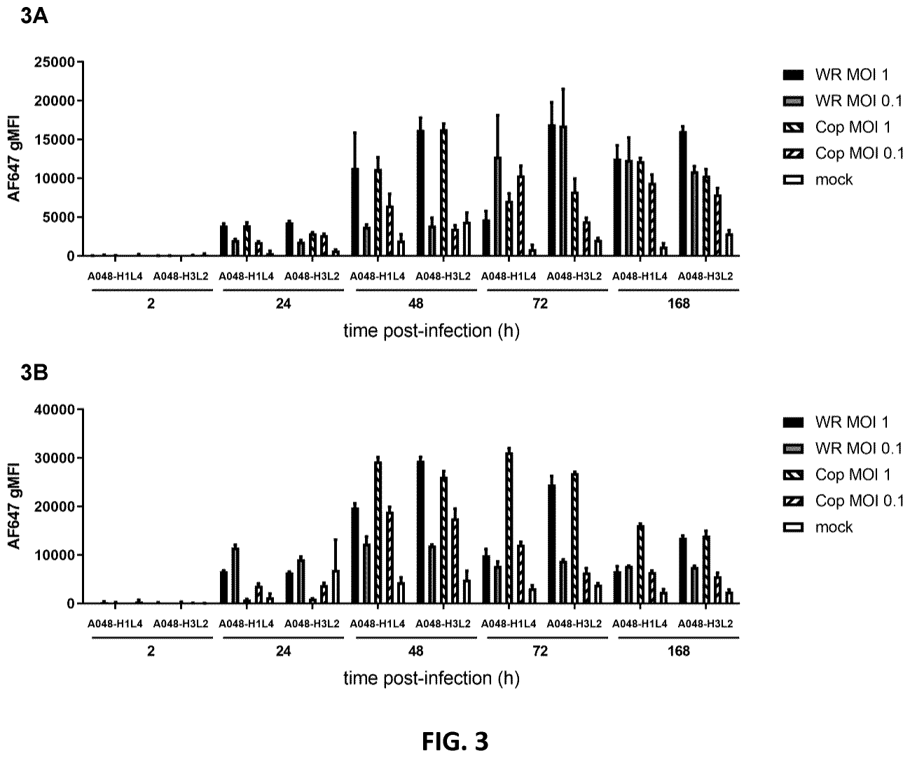

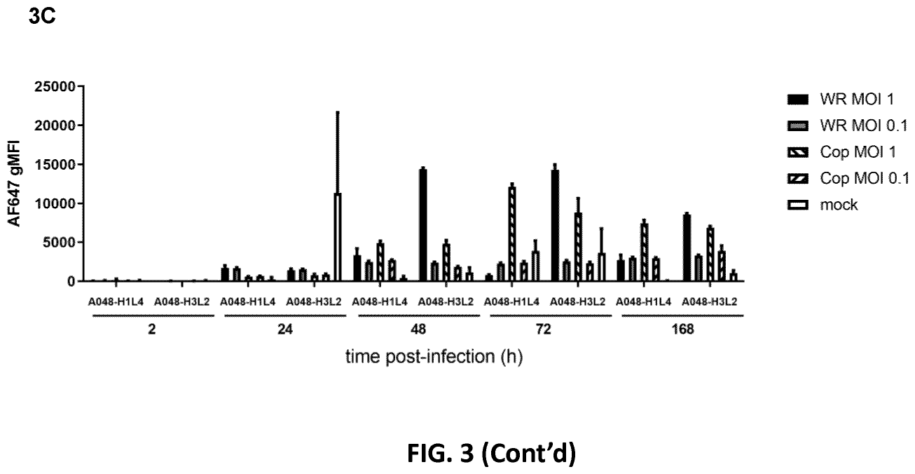

Results show that both A048-H1L4 and A048-H3L2 bind similarly to Vaccinia virus-infected cells (FIG. 3A—HT29 cells; FIG. 3B—SKOV3 cells; FIG. 3C—OVCAR3 cells). The data demonstrate that both antibodies bind multiple Vaccinia virus-infected cell lines from different cancer indications, and cells infected with different Vaccinia virus strains. Additionally, the data show that A048-H1L4 and A048-H3L2 binding to Vaccinia virus-infected cells can occur over an extended period of time.

### Example 9—Humanized Anti-VV B5 Chimeric Antigen Receptor (CAR) Design, Detection, and Activation Following Lentiviral Transduction of Human T Cells

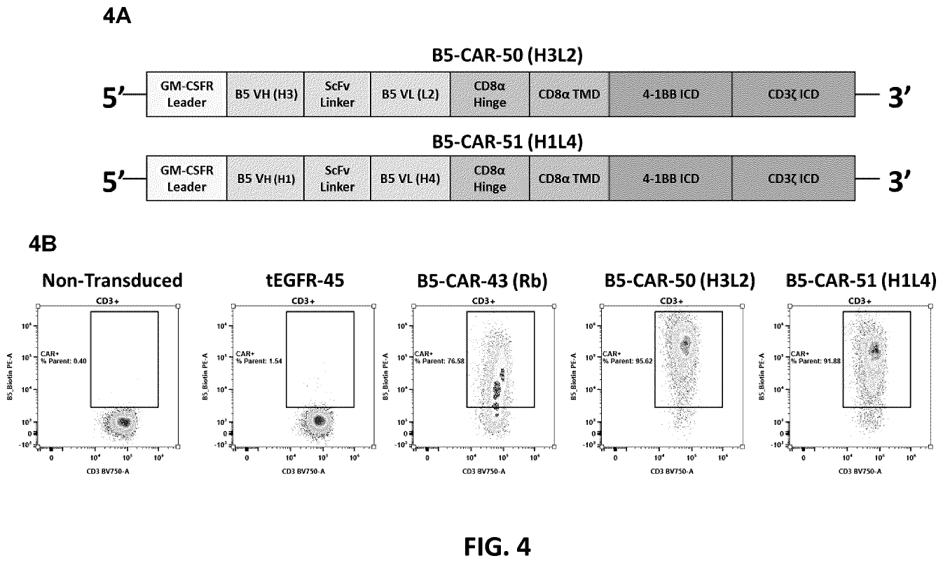

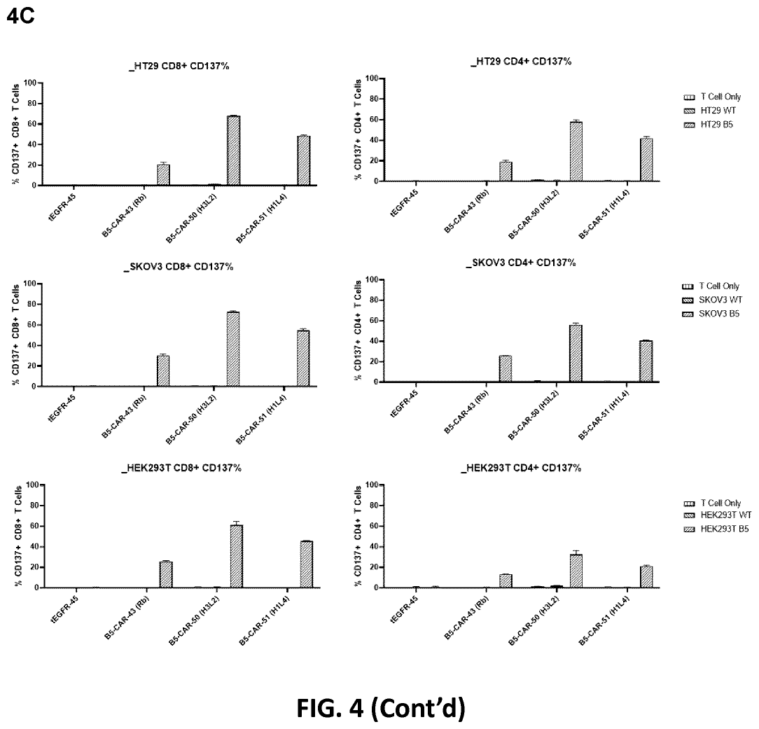

Described in this example are chimeric antigen receptors (CARs) comprising the humanized anti-VV B5 antibodies described elsewhere herein. In this particular example, and as schematically illustrated in FIG. 4A, the humanized anti-VV B5 CAR constructs comprise: a GM-CSFR a leader sequence; the humanized B5 scFv (in this example, A048_H3 and A048_L2, referred to as B5-CAR-050 or A048_Hi and A048_L4, referred to as B5-CAR-051); a human CD8α hinge and transmembrane domain; a human 4-1BB intracellular signaling domain; and a human CD3(intracellular signaling domain. Some CAR constructs may also include a reporter protein (e.g., eGFP or truncated EGFR (tEGFR)) separated by a ribosomal skip sequence, e.g., a T2A ribosomal skip sequence. The amino acid sequences of the CARs are provided in Table 5.

Gene fragments encoding B5-CAR-050 or B5-CAR-051 were synthesized by Twist Biosciences. Gene fragments were cloned into a 2^{nd} generation transfer plasmid (Twist Biosciences). Lentivirus encoding VV-CAR constructs are generated using the Lipofectamine™ 3000 transfection reagent protocol (ThermoFisher Scientific, Cat #L300015) and 2^{nd} generation packaging vectors (AddGene). To optimize CAR transduction, CD4⁺ and CD8⁺ T cell populations were isolated from healthy donor PBMC samples. 1×10⁶ healthy donor T cells were activated with Miltenyi TransAct™ (Miltenyi Biotec, Cat #130-111-160) and grown in Miltenyi TexMACS GMP (Miltenyi Biotec, Cat #170-076-309) media supplemented with 3% human serum (Sigma, Cat #H4522), gentamicin sulfate (Sandoz, DIN:02268531) and human interleukin-7 (Miltenyi Biotec, Cat #130-095-367) and interleukin-15 (Miltenyi Biotec, Cat #130-095-764) (10 ng/ml). 24 h after activation, T cells were transduced with B5-CAR-050 or B5-CAR-051 lentivirus at an MOI of 1.0-5.0. Cells were expanded for an additional 12 days at a density less than 1×10⁶ cells per ml. Shown in FIG. 4B is CD3+ and CAR expression data detected by a soluble B5 protein bound to Streptavidin-PE. In order, CAR expression is not present on non-transduced T cells and tEGFR (SEQ ID NO:54) vector-only transduced T cells. Anti-VV B5 CAR is detected on T cells transduced with a lentivirus encoding the parental rabbit B5-CAR-043 CAR construct. However, CAR expression is greatest in the two humanized CAR constructs (B5-CAR-050, B5-CAR-051).

Stable cell lines (in this example, cancer cell lines) expressing VV B5 antigen (e.g., B5 antigen from the Wyeth VV strain) were generated to enable CAR characterization. B5-CAR-050 or B5-CAR-051 CAR T cells were plated in a co-culture assay with either HT-29-B5 and HT-29-WT (ATCC HTB-38) cells, SKOV3-B5 and SKOV3-WT (ATCC HTB-77) cells, or HEK293T-B5 and HEK293T-WT (ATCC CRL-3216). Cells were cultured overnight (16 h) at a 1:1 Effector:Target (E:T) ratio (2×10⁵ total cells). The following day cells were washed with PBS, blocked with Human Trustain FcX (Biolegend, Cat #422302), and stained for CD8 PerCP (Biolegend, Cat #344708), CD3 BV750 (Biolegend, Cat #344846), CD137 APC (BD, Cat #561702), and CD4 AF700 (Biolegend, Cat #344622). Data is shown in FIG. 4C. B5-CAR-050 and B5-CAR-051 CAR T cells exhibited increased expression of CD137 when co-cultured with cells expressing the appropriate target antigen but minimal expression of CD137 when co-cultured with WT cells, indicative of target-specific activation. Both B5-CAR-050 and B5-CAR-051 CAR T cells exhibited heightened expression of CD137 as compared to parental B5-CAR-043 CAR T cells. This analysis shows that primary PBMC-derived human T cells can be readily transduced to express VV-CAR constructs and expanded post-transduction. Further, these data demonstrate that effector T cells transduced to express B5-CAR-050 or B5-CAR-051 CARs undergo activation upon encountering target cells bearing the VV B5 antigen.

### Example 10—Human T Cells Expressing B5-CAR-050 or B5-CAR-051 Exhibit Cytotoxicity Against VV B5-Expressing Target Cells

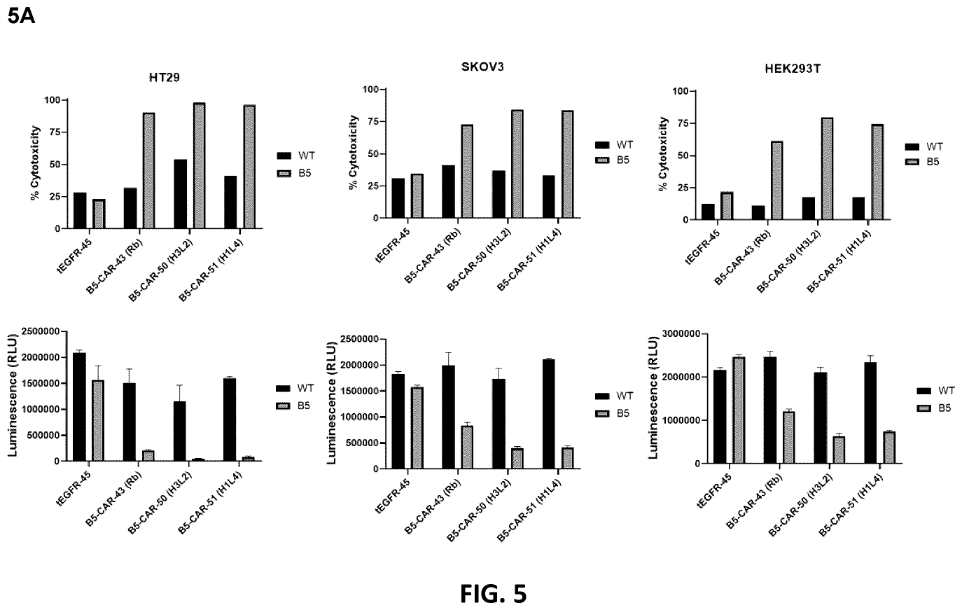

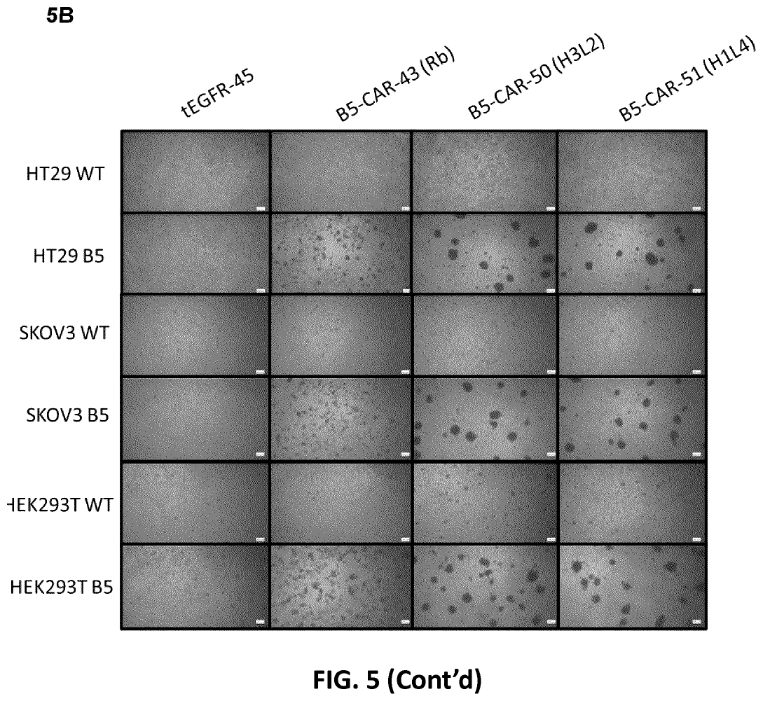

A mammalian expression transfer plasmid encoding firefly luciferase, pCCL Luc Puromycin, was used to generate lentivirus, as described in Example 9. HT-29, SKOV3, and HEK293T-WT and -B5 lines were transduced with this luciferase-encoding lentivirus and selected over 2 weeks for resistance to puromycin (1.5 μg/ml, Sigma, Cat #P8833). The expanded puromycin-resistant cell cultures were used as target populations for VV-CAR killing assays. Luciferase-expressing target HT29, SKOV3, and HEK293T lines were seeded at 2×10⁴ cells, 100 μl per well in a 96-well plate (Corning, Cat #3917) and incubated overnight to adhere. A population of expanded primary human B5-CAR-050, B5-CAR-051, parental B5-CAR-043, and vector control tEGFR T cells were plated (100 μl per well) in triplicate in a 96-well plate and diluted in a two-fold series to establish an E:T ratio spanning 20:1 to 0.625:1 relative to overall CAR expression. The T cell suspensions were then transferred to the adherent tumor cells to achieve a total volume of 200 μl. Additionally, 3 wells of target cells alone and 3 wells of media only were plated to determine maximal and minimal relative luminescence units (RLU), respectively. Cells were cultured for 24-36 h at 37° C. in 5% CO₂. Following the incubation, 22 μl 10× stock of Xenolight™ D-Luciferin (Perkin Elmer, Cat #122799) was added to each well and incubated for 10 min at RT in the dark. Plates were then scanned on a luminescence plate reader (ThermoFisher Varioskan Lux plate reader). Triplicate wells were averaged, and the percent specific cytotoxicity was determined by the following equation: percent specific cytotoxicity=100×((Max Luminescence RLU−Test Luminescence RLU)/(Max Luminescence RLU−Min Luminescence RLU)). As shown in FIG. 5A, percent specific cytotoxicity and relative luminescence for B5-CAR-050, B5-CAR-051, parental B5-CAR-043, and vector control tEGFR 1.25:1 E:T co-culture wells exhibited high percent cytotoxicity specific for B5 target cell lines and above-background cytotoxicity with the tEGFR vector control condition. Further, B5-CAR-050 and B5-CAR-051 appeared to exhibit improved overall cytotoxicity compared to that of parental B5-CAR-043. B5-expressing target cells co-cultured at 10:1 E:T with B5-CAR-050 or B5-CAR-051 T cells were also imaged for morphological signs of cell death at 24 hours, and compared to B5 CARs cultured with WT tumor cells. B5-CAR-050 and B5-CAR-051 exhibited clear cell clustering and signs of cytotoxicity when cultured with a B5 target expressing tumor cell line (FIG. 5B). Moreover, the extent of CAR clustering appeared greater than that of parental B5-CAR-043. These results show that B5-CAR-050 or B5-CAR-051 T cells efficiently and specifically kill B5-expressing tumor cells. Further, by targeting OV antigens expressed on the surface of tumor cells, target cells can be made susceptible to humanized VV-CAR mediated destruction.

### Example 11—Human T Cells Expressing B5-CAR-050 or B5-CAR-051 Demonstrate Cytotoxicity Against Tumor Cells Infected with Vaccinia Virus

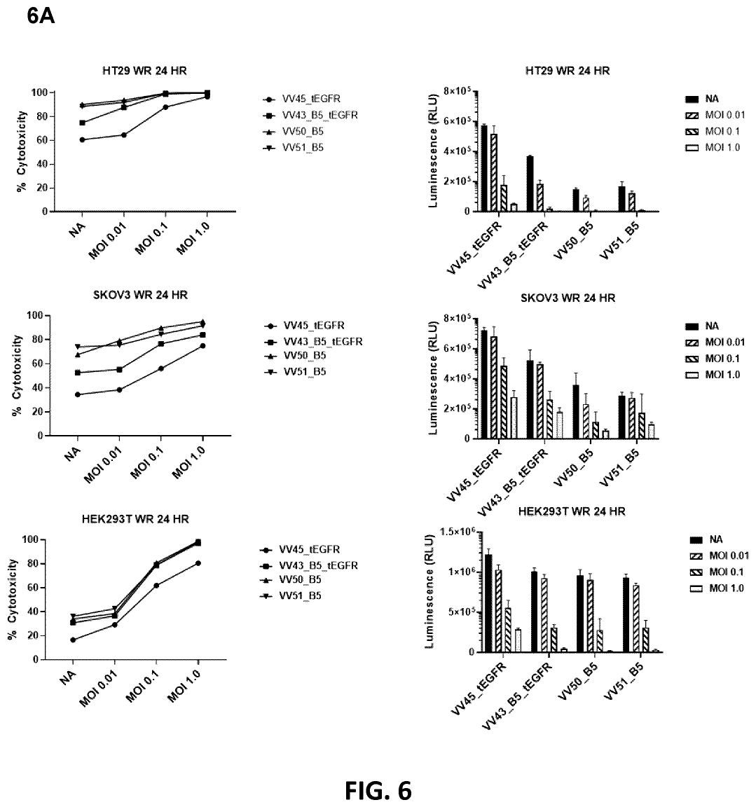

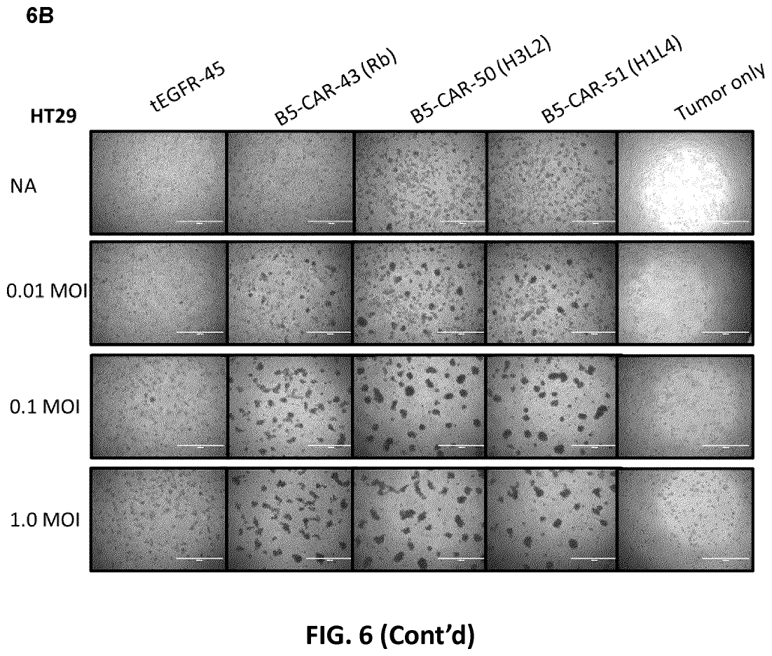

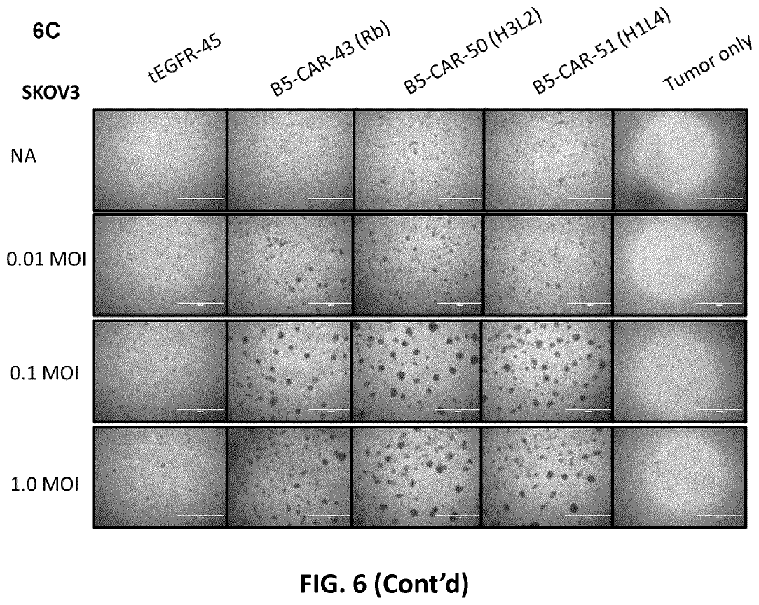

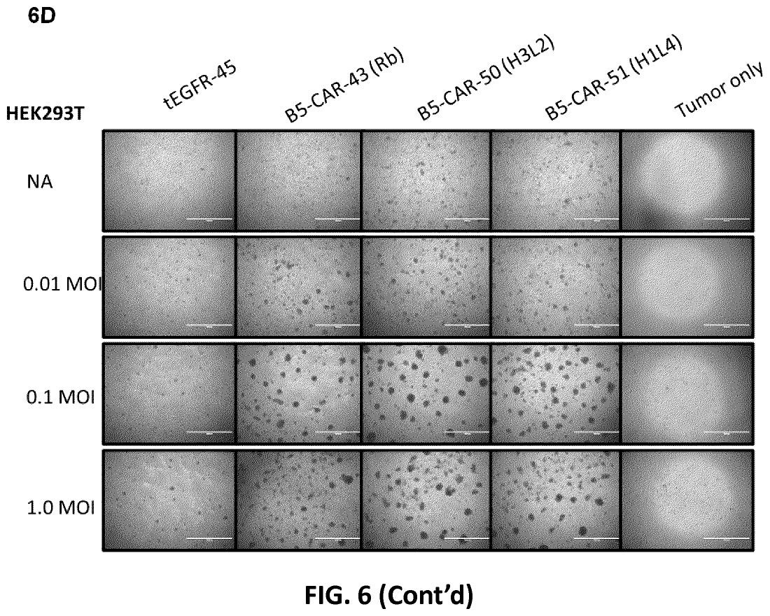

Luciferase-expressing target HT-29, SKOV3, and HEK293T WT lines were seeded at 2×10⁴ cells in 100 μl per well in a 96-well plate (Corning, Cat #3917) and incubated overnight to adhere. The following day, tumor cells were infected with Vaccinia virus (non-limiting examples of which include Western Reserve strain, Copenhagen strain, or the like) at a multiplicity of infection ranging from 0.01 to 1.0 in serum-free DMEM (Gibco, Cat #11995-040) 50 μl per well and incubated for 24 h, at 37° C. in 5% CO₂. Corresponding mock-uninfected wells are set up as a negative control. A population of expanded primary human B5-CAR-050, B5-CAR-051, parental B5-CAR-043, and vector control tEGFR T cells were plated (50 μl per well) in triplicate in vaccinia-infected plates to achieve an E:T ratio of 10:1 and total volume of 200 μl per well. Additionally, 3 wells of target cells alone (+/− infection) and 3 wells of T cells only (+/− infection) were plated to determine maximal and minimal relative luminescence units (RLU). Cells were cultured for an additional 24-48 h at 37° C. in 5% CO₂. Each well received 22 μl 10× stock of Xenolight™ 0-Luciferin (Perkin Elmer, Cat #122799) and was incubated for 10 min at RT in the dark. Plates were then scanned on a luminescence plate reader (ThermoFisher Varioskan Lux plate reader). Triplicate well RLU were averaged, and the percent specific cytotoxicity was determined as described in Example 10. As shown in FIG. 6A, percent specific cytotoxicity and relative luminescence for B5-CAR-050, B5-CAR-051, parental B5-CAR-043, and vector control tEGFR 10:1 E:T co-culture wells exhibited increased percent cytotoxicity specific for vaccinia virus-infected target cell lines and above-background cytotoxicity present with tEGFR vector control condition. Vaccinia virus infected target cells co-cultured at 10:1 E:T with B5-CAR-050 or B5-CAR-051 T cells were also imaged for morphological signs of cell death at 24 hours, and compared to B5 CARs cultured with uninfected tumor cells (FIG. 6B-6D). B5-CAR-050 and B5-CAR-051 exhibited clear cell clustering and signs of cytotoxicity when cultured with a vaccinia virus-infected tumor cell line. These findings demonstrate that B5-CAR-050 and B5-CAR-051 are able to specifically detect and kill tumor cells infected with vaccinia virus.

### Example 12—Anti-Tumor Efficacy of Humanized B5-CAR Against a Vaccinia Virus-Infected Xenograft Tumor Model

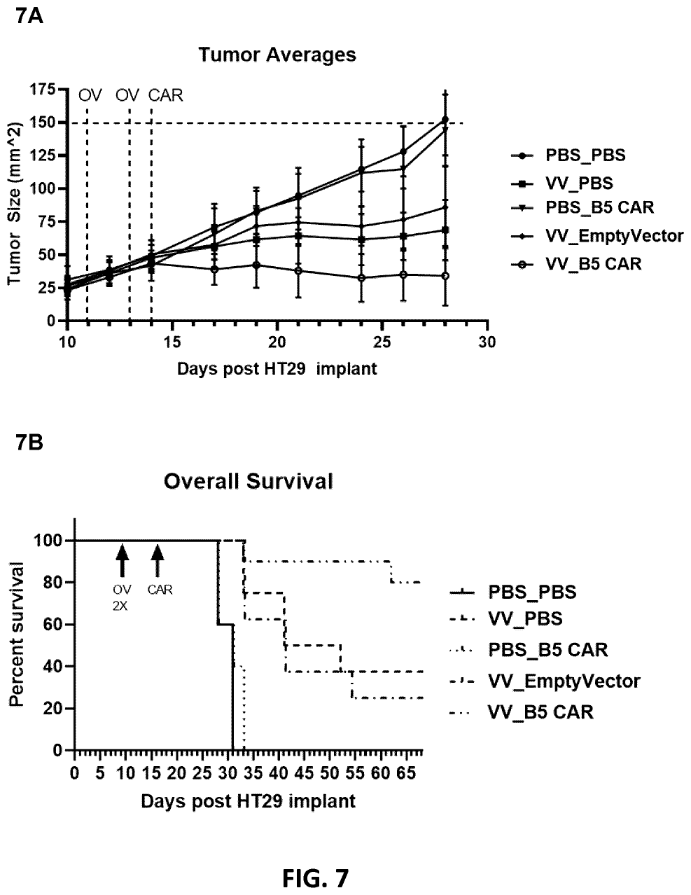

HT-29 tumor bearing mice will be treated with a combination of Vaccinia Virus and humanized B5-CAR. NSG mice will be implanted subcutaneously with 1×10⁶ cells on day 0 and monitored for tumor growth. When tumors reach a treatable size (˜30-40 mm²), mice will be injected intratumorally with 1×10⁷ PFU of vaccinia virus, receiving a total of 1-3 doses. Approximately 1-3 days following the last vaccinia virus injection, the mice will receive 5×10⁶ B5-CAR-positive T cells via tail vein injection. Tumor measurements will be recorded every 2-3 days and animals will be monitored for survival. Control groups will receive combinations of either intratumoral PBS injections and/or empty vector mock-transduced T cells delivered via tail vein injections. FIG. 7A: Expected tumor area across the various treatment conditions. FIG. 7B: Expected overall survival across the various treatment conditions.

### REFERENCES

1. Liu Y, Caffry I, Wu J, Geng S B, Jain T, Sun T, Reid F, Cao Y, Estep P, Yu Y, Vásquez M, Tessier P M, Xu Y. High-throughput screening for developability during early-stage antibody discovery using self-interaction nanoparticle spectroscopy. MAbs. 2014 March-April; 6(2):483-92. doi: 10.4161/mabs.27431. Epub 2013 Dec. 6. PMID: 24492294; PMCID: PMC3984336.
2. Shan L, Mody N, Sormani P, Rosenthal K L, Damschroder M M, Esfandiary R. Developability Assessment of Engineered Monoclonal Antibody Variants with a Complex Self-Association Behavior Using Complementary Analytical and in Silico Tools. Mol Pharm. 2018 Dec. 3; 15(12):5697-5710. doi: 10.1021/acs.molpharmaceut.8b00867. Epub 2018 Nov. 15. PMID: 30395473.
3. Avery L B, Wade J, Wang M, Tam A, King A, Piche-Nicholas N, Kavosi M S, Penn S, Cirelli D, Kurz J C, Zhang M, Cunningham O, Jones R, Fennell B J, McDonnell B, Sakorafas P, Apgar J, Finlay W J, Lin L, Bloom L, O'Hara D M. Establishing in vitro in vivo correlations to screen monoclonal antibodies for physicochemical properties related to favorable human pharmacokinetics. MAbs. 2018 February/March; 10(2):244-255. doi: 10.1080/19420862.2017.1417718. Epub 2018 Jan. 29. PMID: 29271699; PMCID: PMC5825195.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. An antibody that specifically binds to Vaccinia Virus B5 antigen (VV B5), wherein the antibody comprises:
a variable heavy chain (V_{H}) polypeptide comprising 70% or greater sequence identity to the amino acid sequence set forth in SEQ ID NO: 1, wherein the V_{H} polypeptide comprises:
one or more mutations selected from the group consisting of: Q1E, E2V, Q3T, E5L/K, G9P, G10V, K13Q, E15G/T, G16E, S17T, T19R, T21S, T23A, A41P, R44K, G45A, T74D, S75N/T, S76_T77insK, T77N/S, T78Q, V79L, T80Y/V, Q82T, T84N, R85S/N, L86M, T87R/D, A88P, A89E/V, T93V, F95Y, P112Q, and any combination thereof, wherein numbering is according to SEQ ID NO:1;
a V_{H} CDR1 comprising the amino acid sequence SSYYMC (SEQ ID NO:13),
a V_{H} CDR2 comprising the amino acid sequence CIYTSSGSAYYA(N/D)(W/S)(A/V)KG (SEQ ID NO:14), and
a V_{H} CDR3 comprising the amino acid sequence NAVGSSYYLYL (SEQ ID NO:17); and
a variable light chain (V_{L}) polypeptide comprising 70% or greater sequence identity to the amino acid sequence set forth in SEQ ID NO: 7, wherein the V_{L} polypeptide comprises:
one or more mutations selected from the group consisting of: A1D/E, Q2I, V3Q, L4M, T7S, S9A, P10T/S, V11L, A13L, A14S, V15P, G17D/E, T18R, V19A, I21L, S22T, Q44K, P45A/V, N47K/R, V60I, S62A, K65S, Q72E/D, D79S, E81Q, C82P, D83E, A85F/V, T87V, G103Q, E106K, V107L, V108E, V109I, and any combination thereof, wherein numbering is according to SEQ ID NO: 7;
a V_{L} CDR1 comprising the amino acid sequence QASQSVAGNNYLS (SEQ ID NO:18),
a V_{L} CDR2 comprising the amino acid sequence SVSTLAS (SEQ ID NO:19), and
a V_{L} CDR3 comprising the amino acid sequence QGYYNDGIWA (SEQ ID NO:20).

2. The antibody of claim 1, wherein the V_{H} polypeptide comprises one, any combination of, or each of the mutations E2V, E5L/K, E15G/T, R44K, R85S/N, T87R/D, A89E/V, and P112Q.

3. The antibody of claim 2, wherein the V_{H} polypeptide comprises each of the mutations E2V, E5L/K, E15G/T, R44K, R85S/N, T87R/D, A89E/V, P112Q.

4. The antibody of claim 2 or claim 3, wherein the E5L/K mutation is E5L.

5. The antibody of any one of claims 2 to 4, wherein the E15G/T mutation is E15G.

6. The antibody of any one of claims 2 to 5, wherein the R85S/N mutation is R85S.

7. The antibody of any one of claims 2 to 6, wherein the T87R/D mutation is T87R.

8. The antibody of any one of claims 2 to 7, wherein the A89E/V mutation is A89E.

9. The antibody of any one of claims 2 to 8, wherein the V_{H} polypeptide comprises one, any combination of, or each of the mutations Q1E, K13Q, T19R, T21S, T23A, T84N, T93V, and F95Y.

10. The antibody of any one of claims 2 to 9, wherein the V_{H} polypeptide comprises a V_{H} CDR2 comprising the amino acid sequence CIYTSSGSAYYADSVKG (SEQ ID NO:16).

11. The antibody of any one of claims 2 to 9, wherein the V_{H} polypeptide comprises one, any combination of, or each of the mutations T74D, S75N/T, S76_T77insK, T77N/S, V79L, and T80Y/V.

12. The antibody of claim 11, wherein the T77N/S mutation is T77N.

13. The antibody of claim 11 or claim 12, wherein the T80Y/V mutation is T80Y.

14. The antibody of any one of claims 11 to 13, wherein the V_{H} polypeptide comprises a V_{H} CDR2 comprising the amino acid sequence CIYTSSGSAYYADSVKG (SEQ ID NO:16).

15. The antibody of claim 2 or claim 3, wherein the V_{H} polypeptide comprises one, any combination of, or each of the mutations Q3T, E5L/K, G9P, G10V, E15G/T, G16E, S17T, A41P, G45A, T74D, S75N/T, S76_T77insK, T77N/S, T78Q, T80Y/V, Q82T, R85S/N, L86M, T87R/D, A88P, and A89E/V.

16. The antibody of claim 15, wherein the E5L/K mutation is E5K.

17. The antibody of claim 15 or claim 16, wherein the E15G/T mutation is E15T.

18. The antibody of any one of claims 15 to 17, wherein the S75N/T mutation is S75T.

19. The antibody of any one of claims 15 to 18, wherein the T77N/S mutation is T77S.

20. The antibody of any one of claims 15 to 19, wherein the T80Y/V mutation is T80V.

21. The antibody of any one of claims 15 to 20, wherein the R85S/N mutation is R85N.

22. The antibody of any one of claims 15 to 21, wherein the T87R/D mutation is T87D.

23. The antibody of any one of claims 15 to 22, wherein the A89E/V mutation is A89V.

24. The antibody of any one of claims 1 to 3, wherein the V_{H} polypeptide comprises 80% or greater, 85% or greater, 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in one of SEQ ID Nos: 2-6.

25. The antibody of any one of claims 2 to 9, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 3.

26. The antibody of claim 10, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 5.

27. The antibody of any one of claims 11 to 13, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 2.

28. The antibody of claim 14, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 4.

29. The antibody of any one of claims 15 to 23, wherein the V_{H} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 6.

30. The antibody of any one of claims 1 to 29, wherein the V_{L} polypeptide comprises one, any combination of, or each of the mutations T7S, P10T/S, V11L, A14S, G17D/E, T18R, P45A/V, N47K/R, K65S, Q72E/D, D79S, C82P, A85F/V, G103Q, E106K, V108E, and V109I.

31. The antibody of claim 30, wherein the V_{L} polypeptide comprises each of the mutations T7S, P10T/S, V11L, A14S, G17D/E, T18R, P45A/V, N47K/R, K65S, Q72E/D, D79S, C82P, A85F/V, G103Q, E106K, V108E, and V109I.

32. The antibody of claim 30 or claim 31, wherein the V_{L} polypeptide comprises one, any combination of, or each of the mutations G17D, S22T, Q44K, N47K, and E81Q.

33. The antibody of any one of claims 30 to 32, wherein the P10T/S mutation is P10T.

34. The antibody of any one of claims 30 to 33, wherein the P45A/V mutation is P45A.

35. The antibody of any one of claims 30 to 34, wherein the Q72E/D mutation is Q72E.

36. The antibody of any one of claims 30 to 35, wherein the A85F/V mutation is A85F.

37. The antibody of any one of claims 33 to 36, wherein the V_{L} polypeptide comprises one, any combination of, or each of the mutations A1D, Q2I, V3Q, and L4M.

38. The antibody of any one of claims 30 to 32, wherein the V_{L} polypeptide comprises the mutation D83E.

39. The antibody of any one of claims 30 to 32 or 38, wherein the P10T/S mutation is P10S.

40. The antibody of any one of claims 30 to 32, 38, or 39, wherein the P45A/V mutation is P45V.

41. The antibody of any one of claims 30 to 32 or 38 to 40, wherein the Q72E/D mutation is Q72D.

42. The antibody of any one of claims 30 to 32 or 38 to 41, wherein the A85F/V mutation is A85V.

43. The antibody of any one of claims 38 to 42, wherein the V_{L} polypeptide comprises one, any combination of, or each of the mutations A1D, Q2I, V3Q, and L4M.

44. The antibody of claim 30 or claim 31, wherein the V_{L} polypeptide comprises one, any combination of, or each of the mutations A1E, S9A, P10T, A13L, V15P, G17E, V19A, I21L, P45A, N47R, V60I, S62A, Q72D, D83E, A85F, T87V, and V107L.

45. The antibody of claim 44, wherein the V_{L} polypeptide comprises each of the mutations A1E, S9A, P10T, A13L, V15P, G17E, V19A, I21L, P45A, N47R, V60I, S62A, Q72D, D83E, A85F, T87V, and V107L.

46. The antibody of claim 1, 30 or 31, wherein the V_{L} polypeptide comprises 80% or greater, 85% or greater, 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in one of SEQ ID Nos: 8-12.

47. The antibody of any one of claims 33 to 36, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 9.

48. The antibody of claim 37, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 8.

49. The antibody of any one of claims 38 to 42, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 11.

50. The antibody of claim 43, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 10.

51. The antibody of claim 44 or claim 45, wherein the V_{L} polypeptide comprises 90% or greater, 91% or greater, 92% or greater, 93% or greater, 94% or greater, 95% or greater, 96% or greater, 97% or greater, 98% or greater, 99% or greater, or 100% sequence identity to the amino acid sequence set forth in SEQ ID No: 12.

52. An antibody that specifically binds to Vaccinia Virus B5 antigen (VV E5), wherein the antibody comprises:
a variable heavy chain (V_{H}) polypeptide comprising:
a V_{H} CDR1 comprising the amino acid sequence SSYYMC (SEQ ID NO:13),
a V_{H} CDR2 comprising the amino acid sequence CIYTSSGSAYYADSVKG (SEQ ID NO:16), and
a V_{H} CDR3 comprising the amino acid sequence NAVGSSYYLYL (SEQ ID NO:17); and
a variable light chain (V_{L}) polypeptide comprising:
a V_{L} CDR1 comprising the amino acid sequence QASQSVAGNNYLS (SEQ ID NO:18),
a V_{L} CDR2 comprising the amino acid sequence SVSTLAS (SEQ ID NO:19), and
a V_{L} CDR3 comprising the amino acid sequence QGYYNDGIWA (SEQ ID NO:20).

53. The antibody of claim 52, wherein the antibody is a humanized antibody.

54. The antibody of any one of claims 1 to 53, wherein the antibody is an IgG.

55. The antibody of claim 54, wherein the antibody comprises a human Fc domain.

56. The antibody of any one of claims 1 to 53, wherein the antibody is selected from the group consisting of: a Fab, a F(ab′)₂, and a F(ab′).

57. The antibody of any one of claims 1 to 53, wherein the antibody is a single chain antibody.

58. The antibody of claim 57, wherein the single chain antibody is an scFv.

59. The antibody of any one of claims 1 to 58, wherein the antibody is a bispecific antibody comprising a first antigen-binding domain comprising a V_{H} polypeptide-V_{L} polypeptide pair as defined in any one of claims 1 to 53.

60. The antibody of claim 59, wherein the bispecific antibody comprises a second antigen-binding domain that specifically binds an antigen other than a Vaccinia Virus B5 antigen.

61. The antibody of claim 60, wherein the antigen other than a Vaccinia Virus B5 antigen is an immune cell surface antigen.

62. The antibody of claim 61, wherein the immune cell surface antigen is an immune effector cell surface antigen.

63. The antibody of claim 62, wherein the immune cell surface antigen is a T cell surface antigen.

64. The antibody of claim 63, wherein the antigen is a T cell stimulatory molecule.

65. The antibody of claim 64, wherein the T cell stimulatory molecule is CD3 or CD28.

66. The antibody of claim 62, wherein the immune cell surface antigen is a natural killer (NK) cell surface antigen.

67. The antibody of claim 62, wherein the immune cell surface antigen is a macrophage cell surface antigen.

68. A fusion protein, comprising:
a chain of an antibody of any one of claims 1 to 53 fused to a heterologous sequence of amino acids.

69. The fusion protein of claim 68, wherein the heterologous sequence of amino acids is fused to the C-terminus of the chain of the antibody.

70. The fusion protein of claim 68 or claim 69, wherein the antibody is the single chain antibody of claim 57 or 58.

71. The fusion protein of claim 70, wherein the fusion protein is a chimeric antigen receptor (CAR) comprising:
the single chain antibody;
a transmembrane domain; and
an intracellular signaling domain.

72. A conjugate, comprising:
an antibody of any one of claims 1 to 67 or a fusion protein of any one of claims 68 to 71; and
an agent conjugated to the antibody or fusion protein.

73. The conjugate of claim 72, wherein the agent is selected from the group consisting of: a chemotherapeutic agent, a toxin, a radiation sensitizing agent, a radioactive isotope, a detectable label, and a half-life extending moiety.

74. The conjugate of claim 73, wherein the radioactive isotope is a therapeutic radioactive isotope.

75. The conjugate of claim 73, wherein the detectable label is a radiolabel.

76. The conjugate of claim 75, wherein the radiolabel is Zirconium-89 (⁸⁹Zr).

77. The conjugate of any one of claims 72 to 76, wherein the agent is conjugated to the antibody or fusion protein via a non-cleavable linker.

78. The conjugate of any one of claims 72 to 76, wherein the agent is conjugated to the antibody or fusion protein via a cleavable linker.

79. The conjugate of claim 78, wherein the cleavable linker is an enzyme-cleavable linker.

80. The conjugate of claim 79, wherein the linker is cleavable by a lysosomal protease.

81. The conjugate of claim 80, wherein the linker is cleavable by cathepsin or plasmin.

82. A nucleic acid encoding a variable heavy chain (V_{H}) polypeptide, a variable light chain (V_{L}) polypeptide, or both, of an antibody of any one of claims 1 to 67.

83. A nucleic acid encoding the fusion protein of any one of claims 68 to 71.

84. An expression vector comprising the nucleic acid of claim 82 or claim 83.

85. A cell comprising:
the nucleic acid of claim 82 or claim 83; or
the expression vector of claim 84.

86. The cell of claim 85, wherein the cell comprises the nucleic acid of claim 83 or an expression vector comprising the nucleic acid of claim 83.

87. The cell of claim 86, wherein the nucleic acid encodes the CAR of claim 71 and the cell expresses the CAR on its surface.

88. The cell of claim 87, wherein the cell is an immune cell.

89. The cell of claim 88, wherein the cell is an immune effector cell.

90. The cell of claim 89, wherein the cell is a T cell.

91. The cell of claim 89, wherein the cell is an NK cell.

92. The cell of claim 89, wherein the cell is a macrophage.

93. A cell comprising:
a first nucleic acid encoding the variable heavy chain (V_{H}) polypeptide of an antibody of any one of claims 1 to 53; and
a second nucleic acid encoding the variable light chain (V_{L}) polypeptide of the antibody.

94. The cell of claim 93, comprising:
a first expression vector comprising the first nucleic acid; and
a second expression vector comprising the second nucleic acid.

95. A pharmaceutical composition comprising the cell of any one of claims 85 to 94.

96. The pharmaceutical composition of claim 95, comprising the cell of any one of claims 87 to 92.

97. The pharmaceutical composition of claim 95 or claim 96, further comprising a pharmaceutically acceptable carrier.

98. A method of producing the antibody of any one of claims 1 to 53, comprising culturing the cell of any one of claims 85 to 94 under conditions suitable for the cell to express the antibody, wherein the antibody is produced.

99. A pharmaceutical composition, comprising:
the antibody of any one of claims 1 to 67; and
a pharmaceutically acceptable carrier.

100. A pharmaceutical composition, comprising:
the fusion protein of any one of claims 68 to 71; and
a pharmaceutically acceptable carrier.

101. A pharmaceutical composition, comprising:
the conjugate of any one of claims 72 to 81; and
a pharmaceutically acceptable carrier.

102. A kit, comprising:
the pharmaceutical composition of any one of claims 95 to 97 or 99 to 101; and
instructions for administering the pharmaceutical composition to an individual in need thereof.

103. The kit of claim 102, wherein the pharmaceutical composition is present in one or more unit dosages.

104. The kit of claim 102, wherein the pharmaceutical composition is present in two or more unit dosages.

105. A method, comprising:
administering the pharmaceutical composition of any one of claims 95 to 97 or 99 to 101 to an individual having cancer, wherein the individual comprises cancer cells infected with Vaccinia Virus (VV), and wherein the antibody, fusion protein or conjugate is targeted to the infected cancer cells by VV B5 antigens expressed on the surface of the infected cancer cells.

106. The method according to claim 105, further comprising, prior to administering the pharmaceutical composition to the individual, infecting the cancer cells by administering VV to the individual.

107. The method according to claim 105 or 106, wherein the method is a method of treating the cancer of the individual.

108. The method according to any one of claims 105 to 107, wherein the pharmaceutical composition of claim 101 is administered to the individual, wherein the conjugate comprises the antibody conjugated to a detectable label or radioactive isotope which is an in vivo imaging agent, and wherein the method comprises imaging the infected cancer cells in the individual using the in vivo imaging agent.
